(19) United States
(12) Patent Application Publication
(10) Pub. No.: US 2023/0293526 A1
(43) Pub. Date: Sep. 21, 2023

(54) METHOD FOR TREATING CANCER WITH A REVERSE TRANSCRIPTASE INHIBITOR

(71) Applicant: PrimeFour Therapeutics, Inc., Menlo Park, CA (US)

(72) Inventor: Eckard WEBER, San Diego, CA (US)

(21) Appl. No.: 18/188,765

(22) Filed: Mar. 23, 2023

Related U.S. Application Data
(63) Continuation-in-part of application No. PCT/US2021/051718, filed on Sep. 23, 2021.
(60) Provisional application No. 63/082,207, filed on Sep. 23, 2020.
(60) Provisional application No. 63/165,270, filed on Mar. 24, 2021.
(60) Provisional application No. 63/178,379, filed on Apr. 22, 2021.
(60) Provisional application No. 63/184,051, filed on May 4, 2021.

(51) Int. Cl. A61K 31/513; A61K 31/52; A61P 35/00; A61K 45/06

(52) U.S. Cl. CPC A61K 31/513; A61K 31/52; A61P 35/00; A61K 45/06

(57) ABSTRACT

Disclosed is a method for treating cancer in patient in need thereof comprising administering a therapeutically effective amount of a nucleoside, e.g., a reverse transcriptase inhibitor (RTI), to the patient according to a continuous or an intermittent dosing schedule. RTIs include, but are not limited to, lamivudine (3TC), stavudine (d4T), emtricitabine (FTC), abacavir (ABC), tenofovir alafenamide, zidovudine (AZT), zalcitabine (ddC), didanosine (ddI), tenofovir disoproxil, adefovir dipivoxil, entecavir (ETV), telbivudine, and islatravir.

### CROSS-REFERENCE TO RELATED APPLICATIONS

This application is a continuation-in-part of PCT/US2021/051718, filed Sep. 23, 2021, that claims the benefit of U.S. Provisional Application No. 63/082,207, filed Sep. 23, 2020; U.S. Provisional Application No. 63/165,270, filed Mar. 24, 2021; U.S. Provisional Application No. 63/178,379, filed Apr. 22, 2021; and U.S. Provisional Application No. 63/184,051, filed May 4, 2021, which are incorporated herein by reference in their entirety.

### BACKGROUND

### Field

The present disclosure is in the field of medicinal chemistry. In particular, the disclosure provides a method for treating cancer by administering a nucleoside, e.g., a reverse transcriptase inhibitor (RTI), to a patient in need thereof according to a continuous or an intermittent dosing schedule. Exemplary RTIs include lamivudine (3TC), stavudine (d4T), emtricitabine (FTC), abacavir (ABC), tenofovir alafenamide, zidovudine (AZT), zalcitabine (ddC), didanosine (ddI), tenofovir disoproxil, adefovir dipivoxil, entecavir (ETV), islatravir, and telbivudine. In one embodiment, the cancer is breast, colon, lung, pancreatic ductal, prostate, ovarian, or head and neck cancer.

### Background

Long INterspersed Element-1 (LINE-1 or L1) retrotransposons form the only autonomously active family of transposable elements in humans. They are expressed and mobile in the germline, in embryonic stem cells, and in the early embryo, but are silenced in most somatic tissues. LINE-1 plays an important role in individual genome variations through insertional mutagenesis and sequence transduction, which occasionally lead to genetic diseases and disorders. In addition, LINE-1 is reactivated in certain cancers thus contributing to tumor genome dynamics. The LINE-1 element codes for two proteins, ORF1 p and ORF2p, which are essential for its mobility. ORF1p is an RNA-binding protein with nucleic acid chaperone activity. ORF2p possesses endonuclease and reverse transcriptase activities. These proteins and the LINE-1 RNA assemble into a ribonucleoprotein particle (LINE-1 RNP)—the core of the retrotransposition machinery. The LINE-1 RNP mediates the synthesis of new LINE-1 copies upon cleavage of the target DNA and reverse transcription of the LINE-1 RNA at the target site. The LINE-1 element takes benefit of cellular host factors to complete its life cycle, however several cellular pathways also limit the cellular accumulation of LINE-1 RNPs and their deleterious activities. See, e.g., Pizarro and Cristofari (2016) Front. Cell Dev. Biol. 4:14. doi: 10.3389/fcell.2016.00014.

LINE-1 retrotransposition is a hallmark of cancer. See, e.g., Rodic, N., Frontiers InBioscience (Landmark Ed.) 23:1680-1686 (2018); Xiao-Jie et al., Genet Med 18:431-439 (2016); and Zhang et al., Front Cell Dev Biol. 8:657 (2020) https://doi.org/10.3389/fcell.2020.00657. Increased expression of LINE-1 promotes pathogenesis by damaging the host DNA via mutation insertions and altering target gene expression and chromosomal rearrangements. To this end, LINE-1 methylation studies have been conducted in common lethal cancers, lung cancer, colon and rectal cancers, breast cancer, prostate cancer, liver cancer, ovarian cancer, and esophageal cancer. See, e.g., Ardeljan, et al, Clinical Chemistry 63:816-822 (2017). In non-small cell lung cancer, LINE-1 promoter hypomethylation is common and is associated with genomic instability and poor prognosis. In colon cancer, LINE-1 hypomethylation appears to be an early event also associated with poor outcomes. It is more pronounced in colon cancer liver metastases compared to matched primary tumors. In breast cancer, LINE-1 hypomethylation has been reported in preneoplastic phases of epithelial atypia with persistently low LINE-1 promoter methylation seen in in situ and invasive lesions. It has also been associated with decreased overall survival and drug resistance in younger patients. In prostate cancers, LINE-1 hypomethylation is also reported, particularly in association with chromosome 8 abnormalities; it appears to be more pronounced in metastatic lesions than in primary tumors. In hepatocellular carcinoma, several groups have associated LINE-1 hypomethylation with poor clinical outcomes, including disease recurrence after resection. In epithelial ovarian cancers, LINE-1 hypomethylation is correlated with more aggressive histology, poorer progression-free intervals, and poorer survival. Lastly, in esophageal squamous cell carcinomas, LINE-1 hypomethylation is also recognized and associated with poorer survival.

There is a need in the art for LINE-1 inhibitors for use in treating cancer.

### BRIEF SUMMARY

In some embodiments, provided is a method for treating cancer, e.g., breast, colon, lung, pancreatic ductal, prostate, ovarian, or head and neck cancer, in patient in need thereof comprising administering a therapeutically effective amount of a RTI to the patient, wherein the RTI is administered according to a continuous or an intermittent dosing schedule.

In some embodiments, provided is a method for treating cancer, e.g., breast, colon, lung, pancreatic ductal, prostate, ovarian, or head and neck cancer, in patient in need thereof comprising administering a therapeutically effective amount of lamivudine (3TC), stavudine (d4T), emtricitabine (FTC), abacavir (ABC), tenofovir alafenamide, zidovudine (AZT), zalcitabine (ddC), didanosine (ddI), tenofovir disoproxil, adefovir dipivoxil, entecavir (ETV), or telbivudine to the patient in need thereof according to a continuous or an intermittent dosing schedule.

In some embodiments, provided is a method for treating cancer, e.g., breast, colon, lung, pancreatic ductal, prostate, ovarian, or head and neck cancer, in patient in need thereof comprising administering a therapeutically effective amount of a nucleoside, e.g., Cpd. No. 7, Cpd. No. 9, Cpd. No. 13, Cpd. No. 15, Cpd. No. 20, Cpd. No. 21, Cpd. No. 25, Cpd. No. 25, Cpd. No. 30, Cpd. No. 31, Cpd. No. 32, islatravir, or elvucitabine, to the patient.

In some embodiments, provided is a method for the treatment of breast cancer. In some embodiments, provided is a method for the treatment of colon cancer. In some embodiments, provided is a method the treatment of lung cancer. In some embodiments, provided is a method for the treatment of pancreatic ductal cancer. In some embodiments, provided is a method for the treatment of prostate cancer. In some embodiments, provided is a method for the treatment high-risk localized prostate cancer. In some embodiments, provided is a method for the treatment of prostate cancer that is not metastatic. In some embodiments, provided is a method for the treatment of ovarian cancer. In some embodiments, provided is a method for the treatment of head and neck cancer.

In some embodiments, provided is a method further comprising administering a therapeutically effective amount of at least one second therapeutic agent useful for treating the cancer.

In some embodiments, provided is a method for the treatment of breast cancer, wherein the at least one second therapeutic agent is Soltamox® (tamoxifen), Arimidex® (anastrozole), Femara® (letrozole), Aromasin® (exemestane), Herceptin® (trastuzumab), Abraxane® (paclitaxel), Cytoxan® (cyclophosphamide), Taxol® (paclitaxel), Afinitor® (everolimus), Taxotere® (docetaxel), Xeloda® (capecitabine), Trexall® (methotrexate), Faslodex (fulvestrant), Adriamycin® (doxorubicin), Perjeta® (pertuzumab), Gemzar (gemcitabine), Tykerb® (lapatinib), Adrucil® (fluorouracil), Ibrance® (palbociclib), Verzenio® (abemaciclib), Fareston® (toremifene), Halaven® (eribulin), Menest, Kadcyla® (ado-trastuzumab emtransine), Androxy® (fluoxymesterone), Avastin® (bevacizumab), esterified estrogens, Herzuma® (trastuzumab), Ixempra® (ixabepilone), Kanjinti® (trastuzumab), Kisqali® (ribociclib), Ogivri® (trastuzumab), Ontruzant® (trastuzumab), Tepadina® (thiotepa), Trazimera® (trastuzumab), Velban® (vinblastine), Piqray® (alpelisib), Tecentriq® (atezolizumab), Enhertu® (fam-trastuzumab deruxtecan), Herceptin, Hylecta™ (hyaluronidase/trastuzumab), Infugem® (gemcitabine), Kisqali® Femara® Co-Pack (ribociclib and letrozole), Talzenna® (talazoparib), Trodelvy® (sacituzumab) or Tukysa™ (tukatinib).

In some embodiments, provided is a method for the treatment of colon cancer, wherein the at least one second therapeutic agent is Xeloda® (capecitabine), Eloxatin® (oxaliplatin), fluorouracil, Avastin® (bevacizumab), leucovorin, Camptosar® (irinotecan), Stivarga® (regorafenib), Erbitux® (cetuximab), Vectibix® (panitumumab), Lonsurf® (tipiracil/trifluridine), Zaltrap® (ziv-aflibercept), Betaseron® (interferon beta-1b), Fusilev® (levoleucovorin), Wellcocorin® (methotrexate), Keytruda® (pembrolizumab), Mvasi® (bevacizumab-awwb), Cyramza® (ramucirumab), Yervoy® (ipilmumab), Opdivo® (nivolumab), Braftovi® (encorafenib), Khapzory® (levoleucovorin) or Zirabev® (bevacizumab-bvzr).

In some embodiments, provided is a method for the treatment of lung cancer, wherein the at least one second therapeutic agent is Etopophos® (etoposide), Hycamtin® (topotecan), VePesid® (etoposide), Toposar® (etoposide), Opdivo® (nivolumab), Keytruda® (pembrolizumab), Tecentriq® (atezolizumab), Imfinizi® (durvalumab), methotrexate, cyclophosphamide, Carboplatin, Cisplatin, docetaxel, Gemcitabine, Irinotecan, Paclitaxel, Pemetrexed, Vinblastine, or Vinorelbine.

In some embodiments, provided is a method for the treatment of pancreatic ductal cancer, wherein the at least one second therapeutic agent is Gemzar® (Gemcitabine), fluorouracil, Afinitor® (everolimus), Tarceva® (erlotinib), Abraxane® (paclitaxel), capecitabine, Sutent® (sunitinib), pancreatin, methotrexate, Zanosar® (streptozocin), Mutamycin® (mitomycin), Onivyde® (irinotecan), bevacizumab, cetuximab, Infugem® (gemcitabine) or Lynparza® (olaparib).

In some embodiments, provided is a method for the treatment of head and neck cancer, wherein the at least one second therapeutic agent is Erbituz® (cetuximab), Taxotere® (docetaxel), Trexall® (methotrexate), Keytruda® (pembrolizumab) or Opdivo® (nivolumab).

In some embodiments, provided is a method for the treatment of prostate cancer, wherein the at least one second therapeutic agent is Suprefact® (buserelin), Firmagon® (degarelix), Zoladex® (goserelin), Vantas® (histrelin), Eligard® (leuprolide), Orgovyx® (relugolix), Trelstar® (triptorelin), Casodex® (bicalutamide), Eulexin® (flutamide), Nilandron® (nilutamide), Zytiga® (biraterone acetate), Erleada® (apalutamide), or Xtandi® (enzalutamide).

In some embodiments, the at least one second therapeutic agent is a STING agonist.

In some embodiments, provided is a kit for carrying out the therapeutic methods and uses of the disclosure, the kit comprising (i) a RTI; and (ii) and instructions for administering the RTI to a patient having cancer according to a continuous or an intermittent dosing schedule.

### BRIEF DESCRIPTION OF DRAWINGS

FIG. 1 is a dose response curve showing the cell proliferation activity of Compound 13 in MV4-11 cells after incubation for 72 h.

FIG. 2 is a dose response curve showing the cell proliferation activity of Compound 9 in MINO cells after incubation for 72 h.

FIG. 3 is a dose response curve showing the cell proliferation activity of Compound 9 in MINO cells after incubation for 120 h.

FIG. 4 is a dose response curve showing the cell proliferation activity of Compound 9 in MINO cells after incubation for 168 h.

### DETAILED DESCRIPTION

In one embodiment, provided is a method for treating cancer in patient in need thereof comprising administering a therapeutically effective amount of a reverse transcriptase inhibitor (RTI), e.g., islatravir, elvucitabine, lamivudine (3TC), zidovudine (AZT), tenofovir, tenofovir disoproxil, tenofovir alafenamide, stavudine (d4T), zalcitabine (ddC), didanosine (ddI), emtricitabine (FTC), entecavir (ETV) or abacavir (ABC), to the patient, wherein the RTI is administered according to a continuous or an intermittent dosing schedule.

In another embodiment the RTI is administered to the patient on a continuous dosing schedule, e.g., the RTI is administered to the patient every day.

In another embodiment the RTI is administered to the patient on an intermittent dosing schedule, e.g., the RTI is administered to the patient every other day.

In another embodiment, the RTI is administered to the patient as an adjuvant therapy to treat cancer.

In another embodiment, the RTI is administered to the patient as a neoadjuvant therapy to treat cancer.

In one embodiment, the RTI is a nucleoside, e.g., a nucleoside reverse transcriptase inhibitor (NRTI). Non-limiting exemplary NRTIs include abacavir (ZIAGEN™), abacavir/lamivudine (Epzicom), abacavir/lamivudine/zidovudine (TRIZIVIR™), adefovir, alovudine, amdoxovir, apricitabine, ATRIPLA®, BARACLUDE®, BIKTARVY®, COVIRACIL™, DAPD/DXG, D-D4FC, dexelvucitabine, didanosine (VIDEX™), didanosine extended-release (Videx EC), dOTC, emtricitabine (EMTRIVA™), emtricitabine/tenofovir alafenamide (DESCOVY®), emtricitabine/tenofovir disoproxil fumarate (TRUVADA®), fosalvudine, lamivudine/zidovudine (COMBIVIR™), EVIPLERA™, GENVOYA®, HMD™, KIVEXA™, lamivudine (EPIVIR™), LODENOSINE™, ODEFSEY®, PREVEON®, racivir, stampidine, stavudine (ZERIT™), STRIBILD®, TENOFOVIR™, tenofovir disoproxil fumarate (VIREAD™), TRIUMEQ®, Trizivir, VEMLIDY®, and zidovudine (RETROVIR™).

In another embodiment, the RTI is a non-nucleoside reverse transcriptase inhibitor (NNRTI). Non-limiting exemplary NNRTIs include delavirdine, efavirenz, etravirine, nevirapine, and rilvipirine.

In another embodiment, the RTI is a LINE-1 inhibitor.

In another embodiment, the RTI is islatravir, elvucitabine, lamivudine (3TC), zidovudine (AZT), tenofovir, tenofovir disoproxil, tenofovir alafenamide, stavudine (d4T), zalcitabine (ddC), didanosine (ddI), emtricitabine (FTC), entecavir (ETV), 2′,3′-dideoxyguanosine (ddG), 2′,3′-dideoxyadenosine (ddA), 2′-fluoro-2′,3′-dideoxyarabinosyladenine (F-ddA), efavirenz (EFV), nevirapine (NVP), or abacavir (ABC).

In another embodiment, the RTI is lamivudine (3TC), stavudine (d4T), emtricitabine (FTC), abacavir (ABC), tenofovir alafenamide, zidovudine (AZT), zalcitabine (ddC), didanosine (ddI), tenofovir disoproxil, adefovir dipivoxil, entecavir (ETV), or telbivudine.

In another embodiment, the RTI is lamivudine (3TC). In another embodiment, the RTI is stavudine (d4T). In another embodiment, the RTI is emtricitabine (FTC). In another embodiment, the RTI is abacavir (ABC). In another embodiment, the RTI is tenofovir alafenamide. In another embodiment, the RTI is zidovudine (AZT). In another embodiment, the RTI is zalcitabine (ddC). In another embodiment, the RTI is didanosine (ddI). In another embodiment, the RTI is tenofovir disoproxil. In another embodiment, the RTI is adefovir dipivoxil. In another embodiment, the RTI is entecavir. In another embodiment, the RTI is telbivudine.

In another embodiment, the RTI is a compound of Formula I:

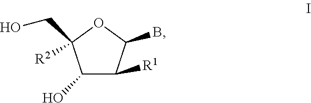

or a pharmaceutically acceptable salt or solvate thereof, or a tautomer thereof, wherein:

B is selected from the group consisting of:

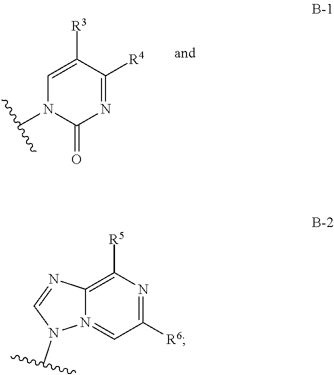

R¹ is selected from the group consisting of hydrogen and —OH;

R² is selected from the group consisting of methyl, ethynyl, and —CN;

R³ is selected from the group consisting of hydrogen, fluoro, chloro, bromo, iodo and methyl;

R⁴ is selected from the group consisting of —NH₂ and —OH;

R⁵ is selected from the group consisting of —NH₂ and —OH; and

R⁶ is selected from the group consisting of hydrogen, fluoro, chloro, and —NH₂

In another embodiment, the RTI is a compound is a compound of Formula II:

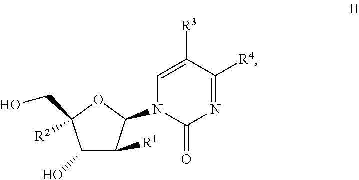

or a pharmaceutically acceptable salt or solvate thereof, or a tautomer thereof, wherein R₁, R², R³, and R⁴ are as defined in connection with Formula I.

In another embodiment, the RTI is a compound is a compound of Formula II, or a pharmaceutically acceptable salt or solvate thereof, or a tautomer thereof, wherein R³ is hydrogen. In another embodiment, the RTI is a compound is a compound of Formula II, or a pharmaceutically acceptable salt or solvate thereof, or a tautomer thereof, wherein R³ is selected from the group consisting of fluoro and chloro. In another embodiment, the RTI is a compound is a compound of Formula II, or a pharmaceutically acceptable salt or solvate thereof, or a tautomer thereof, wherein R³ is methyl.

In another embodiment, the RTI is a compound is a compound of Formula II, or a pharmaceutically acceptable salt or solvate thereof, or a tautomer thereof, wherein R⁴ is —NH₂. In another embodiment, the RTI is a compound is a compound of Formula II, or a pharmaceutically acceptable salt or solvate thereof, or a tautomer thereof, wherein R⁴ is —OH.

In another embodiment, the RTI is a compound is a compound of Formula III:

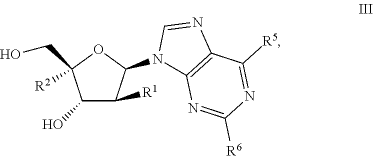

or a pharmaceutically acceptable salt or solvate thereof, or a tautomer thereof, wherein R₁, R², R⁵, and R⁶ are as defined in connection with Formula I.

In another embodiment, the RTI is a compound is a compound of Formula III, or a pharmaceutically acceptable salt or solvate thereof, or a tautomer thereof, wherein R⁵ is —NH₂. In another embodiment, the RTI is a compound is a compound of Formula III, or a pharmaceutically acceptable salt or solvate thereof, or a tautomer thereof, wherein R⁵ is —OH.

In another embodiment, the RTI is a compound is a compound of Formula III, or a pharmaceutically acceptable salt or solvate thereof, or a tautomer thereof, wherein R⁶ is hydrogen. In another embodiment, the RTI is a compound is a compound of Formula III, or a pharmaceutically acceptable salt or solvate thereof, or a tautomer thereof, wherein R⁶ is chloro. In another embodiment, the RTI is a compound is a compound of Formula III, or a pharmaceutically acceptable salt or solvate thereof, or a tautomer thereof, wherein R⁶ is fluoro. In another embodiment, the RTI is a compound is a compound of Formula III, or a pharmaceutically acceptable salt or solvate thereof, or a tautomer thereof, wherein R⁶ is —NH₂.

In another embodiment, the RTI is a compound is a compound of any one of Formulae I-III, or a pharmaceutically acceptable salt or solvate thereof, or a tautomer thereof, wherein R¹ is hydrogen. In another embodiment, the RTI is a compound is a compound of any one of Formulae I-III, or a pharmaceutically acceptable salt or solvate thereof, or a tautomer thereof, wherein R¹ is —OH.

In another embodiment, the RTI is a compound is a compound of any one of Formulae I-III, or a pharmaceutically acceptable salt or solvate thereof, or a tautomer thereof, wherein R² is methyl. In another embodiment, the RTI is a compound is a compound of any one of Formulae I-III, or a pharmaceutically acceptable salt or solvate thereof, or a tautomer thereof, wherein R² is ethynyl. In another embodiment, the RTI is a compound is a compound of any one of Formulae I-III, or a pharmaceutically acceptable salt or solvate thereof, or a tautomer thereof, wherein R² is —CN.

In another embodiment, the RTI is any one or more of the compounds of Table 3, or a pharmaceutically acceptable salt or solvate thereof, or a tautomer thereof.

TABLE 3

| Cpd. | Structure | Cpd. | Structure |
|---|---|---|---|
| 1 | 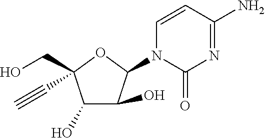 [SMILES: C#C[C@]1(CO)O[C@@H](n2ccc(N)nc2=O)[C@@H](O)[C@@H]1O] | 15 | 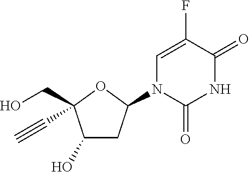 [SMILES: C#C[C@]1(CO)O[C@@H](n2cc(F)c(=O)[nH]c2=O)C[C@@H]1O] |
| 2 | 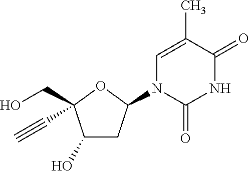 [SMILES: C#C[C@]1(CO)O[C@@H](n2cc(C)c(=O)[nH]c2=O)C[C@@H]1O] | 16 | 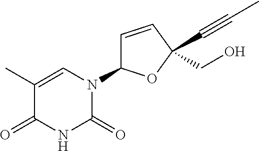 [SMILES: CC#C[C@]1(CO)C=C[C@H](n2cc(C)c(=O)[nH]c2=O)O1] |
| 3 | 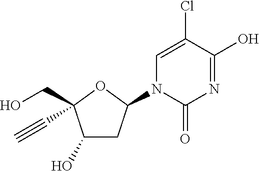 [SMILES: C#C[C@]1(CO)O[C@@H](n2cc(Cl)c(O)nc2=O)C[C@@H]1O] | 17 | 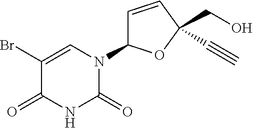 [SMILES: C#C[C@@]1(CO)C=C[C@H](n2cc(Br)c(=O)[nH]c2=O)O1] |
| 4 | 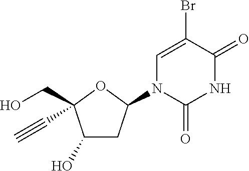 [SMILES: C#C[C@]1(CO)O[C@@H](n2cc(Br)c(=O)[nH]c2=O)C[C@@H]1O] | 18 | 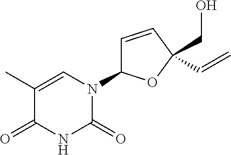 [SMILES: C=C[C@@]1(CO)C=C[C@H](n2cc(C)c(=O)[nH]c2=O)O1] |
| 5 | 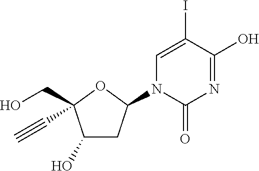 [SMILES: C#C[C@]1(CO)O[C@@H](n2cc(I)c(O)nc2=O)C[C@@H]1O] | 19 | 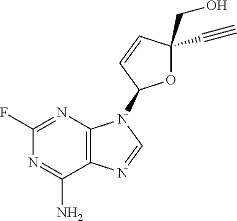 [SMILES: C#C[C@@]1(CO)C=C[C@H](n2cnc3c(N)nc(F)nc32)O1] |
| 6 | 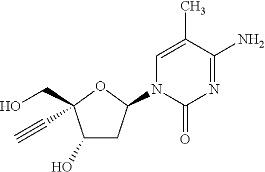 [SMILES: C#C[C@]1(CO)O[C@@H](n2cc(C)c(N)nc2=O)C[C@@H]1O] | 20 | 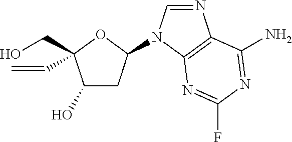 [SMILES: C=C[C@]1(CO)O[C@@H](n2cnc3c(N)nc(F)nc32)C[C@@H]1O] |
| 7 | 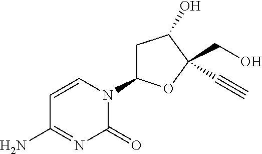 [SMILES: C#C[C@]1(CO)O[C@@H](n2ccc(N)nc2=O)C[C@@H]1O] | 21 | 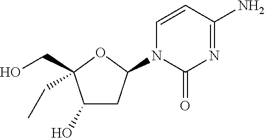 [SMILES: CC[C@]1(CO)O[C@@H](n2ccc(N)nc2=O)C[C@@H]1O] |
| 8 | 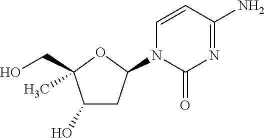 [SMILES: C[C@]1(CO)O[C@@H](n2ccc(N)nc2=O)C[C@@H]1O] | 22 | 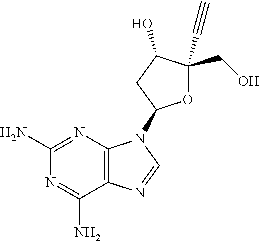 [SMILES: C#C[C@]1(CO)O[C@@H](n2cnc3c(N)nc(N)nc32)C[C@@H]1O] |
| 9 | 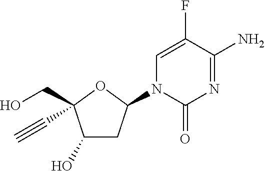 [SMILES: C#C[C@]1(CO)O[C@@H](n2cc(F)c(N)nc2=O)C[C@@H]1O] | 23 | 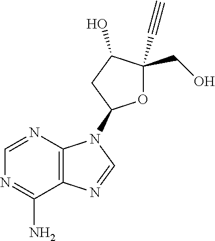 [SMILES: C#C[C@]1(CO)O[C@@H](n2cnc3c(N)ncnc32)C[C@@H]1O] |
| 10 | 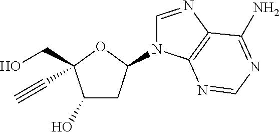 [SMILES: C#C[C@]1(CO)O[C@@H](n2cnc3c(N)ncnc32)C[C@@H]1O] | 24 | 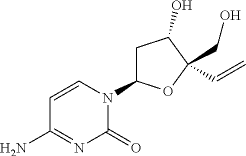 [SMILES: C=C[C@]1(CO)O[C@@H](n2ccc(N)nc2=O)C[C@@H]1O] |
| 11 | 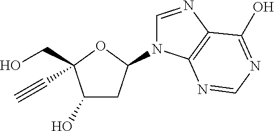 [SMILES: C#C[C@]1(CO)O[C@@H](n2cnc3c(O)ncnc32)C[C@@H]1O] | 25 | 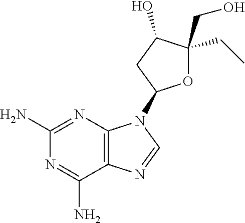 [SMILES: CC[C@]1(CO)O[C@@H](n2cnc3c(N)nc(N)nc32)C[C@@H]1O] |
| 12 | 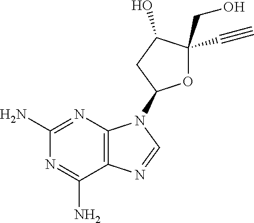 [SMILES: C#C[C@]1(CO)O[C@@H](n2cnc3c(N)nc(N)nc32)C[C@@H]1O] | 26 | 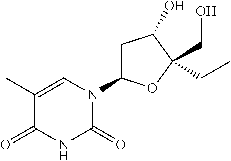 [SMILES: CC[C@]1(CO)O[C@@H](n2cc(C)c(=O)[nH]c2=O)C[C@@H]1O] |
| 13 | 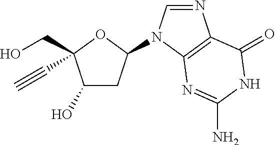 [SMILES: C#C[C@]1(CO)O[C@@H](n2cnc3c(=O)[nH]c(N)nc32)C[C@@H]1O] | 27 | 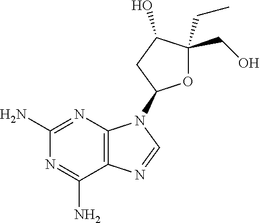 [SMILES: CC[C@]1(CO)O[C@@H](n2cnc3c(N)nc(N)nc32)C[C@@H]1O] |
| 14 | 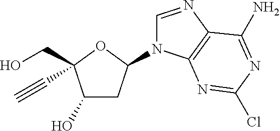 [SMILES: C#C[C@]1(CO)O[C@@H](n2cnc3c(N)nc(Cl)nc32)C[C@@H]1O] | | |

In another embodiment, the RTI is any one or more of the compounds of Table 4, or a pharmaceutically acceptable salt or solvate thereof, or a tautomer thereof.

TABLE 4

| | | |
|---|---|---|
| 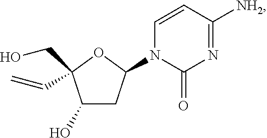 [SMILES: C=C[C@]1(CO)O[C@@H](n2ccc(N)nc2=O)C[C@@H]1O] | 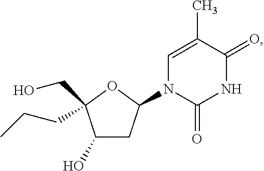 [SMILES: CCC[C@]1(CO)O[C@@H](n2cc(C)c(=O)[nH]c2=O)C[C@@H]1O] | 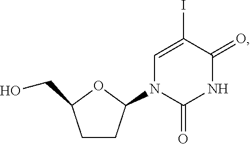 [SMILES: O=c1[nH]c(=O)n([C@H]2CC[C@@H](CO)O2)cc1I] |
| 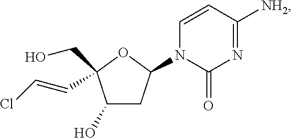 [SMILES: Nc1ccn([C@H]2C[C@H](O)[C@@](/C=C/Cl)(CO)O2)c(=O)n1] | 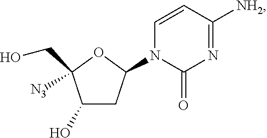 [SMILES: [N-]=[N+]=N[C@]1(CO)O[C@@H](n2ccc(N)nc2=O)C[C@@H]1O] | 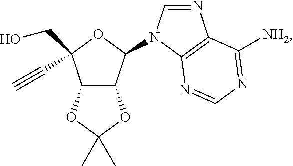 [SMILES: C#C[C@]1(CO)O[C@@H](n2cnc3c(N)ncnc32)[C@@H]2OC(C)(C)O[C@@H]21] |
| 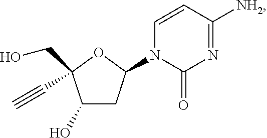 [SMILES: C#C[C@]1(CO)O[C@@H](n2ccc(N)nc2=O)C[C@@H]1O] | 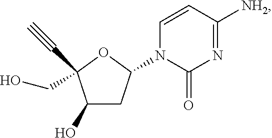 [SMILES: C#C[C@@]1(CO)O[C@H](n2ccc(N)nc2=O)C[C@H]1O] | 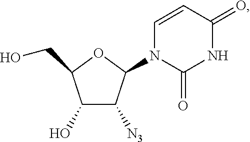 [SMILES: [N-]=[N+]=N[C@@H]1[C@H](O)[C@@H](CO)O[C@H]1n1ccc(=O)[nH]c1=O] |
| 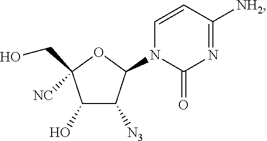 [SMILES: N#C[C@]1(CO)O[C@@H](n2ccc(N)nc2=O)[C@H](N=[N+]=[N-])[C@@H]1O] | 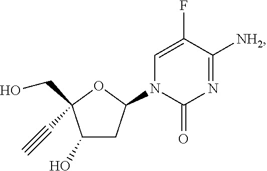 [SMILES: C#C[C@]1(CO)O[C@@H](n2cc(F)c(N)nc2=O)C[C@@H]1O] | 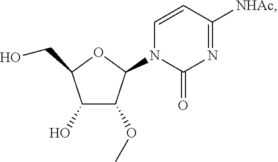 [SMILES: CO[C@@H]1[C@H](O)[C@@H](CO)O[C@H]1n1ccc(NC(C)=O)nc1=O] |
| 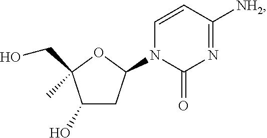 [SMILES: C[C@]1(CO)O[C@@H](n2ccc(N)nc2=O)C[C@@H]1O] | 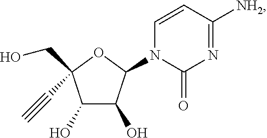 [SMILES: C#C[C@]1(CO)O[C@@H](n2ccc(N)nc2=O)[C@@H](O)[C@@H]1O] | 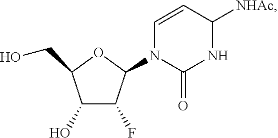 [SMILES: CC(=O)NC1C=CN([C@@H]2O[C@H](CO)[C@@H](O)[C@H]2F)C(=O)N1] |
| 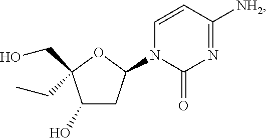 [SMILES: CC[C@]1(CO)O[C@@H](n2ccc(N)nc2=O)C[C@@H]1O] | 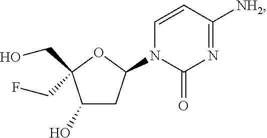 [SMILES: Nc1ccn([C@H]2C[C@H](O)[C@](CO)(CF)O2)c(=O)n1] | 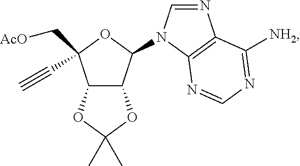 [SMILES: C#C[C@]1(COC(C)=O)O[C@@H](n2cnc3c(N)ncnc32)[C@@H]2OC(C)(C)O[C@@H]21] |
| 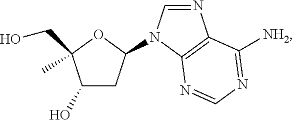 [SMILES: C[C@]1(CO)O[C@@H](n2cnc3c(N)ncnc32)C[C@@H]1O] | 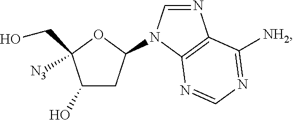 [SMILES: [N-]=[N+]=N[C@]1(CO)O[C@@H](n2cnc3c(N)ncnc32)C[C@@H]1O] | 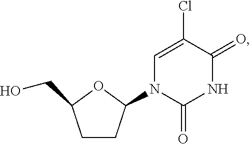 [SMILES: O=c1[nH]c(=O)n([C@H]2CC[C@@H](CO)O2)cc1Cl] |
| 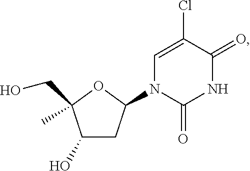 [SMILES: C[C@]1(CO)O[C@@H](n2cc(Cl)c(=O)[nH]c2=O)C[C@@H]1O] | 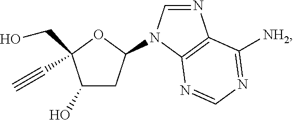 [SMILES: C#C[C@]1(CO)O[C@@H](n2cnc3c(N)ncnc32)C[C@@H]1O] | 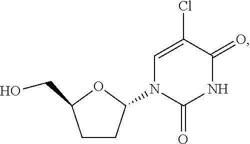 [SMILES: O=c1[nH]c(=O)n([C@@H]2CC[C@@H](CO)O2)cc1Cl] |
| 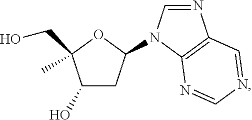 [SMILES: C[C@]1(CO)O[C@@H](n2cnc3cncnc32)C[C@@H]1O] | 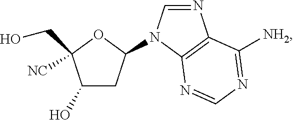 [SMILES: N#C[C@]1(CO)O[C@@H](n2cnc3c(N)ncnc32)C[C@@H]1O] | 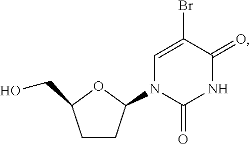 [SMILES: O=c1[nH]c(=O)n([C@H]2CC[C@@H](CO)O2)cc1Br] |
| 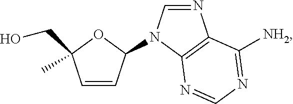 [SMILES: C[C@@]1(CO)C=C[C@H](n2cnc3c(N)ncnc32)O1] | 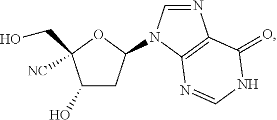 [SMILES: N#C[C@]1(CO)O[C@@H](n2cnc3c(=O)[nH]cnc32)C[C@@H]1O] | 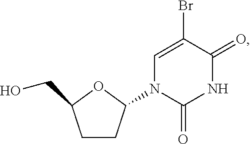 [SMILES: O=c1[nH]c(=O)n([C@@H]2CC[C@@H](CO)O2)cc1Br] |
| 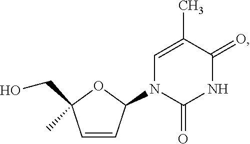 [SMILES: Cc1cn([C@H]2C=C[C@@](C)(CO)O2)c(=O)[nH]c1=O] | 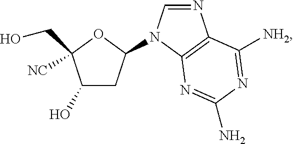 [SMILES: N#C[C@]1(CO)O[C@@H](n2cnc3c(N)nc(N)nc32)C[C@@H]1O] | 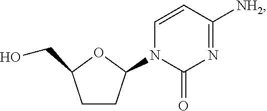 [SMILES: Nc1ccn([C@H]2CC[C@@H](CO)O2)c(=O)n1] |
| 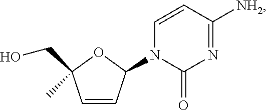 [SMILES: C[C@@]1(CO)C=C[C@H](n2ccc(N)nc2=O)O1] | 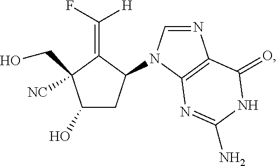 [SMILES: N#C[C@]1(CO)/C(=C\F)[C@@H](n2cnc3c(=O)[nH]c(N)nc32)C[C@@H]1O] | 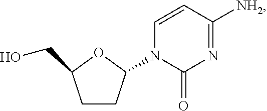 [SMILES: Nc1ccn([C@@H]2CC[C@@H](CO)O2)c(=O)n1] |
| 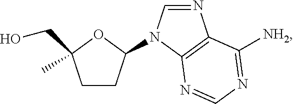 [SMILES: C[C@@]1(CO)CC[C@H](n2cnc3c(N)ncnc32)O1] | 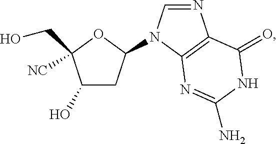 [SMILES: N#C[C@]1(CO)O[C@@H](n2cnc3c(=O)[nH]c(N)nc32)C[C@@H]1O] | 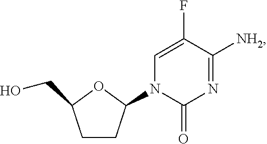 [SMILES: Nc1nc(=O)n([C@H]2CC[C@@H](CO)O2)cc1F] |
| 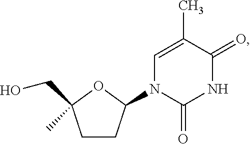 [SMILES: Cc1cn([C@H]2CC[C@@](C)(CO)O2)c(=O)[nH]c1=O] | 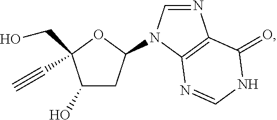 [SMILES: C#C[C@]1(CO)O[C@@H](n2cnc3c(=O)[nH]cnc32)C[C@@H]1O] | 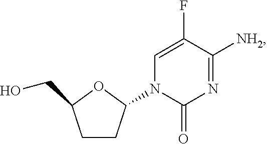 [SMILES: Nc1nc(=O)n([C@@H]2CC[C@@H](CO)O2)cc1F] |
| 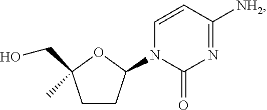 [SMILES: C[C@@]1(CO)CC[C@H](n2ccc(N)nc2=O)O1] | 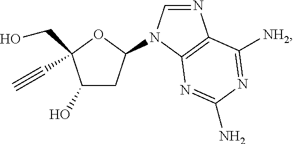 [SMILES: C#C[C@]1(CO)O[C@@H](n2cnc3c(N)nc(N)nc32)C[C@@H]1O] | 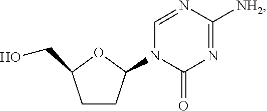 [SMILES: Nc1ncn([C@H]2CC[C@@H](CO)O2)c(=O)n1] |
| 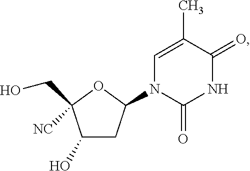 [SMILES: Cc1cn([C@H]2C[C@H](O)[C@@](C#N)(CO)O2)c(=O)[nH]c1=O] | 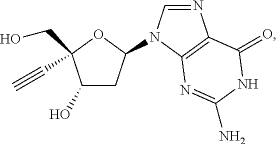 [SMILES: C#C[C@]1(CO)O[C@@H](n2cnc3c(=O)[nH]c(N)nc32)C[C@@H]1O] | 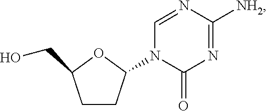 [SMILES: Nc1ncn([C@@H]2CC[C@@H](CO)O2)c(=O)n1] |
| 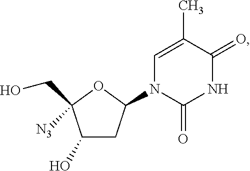 [SMILES: Cc1cn([C@H]2C[C@H](O)[C@](CO)(N=[N+]=[N-])O2)c(=O)[nH]c1=O] | 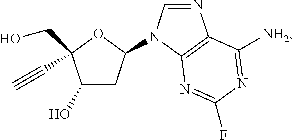 [SMILES: C#C[C@]1(CO)O[C@@H](n2cnc3c(N)nc(F)nc32)C[C@@H]1O] | 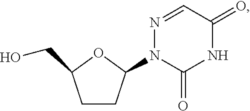 [SMILES: O=c1cnn([C@H]2CC[C@@H](CO)O2)c(=O)[nH]1] |
| 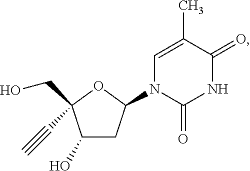 [SMILES: C#C[C@]1(CO)O[C@@H](n2cc(C)c(=O)[nH]c2=O)C[C@@H]1O] | 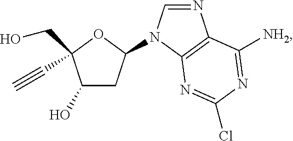 [SMILES: C#C[C@]1(CO)O[C@@H](n2cnc3c(N)nc(Cl)nc32)C[C@@H]1O] | 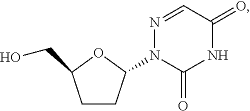 [SMILES: O=c1cnn([C@@H]2CC[C@@H](CO)O2)c(=O)[nH]1] |
| 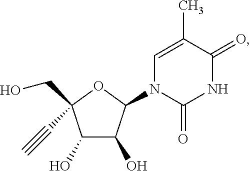 [SMILES: C#C[C@]1(CO)O[C@@H](n2cc(C)c(=O)[nH]c2=O)[C@@H](O)[C@@H]1O] | 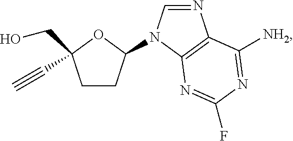 [SMILES: C#C[C@@]1(CO)CC[C@H](n2cnc3c(N)nc(F)nc32)O1] | 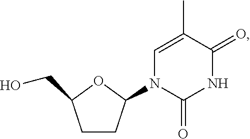 [SMILES: Cc1cn([C@H]2CC[C@@H](CO)O2)c(=O)[nH]c1=O] |
| 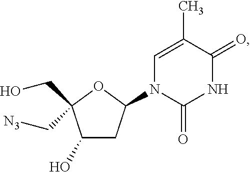 [SMILES: Cc1cn([C@H]2C[C@H](O)[C@](CO)(CN=[N+]=[N-])O2)c(=O)[nH]c1=O] | 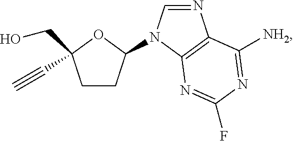 [SMILES: C#C[C@@]1(CO)CC[C@H](n2cnc3c(N)nc(F)nc32)O1] | 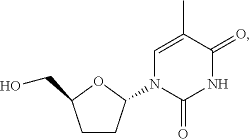 [SMILES: Cc1cn([C@@H]2CC[C@@H](CO)O2)c(=O)[nH]c1=O] |
| 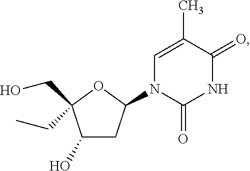 [SMILES: CC[C@]1(CO)O[C@@H](n2cc(C)c(=O)[nH]c2=O)C[C@@H]1O] | 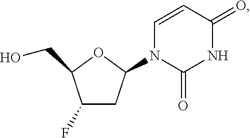 [SMILES: O=c1ccn([C@H]2C[C@H](F)[C@@H](CO)O2)c(=O)[nH]1] | 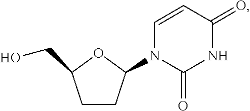 [SMILES: O=c1ccn([C@H]2CC[C@@H](CO)O2)c(=O)[nH]1] |
| 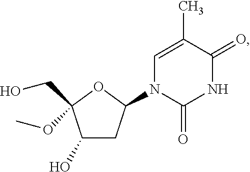 [SMILES: CO[C@]1(CO)O[C@@H](n2cc(C)c(=O)[nH]c2=O)C[C@@H]1O] | 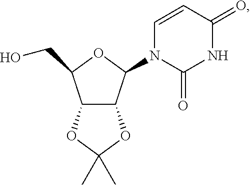 [SMILES: CC1(C)O[C@@H]2[C@H](O1)[C@@H](CO)O[C@H]2n1ccc(=O)[nH]c1=O] | 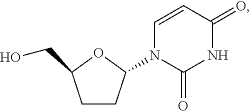 [SMILES: O=c1ccn([C@@H]2CC[C@@H](CO)O2)c(=O)[nH]1] |
| 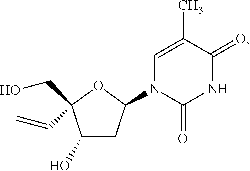 [SMILES: C=C[C@]1(CO)O[C@@H](n2cc(C)c(=O)[nH]c2=O)C[C@@H]1O] | 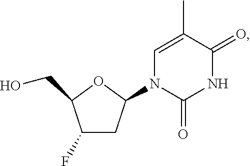 [SMILES: Cc1cn([C@H]2C[C@H](F)[C@@H](CO)O2)c(=O)[nH]c1=O] | 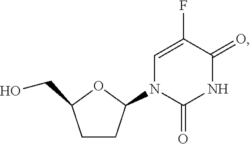 [SMILES: O=c1[nH]c(=O)n([C@H]2CC[C@@H](CO)O2)cc1F] |
| 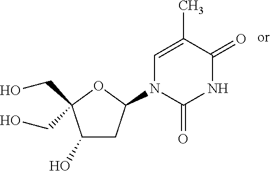 | 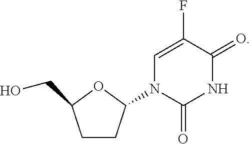 [SMILES: O=c1[nH]c(=O)n([C@@H]2CC[C@@H](CO)O2)cc1F] | |

In another embodiment, the RTI is a compound of Table 4, or a pharmaceutically acceptable salt or solvate thereof, or a tautomer thereof, selected from the group consisting of:

| Cpd. | Structure |
|---|---|
| 29 | 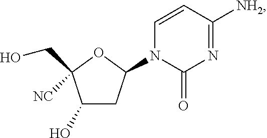 [SMILES: N#C[C@]1(CO)O[C@@H](n2ccc(N)nc2=O)C[C@@H]1O] |
| 30 | 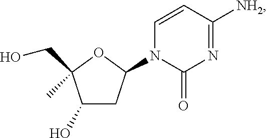 [SMILES: C[C@]1(CO)O[C@@H](n2ccc(N)nc2=O)C[C@@H]1O] |
| 31 | 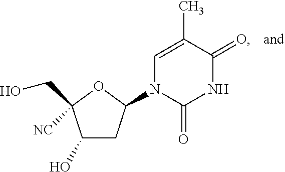 [SMILES: Cc1cn([C@H]2C[C@H](O)[C@@](C#N)(CO)O2)c(=O)[nH]c1=O ; "and"] |
| 32 | 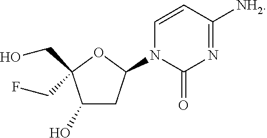 [SMILES: Nc1ccn([C@H]2C[C@H](O)[C@](CO)(CF)O2)c(=O)n1] |

The compounds of Formulae I-III, Table 3, and Table 4 may be found and prepared as described, for example, in Nomura et al., J. Med. Chem. 42:2901-2908 (1999); Ohrui et al., J. Med. Chem. 43:4516-4525 (2000); Ohrui, H., Proc. Jpn. Acad. Ser. B 87:53-65 (2011); Banuelos-Sanchez et al., Cell Chemical Biology 26:1095-1109 (2019); Kirby et al., Antimicrobial Agents and Chemotherapy 57:6254-6264 (2013); Kodama et al., Antimicrobial Agents and Chemotherapy 45:1539-1546 (2013); Higashi-Kuwata et al., Journal of Hepatology 74:1075-1086 (2021); JP Patent No. 6767011; U.S. Pat. No. 10,933,067; and/or as described in EXAMPLES 4-6, below.

In some embodiments, provided is a method for treating cancer, e.g., breast, colon, lung, pancreatic ductal, prostate, ovarian, or head and neck cancer, in patient in need thereof comprising administering a therapeutically effective amount of a compound of Table 3 or Table 4, e.g., Cpd. No. 7, Cpd. No. 9, Cpd. No. 13, Cpd. No. 15, Cpd. No. 20, Cpd. No. 21, Cpd. No. 25, Cpd. No. 25, Cpd. No. 30, Cpd. No. 31, Cpd. No. 32, islatravir, or elvucitabine, to the patient.

The term “tautomer” as used herein refers to each of two or more isomers of a compound which exist together in equilibrium, and are interchanged by migration of an atom, e.g., a hydrogen, or group within the molecule. Certain compounds of the disclosure may exist as tautomers.

In some embodiments, provided is a method for treating cancer, e.g., breast, colon, lung, pancreatic ductal, prostate, ovarian, or head and neck cancer, by administering elvucitabine a patient in need thereof. Elvucitabine is a compound having the following chemical structure:

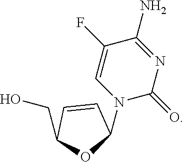 [SMILES: Nc1nc(=O)n([C@@H]2C=C[C@H](CO)O2)cc1F]

Elvucitabine and its method of synthesis is described in U.S. Pat. No. 5,627,160. In some embodiments, elvucitabine is administered to the subject according to an intermittent dosing schedule. In some embodiments, elvucitabine is administered to the subject according to a continuous dosing schedule

The term “LINE-1 inhibitor” as used herein refers to a compound that inhibits human LINE-1 retrotransposition, e.g., with a half maximal inhibitory concentration (IC₅₀) of about 50 μM or less in a HeLa cell-based dual-luciferase assay as described in EXAMPLE 1, see below. See also Jones et al., (2008) PLoS ONE 3(2): e1547. doi:10.1371/journal.pone.0001547; Xie et al., (2011) Nucleic Acids Res. 39(3): e16. doi: 10.1093/nar/gkq1076. In another embodiment, the IC₅₀ is 1 μM or less. In another embodiment, the IC₅₀ is 0.5 μM or less. In another embodiment, the IC₅₀ is 0.25 μM or less. In another embodiment, the IC₅₀ is 0.15 μM or less. In another embodiment, the IC₅₀ is 0.1 μM or less. In another embodiment, the IC₅₀ is 0.05 μM or less. In another embodiment, the IC₅₀ is 0.01 μM or less. In another embodiment, the IC₅₀ is 0.005 μM or less. In some embodiments, the LINE-1 inhibitor is also a nucleoside reverse transcriptase inhibitor (NRTI). LINE-1 inhibitors are described, for example, in WO 2020/154656. Non-limiting exemplary LINE-1 inhibitors include islatravir, elvucitabine, lamivudine (3TC), zidovudine (AZT), tenofovir, tenofovir disoproxil, tenofovir alaphenamide, stavudine (d4T), zalcitabine (ddC), didanosine (ddI), emtricitabine (FTC), entecavir (ETV), 2′,3′-dideoxyguanosine (ddG), 2′,3′-dideoxyadenosine (ddA), 2′-fluoro-2′,3′-dideoxyarabinosyladenine (F-ddA), efavirenz (EFV), nevirapine (NVP), and abacavir (ABC).

The term “adjuvant therapy” as used herein refers to the treatment of cancer during or after a surgical intervention, radiotherapy, chemotherapy, and/or hormone therapy.

The term “neoadjuvant therapy” as used herein refers to the treatment of cancer prior to a surgical intervention, radiotherapy, chemotherapy, and/or hormone therapy. See, e.g., Kent and Hussain, Rev. Urol. 5(suppl 3):S28-S37 (2003). The object of neoadjuvant cancer therapy is to reduce the size or extent of the patient's tumor(s) before the primary therapy, preferably improving the likelihood of successful outcome and/or decreasing the adverse effects of more extensive treatment that would be required in the absence of neoadjuvant therapy.

There are multiple definitions used to categorize patients with high-risk prostate cancer. Pretreatment parameters, including clinical stage, prostate-specific antigen (PSA), and Gleason score, are established predictors of disease recurrence and have historically been used in high-risk disease classifications. See, e.g., McKay et al, Soc Clin Oncol Ed Book 40 (2020) e241-e252. The term “high-risk localized prostate cancer” as used herein refers to prostate cancer classified as clinical T stage cT3a with a Gleason score of at least 8 and/or a PSA of at least 20 ng/mL.

The terms “intermittent dose administration,” “intermittent dosing schedule,” and similar terms as used herein refer to non-continuous administration of a RTI to a subject. Intermittent dose administration regimens useful in the present disclosure encompass any discontinuous administration regimen that provides a therapeutically effective amount of a RTI to a subject in need thereof. Intermittent dosing regimens can use equivalent, lower, or higher doses of a RTI than would be used in continuous dosing regimens. Advantages of intermittent dose administration include, but are not limited to, improved safety, decreased toxicity, e.g., decreased weight loss, increased exposure, increased efficacy, and/or increased subject compliance. These advantages may be realized when a RTI is administered as a single agent or when administered in combination with one or more additional therapeutic agents, e.g., a STING agonist.

In one embodiment, the RTI is administered to the subject every other day.

In another embodiment, the RTI is administered to the subject once a week.

In another embodiment, the RTI is administered to the subject twice a week on consecutive days, e.g., on Monday and Tuesday.

In another embodiment, the RTI is administered to the subject twice a week on non-consecutive days, e.g., on Monday and Wednesday.

In another embodiment, the RTI is administered to the subject three times a week on consecutive days, e.g., on Monday, Tuesday, and Wednesday.

In another embodiment, the RTI is administered to the subject three times a week on non-consecutive days, e.g., on Monday, Wednesday, and Friday.

In one embodiment, the RTI is administered to the subject for about consecutive 4 weeks in a row followed by 1 day or 2, 3, 4, 5, 6, or 7 consecutive days in a row wherein the RTI is not administered to the subject.

In one embodiment, the RTI is administered to the subject for about 3 consecutive weeks in a row followed by 1 day or 2, 3, 4, 5, 6, or 7 consecutive days in a row wherein the RTI is not administered to the subject.

In one embodiment, the RTI is administered to the subject for about 2 consecutive weeks in a row followed by 1 day or 2, 3, 4, 5, 6, or 7 consecutive days in a row wherein the RTI is not administered to the subject.

In one embodiment, the RTI is administered to the subject for 3 consecutive weeks in a row followed by 1 day or 2, 3, 4, or 5 consecutive days in a row wherein the RTI is not administered to the subject

In one embodiment, the RTI is administered to the subject for 2, 3, 4, 5, 6, 7, 8, 9, 10, 11, 12, 13, or 14 consecutive days in a row followed by 1 day or 2, 3, 4, or 5 consecutive days in a row wherein the RTI is not administered to the subject.

In one embodiment, the RTI is administered to the subject for 2, 3, 4, 5, 6, 7, 8, 9, 10, 11, 12, 13, or 14 consecutive days in a row followed by 1 day or 2, 3, or 4 consecutive days in a row wherein the RTI is not administered to the subject.

In one embodiment, the RTI is administered to the subject for 2, 3, 4, 5, 6, 7, 8, 9, 10, 11, 12, 13, or 14 consecutive days in a row followed by about 7, 14, 21, or 28 consecutive days in a row wherein the RTI is not administered to the subject.

In one embodiment, the RTI is administered to the subject for 2, 3, 4, 5, 6, 7, 8, 9, or 10 consecutive days in a row followed by 1 day or 2, 3, or 4 consecutive days in a row wherein the RTI is not administered to the subject.

In another embodiment, the RTI is administered to the subject for 2 consecutive days in a row followed by 3 or 4 consecutive days in a row wherein the RTI is not administered.

In another embodiment, the RTI is administered to the subject for 3 consecutive days in a row followed by 3 or 4 days in a row wherein the RTI is not administered.

In another embodiment, the RTI is administered to the subject for 4 consecutive days in a row followed by 3 or 4 consecutive days in a row wherein the RTI is not administered.

In another embodiment, the RTI is administered to the subject for 5 consecutive days in a row followed by 3 or 4 consecutive days in a row wherein the RTI is not administered.

In another embodiment, the RTI is administered to the subject for 6 consecutive days in a row followed by 3 or 4 consecutive days in a row wherein the RTI is not administered.

In another embodiment, the RTI is administered to the subject for 7 consecutive days in a row followed by 3 or 4 consecutive days in a row wherein the RTI is not administered.

In another embodiment, the RTI is administered to the subject for 8 consecutive days in a row followed by 3 or 4 consecutive days in a row wherein the RTI is not administered.

In another embodiment, the RTI is administered to the subject for 9 consecutive days in a row followed by 3 or 4 consecutive days in a row wherein the RTI is not administered.

In another embodiment, the RTI is administered to the subject for 10 consecutive days in a row followed by 3 or 4 consecutive days in a row wherein the RTI is not administered.

In another embodiment, the RTI is administered to the subject for 11 consecutive days in a row followed by 3 or 4 consecutive days in a row wherein the RTI is not administered.

In another embodiment, the RTI is administered to the subject for 12 consecutive days in a row followed by 3 or 4 consecutive days in a row wherein the RTI is not administered.

In another embodiment, the RTI is administered to the subject for 13 consecutive days in a row followed by 3 or 4 consecutive days in a row wherein the RTI is not administered.

In another embodiment, the RTI is administered to the subject for 14 consecutive days in a row followed by 3 or 4 consecutive days in a row wherein the RTI is not administered.

In another embodiment, the RTI is administered to the subject for 15 consecutive days in a row followed by 3 or 4 consecutive days in a row wherein the RTI is not administered.

In another embodiment, the RTI is administered to the subject for 16 consecutive days in a row followed by 3 or 4 consecutive days in a row wherein the RTI is not administered.

In another embodiment, the RTI is administered to the subject for 17 consecutive days in a row followed by 3 or 4 consecutive days in a row wherein the RTI is not administered.

In another embodiment, the RTI is administered to the subject for 18 consecutive days in a row followed by 3 or 4 consecutive days in a row wherein the RTI is not administered.

In another embodiment, the RTI is administered to the subject for 19 consecutive days in a row followed by 3 or 4 consecutive days in a row wherein the RTI is not administered.

In another embodiment, the RTI is administered to the subject for 20 consecutive days in a row followed by 3 or 4 consecutive days in a row wherein the RTI is not administered.

In another embodiment, the RTI is administered to the subject for 21 consecutive days in a row followed by days 3 or 4 consecutive in a row wherein the RTI is not administered.

In another embodiment, the RTI is administered to the subject for 2 consecutive days in a row followed by 2 or 3 consecutive days in a row wherein the RTI is not administered.

In another embodiment, the RTI is administered to the subject for 3 consecutive days in a row followed by 2 or 3 consecutive days in a row wherein the RTI is not administered.

In another embodiment, the RTI is administered to the subject for 4 consecutive days in a row followed by 2 or 3 consecutive days in a row wherein the RTI is not administered.

In another embodiment, the RTI is administered to the subject for 5 consecutive days in a row followed by 2 or 3 consecutive days in a row wherein the RTI is not administered.

In another embodiment, the RTI is administered to the subject for 6 consecutive days in a row followed by 2 or 3 consecutive days in a row wherein the RTI is not administered.

In another embodiment, the RTI is administered to the subject for 7 consecutive days in a row followed by 2 or 3 consecutive days in a row wherein the RTI is not administered.

In another embodiment, the RTI is administered to the subject for 8 consecutive days in a row followed by 2 or 3 consecutive days in a row wherein the RTI is not administered.

In another embodiment, the RTI is administered to the subject for 9 consecutive days in a row followed by 2 or 3 consecutive days in a row wherein the RTI is not administered.

In another embodiment, the RTI is administered to the subject for 10 consecutive days in a row followed by 2 or 3 consecutive days in a row wherein the RTI is not administered.

In another embodiment, the RTI is administered to the subject for 11 consecutive days in a row followed by 2 or 3 consecutive days in a row wherein the RTI is not administered.

In another embodiment, the RTI is administered to the subject for 12 consecutive days in a row followed by 2 or 3 consecutive days in a row wherein the RTI is not administered.

In another embodiment, the RTI is administered to the subject for 13 consecutive days in a row followed by 2 or 3 consecutive days in a row wherein the RTI is not administered.

In another embodiment, the RTI is administered to the subject for 14 consecutive days in a row followed by 2 or 3 consecutive days in a row wherein the RTI is not administered.

In another embodiment, the RTI is administered to the subject for 15 consecutive days in a row followed by 2 or 3 consecutive days in a row wherein the RTI is not administered.

In another embodiment, the RTI is administered to the subject for 16 consecutive days in a row followed by 2 or 3 consecutive days in a row wherein the RTI is not administered.

In another embodiment, the RTI is administered to the subject for 17 consecutive days in a row followed by 2 or 3 consecutive days in a row wherein the RTI is not administered.

In another embodiment, the RTI is administered to the subject for 18 consecutive days in a row followed by 2 or 3 consecutive days in a row wherein the RTI is not administered.

In another embodiment, the RTI is administered to the subject for 19 consecutive days in a row followed by 2 or 3 consecutive days in a row wherein the RTI is not administered.

In another embodiment, the RTI is administered to the subject for 20 consecutive days in a row followed by 2 or 3 consecutive days in a row wherein the RTI is not administered.

In another embodiment, the RTI is administered to the subject for 21 consecutive days in a row followed by 2 or 3 consecutive days in a row wherein the RTI is not administered.

In another embodiment, the RTI is administered to the subject for 2 consecutive days in a row followed by 1 day or 2 consecutive days in a row wherein the RTI is not administered.

In another embodiment, the RTI is administered to the subject for 3 consecutive days in a row followed by 1 day or 2 consecutive days in a row wherein the RTI is not administered.

In another embodiment, the RTI is administered to the subject for 4 consecutive days in a row followed by 1 day or 2 consecutive days in a row wherein the RTI is not administered.

In another embodiment, the RTI is administered to the subject for 5 consecutive days in a row followed by 1 day or 2 consecutive days in a row wherein the RTI is not administered.

In another embodiment, the RTI is administered to the subject for 5 consecutive days in a row followed by 2 consecutive days in a row wherein the RTI is not administered.

In another embodiment, the RTI is administered to the subject for 6 consecutive days in a row followed by 1 day or 2 consecutive days in a row wherein the RTI is not administered.

In another embodiment, the RTI is administered to the subject for 7 consecutive days in a row followed by 1 day or 2 consecutive days in a row wherein the RTI is not administered.

In another embodiment, the RTI is administered to the subject for 8 consecutive days in a row followed by 1 day or 2 consecutive days in a row wherein the RTI is not administered.

In another embodiment, the RTI is administered to the subject for 9 consecutive days in a row followed by 1 day or 2 consecutive days in a row wherein the RTI is not administered.

In another embodiment, the RTI is administered to the subject for 10 consecutive days in a row followed by 1 day or 2 consecutive days in a row wherein the RTI is not administered.

In another embodiment, the RTI is administered to the subject for 11 consecutive days in a row followed by 1 day or 2 consecutive days in a row wherein the RTI is not administered.

In another embodiment, the RTI is administered to the subject for 12 consecutive days in a row followed by 1 day or 2 consecutive days in a row wherein the RTI is not administered.

In another embodiment, the RTI is administered to the subject for 13 consecutive days in a row followed by 1 day or 2 consecutive days in a row wherein the RTI is not administered.

In another embodiment, the RTI is administered to the subject for 14 consecutive days in a row followed by 1 day or 2 consecutive days in a row wherein the RTI is not administered.

In another embodiment, the RTI is administered to the subject for 15 consecutive days in a row followed by 1 day or 2 consecutive days in a row wherein the RTI is not administered.

In another embodiment, the RTI is administered to the subject for 16 consecutive days in a row followed by 1 day or 2 consecutive days in a row wherein the RTI is not administered.

In another embodiment, the RTI is administered to the subject for 17 consecutive days in a row followed by 1 day or 2 consecutive days in a row wherein the RTI is not administered.

In another embodiment, the RTI is administered to the subject for 18 consecutive days in a row followed by 1 day or 2 consecutive days in a row wherein the RTI is not administered.

In another embodiment, the RTI is administered to the subject for 19 consecutive days in a row followed by 1 day or 2 consecutive days in a row wherein the RTI is not administered.

In another embodiment, the RTI is administered to the subject for 20 consecutive days in a row followed by 1 day or 2 consecutive days in a row wherein the RTI is not administered.

In another embodiment, the RTI is administered to the subject for 21 consecutive days in a row followed by 1 day or 2 consecutive days in a row wherein the RTI is not administered.

In another embodiment, the RTI is administered to the subject for 2 consecutive days in a row followed by about 1 consecutive week wherein the RTI is not administered.

In another embodiment, the RTI is administered to the subject for 3 consecutive days in a row followed by about 1 consecutive week wherein the RTI is not administered.

In another embodiment, the RTI is administered to the subject for 4 consecutive days in a row followed by about 1 consecutive week wherein the RTI is not administered.

In another embodiment, the RTI is administered to the subject for 5 consecutive days in a row followed by about 1 consecutive week wherein the RTI is not administered.

In another embodiment, the RTI is administered to the subject for 5 consecutive days in a row followed by 2 consecutive days in a row wherein the RTI is not administered.

In another embodiment, the RTI is administered to the subject for 6 consecutive days in a row followed by about 1 consecutive week wherein the RTI is not administered.

In another embodiment, the RTI is administered to the subject for 7 consecutive days in a row followed by about 1 consecutive week wherein the RTI is not administered.

In another embodiment, the RTI is administered to the subject for 8 consecutive days in a row followed by about 1 consecutive week wherein the RTI is not administered.

In another embodiment, the RTI is administered to the subject for 9 consecutive days in a row followed by about 1 consecutive week wherein the RTI is not administered.

In another embodiment, the RTI is administered to the subject for 10 consecutive days in a row followed by about 1 consecutive week wherein the RTI is not administered.

In another embodiment, the RTI is administered to the subject for 11 consecutive days in a row followed by about 1 consecutive week wherein the RTI is not administered.

In another embodiment, the RTI is administered to the subject for 12 consecutive days in a row followed by about 1 consecutive week wherein the RTI is not administered.

In another embodiment, the RTI is administered to the subject for 13 consecutive days in a row followed by about 1 consecutive week wherein the RTI is not administered.

In another embodiment, the RTI is administered to the subject for 14 consecutive days in a row followed by about 1 consecutive week wherein the RTI is not administered.

In another embodiment, the RTI is administered to the subject for 15 consecutive days in a row followed by about 1 consecutive week wherein the RTI is not administered.

In another embodiment, the RTI is administered to the subject for 16 consecutive days in a row followed by about 1 consecutive week wherein the RTI is not administered.

In another embodiment, the RTI is administered to the subject for 17 consecutive days in a row followed by about 1 consecutive week wherein the RTI is not administered.

In another embodiment, the RTI is administered to the subject for 18 consecutive days in a row followed by about 1 consecutive week wherein the RTI is not administered.

In another embodiment, the RTI is administered to the subject for 19 consecutive days in a row followed by about 1 consecutive week wherein the RTI is not administered.

In another embodiment, the RTI is administered to the subject for 20 consecutive days in a row followed by about 1 consecutive week wherein the RTI is not administered.

In another embodiment, the RTI is administered to the subject for 21 consecutive days in a row followed by about 1 consecutive week wherein the RTI is not administered.

In another embodiment, the RTI is administered to the subject for 2 consecutive days in a row followed by about 2 consecutive weeks in a row wherein the RTI is not administered.

In another embodiment, the RTI is administered to the subject for 3 consecutive days in a row followed by about 2 consecutive weeks in a row wherein the RTI is not administered.

In another embodiment, the RTI is administered to the subject for 4 consecutive days in a row followed by about 2 consecutive weeks in a row wherein the RTI is not administered.

In another embodiment, the RTI is administered to the subject for 5 consecutive days in a row followed by about 2 consecutive weeks in a row wherein the RTI is not administered.

In another embodiment, the RTI is administered to the subject for 5 consecutive days in a row followed by 2 consecutive days in a row wherein the RTI is not administered.

In another embodiment, the RTI is administered to the subject for 6 consecutive days in a row followed by about 2 consecutive weeks in a row wherein the RTI is not administered.

In another embodiment, the RTI is administered to the subject for 7 consecutive days in a row followed by about 2 consecutive weeks in a row wherein the RTI is not administered.

In another embodiment, the RTI is administered to the subject for 8 consecutive days in a row followed by about 2 consecutive weeks in a row wherein the RTI is not administered.

In another embodiment, the RTI is administered to the subject for 9 consecutive days in a row followed by about 2 consecutive weeks in a row wherein the RTI is not administered.

In another embodiment, the RTI is administered to the subject for 10 consecutive days in a row followed by about 2 consecutive weeks in a row wherein the RTI is not administered.

In another embodiment, the RTI is administered to the subject for 11 consecutive days in a row followed by about 2 consecutive weeks in a row wherein the RTI is not administered.

In another embodiment, the RTI is administered to the subject for 12 consecutive days in a row followed by about 2 consecutive weeks in a row wherein the RTI is not administered.

In another embodiment, the RTI is administered to the subject for 13 consecutive days in a row followed by about 2 consecutive weeks in a row wherein the RTI is not administered.

In another embodiment, the RTI is administered to the subject for 14 consecutive days in a row followed by about 2 consecutive weeks in a row wherein the RTI is not administered.

In another embodiment, the RTI is administered to the subject for 15 consecutive days in a row followed by about 2 consecutive weeks in a row wherein the RTI is not administered.

In another embodiment, the RTI is administered to the subject for 16 consecutive days in a row followed by about 2 consecutive weeks in a row wherein the RTI is not administered.

In another embodiment, the RTI is administered to the subject for 17 consecutive days in a row followed by about 2 consecutive weeks in a row wherein the RTI is not administered.

In another embodiment, the RTI is administered to the subject for 18 consecutive days in a row followed by about 2 consecutive weeks in a row wherein the RTI is not administered.

In another embodiment, the RTI is administered to the subject for 19 consecutive days in a row followed by about 2 consecutive weeks in a row wherein the RTI is not administered.

In another embodiment, the RTI is administered to the subject for 20 consecutive days in a row followed by about 2 consecutive weeks in a row wherein the RTI is not administered.

In another embodiment, the RTI is administered to the subject for 21 consecutive days in a row followed by about 2 consecutive weeks in a row wherein the RTI is not administered.

In another embodiment, the RTI is administered to the subject for 2 consecutive days in a row followed by about 4 consecutive weeks in a row wherein the RTI is not administered.

In another embodiment, the RTI is administered to the subject for 3 consecutive days in a row followed by about 4 consecutive weeks in a row wherein the RTI is not administered.

In another embodiment, the RTI is administered to the subject for 4 consecutive days in a row followed by about 4 consecutive weeks in a row wherein the RTI is not administered.

In another embodiment, the RTI is administered to the subject for 5 consecutive days in a row followed by about 4 consecutive weeks in a row wherein the RTI is not administered.

In another embodiment, the RTI is administered to the subject for 5 consecutive days in a row followed by 4 consecutive days in a row wherein the RTI is not administered.

In another embodiment, the RTI is administered to the subject for 6 consecutive days in a row followed by about 4 consecutive weeks in a row wherein the RTI is not administered.

In another embodiment, the RTI is administered to the subject for 7 consecutive days in a row followed by about 4 consecutive weeks in a row wherein the RTI is not administered.

In another embodiment, the RTI is administered to the subject for 8 consecutive days in a row followed by about 4 consecutive weeks in a row wherein the RTI is not administered.

In another embodiment, the RTI is administered to the subject for 9 consecutive days in a row followed by about 4 consecutive weeks in a row wherein the RTI is not administered.

In another embodiment, the RTI is administered to the subject for 10 consecutive days in a row followed by about 4 consecutive weeks in a row wherein the RTI is not administered.

In another embodiment, the RTI is administered to the subject for 11 consecutive days in a row followed by about 4 consecutive weeks in a row wherein the RTI is not administered.

In another embodiment, the RTI is administered to the subject for 14 consecutive days in a row followed by about 4 consecutive weeks in a row wherein the RTI is not administered.

In another embodiment, the RTI is administered to the subject for 13 consecutive days in a row followed by about 4 consecutive weeks in a row wherein the RTI is not administered.

In another embodiment, the RTI is administered to the subject for 14 consecutive days in a row followed by about 4 consecutive weeks in a row wherein the RTI is not administered.

In another embodiment, the RTI is administered to the subject for 15 consecutive days in a row followed by about 4 consecutive weeks in a row wherein the RTI is not administered.

In another embodiment, the RTI is administered to the subject for 16 consecutive days in a row followed by about 4 consecutive weeks in a row wherein the RTI is not administered.

In another embodiment, the RTI is administered to the subject for 17 consecutive days in a row followed by about 4 consecutive weeks in a row wherein the RTI is not administered.

In another embodiment, the RTI is administered to the subject for 18 consecutive days in a row followed by about 4 consecutive weeks in a row wherein the RTI is not administered.

In another embodiment, the RTI is administered to the subject for 19 consecutive days in a row followed by about 4 consecutive weeks in a row wherein the RTI is not administered.

In another embodiment, the RTI is administered to the subject for 20 consecutive days in a row followed by about 4 consecutive weeks in a row wherein the RTI is not administered.

In another embodiment, the RTI is administered to the subject for 21 consecutive days in a row followed by about 4 consecutive weeks in a row wherein the RTI is not administered.

In another embodiment, the RTI is administered to the subject for 2 consecutive days in a row followed by about 4 consecutive weeks in a row wherein the RTI is not administered.

In another embodiment, the RTI is administered to the subject for 3 consecutive days in a row followed by about 4 consecutive weeks in a row wherein the RTI is not administered.

In another embodiment, the RTI is administered to the subject for 4 consecutive days in a row followed by about 4 consecutive weeks in a row wherein the RTI is not administered.

In another embodiment, the RTI is administered to the subject for 5 consecutive days in a row followed by about 4 consecutive weeks in a row wherein the RTI is not administered.

In another embodiment, the RTI is administered to the subject for 5 consecutive days in a row followed by 3 consecutive days in a row wherein the RTI is not administered.

In another embodiment, the RTI is administered to the subject for 6 consecutive days in a row followed by about 4 consecutive weeks in a row wherein the RTI is not administered.

In another embodiment, the RTI is administered to the subject for 7 consecutive days in a row followed by about 4 consecutive weeks in a row wherein the RTI is not administered.

In another embodiment, the RTI is administered to the subject for 7 consecutive days in a row followed by about 8 consecutive weeks in a row wherein the RTI is not administered.

In another embodiment, the RTI is administered to the subject for 8 consecutive days in a row followed by about 4 consecutive weeks in a row wherein the RTI is not administered.

In another embodiment, the RTI is administered to the subject for 9 consecutive days in a row followed by about 4 consecutive weeks in a row wherein the RTI is not administered.

In another embodiment, the RTI is administered to the subject for 10 consecutive days in a row followed by about 4 consecutive weeks in a row wherein the RTI is not administered.

In another embodiment, the RTI is administered to the subject for 11 consecutive days in a row followed by about 4 consecutive weeks in a row wherein the RTI is not administered.

In another embodiment, the RTI is administered to the subject for 13 consecutive days in a row followed by about 4 consecutive weeks in a row wherein the RTI is not administered.

In another embodiment, the RTI is administered to the subject for 13 consecutive days in a row followed by about 4 consecutive weeks in a row wherein the RTI is not administered.

In another embodiment, the RTI is administered to the subject for 14 consecutive days in a row followed by about 4 consecutive weeks in a row wherein the RTI is not administered.

In another embodiment, the RTI is administered to the subject for 15 consecutive days in a row followed by about 4 consecutive weeks in a row wherein the RTI is not administered.

In another embodiment, the RTI is administered to the subject for 16 consecutive days in a row followed by about 4 consecutive weeks in a row wherein the RTI is not administered.

In another embodiment, the RTI is administered to the subject for 17 consecutive days in a row followed by about 4 consecutive weeks in a row wherein the RTI is not administered.

In another embodiment, the RTI is administered to the subject for 18 consecutive days in a row followed by about 4 consecutive weeks in a row wherein the RTI is not administered.

In another embodiment, the RTI is administered to the subject for 19 consecutive days in a row followed by about 4 consecutive weeks in a row wherein the RTI is not administered.

In another embodiment, the RTI is administered to the subject for 20 consecutive days in a row followed by about 4 consecutive weeks in a row wherein the RTI is not administered.

In another embodiment, the RTI is administered to the subject for 21 consecutive days in a row followed by about 4 consecutive weeks in a row wherein the RTI is not administered.

In another embodiment, the RTI is administered to the subject for about 2 consecutive weeks in a row followed by about 12 consecutive weeks in a row wherein the RTI is not administered.

In another embodiment, the RTI is administered to the subject for about 3 consecutive weeks in a row followed by about 12 consecutive weeks in a row wherein the RTI is not administered.

In another embodiment, the RTI is administered to the subject for about 4 consecutive weeks in a row followed by about 12 consecutive weeks in a row wherein the RTI is not administered.

Examples of treatable cancers include, but are not limited to, any one or more of the cancers of Table 1.

TABLE 1

| | | | |
|---|---|---|---|
| adrenal cancer | acinic cell carcinoma | acoustic neuroma | acral lentigious |
| | | | melanoma |
| acrospiroma | acute eosinophilic | acute erythroid | acute lymphoblastic |
| | leukemia | leukemia | leukemia |
| acute | acute monocytic | acute promyelocytic | adenocarcinoma |
| megakaryoblastic | leukemia | leukemia | |
| leukemia | | | |
| adenoid cystic | adenoma | adenomatoid | adenosquamous |
| carcinoma | | odontogenic tumor | carcinoma |
| adipose tissue | adrenocortical | adult T-cell | aggressive NK-cell |
| neoplasm | carcinoma | leukemia/lymphoma | leukemia |
| AIDS-related | alveolar | alveolar soft part | ameloblastic fibroma |
| lymphoma | rhabdomyosarcoma | sarcoma | |
| anaplastic large cell | anaplastic thyroid | angioimmunoblastic | angiomyolipoma |
| lymphoma | cancer | T-cell lymphoma | |
| angiosarcoma | astrocytoma | atypical teratoid | B-cell chronic |
| | | rhabdoid tumor | lymphocytic |
| | | | leukemia |
| B-cell | B-cell lymphoma | basal cell carcinoma | biliary tract cancer |
| prolymphocytic | | | |
| leukemia | | | |
| bladder cancer | blastoma | bone cancer | Brenner tumor |
| Brown tumor | Burkitt's lymphoma | breast cancer | brain cancer |
| carcinoma | carcinoma in situ | carcinosarcoma | cartilage tumor |
| cementoma | myeloid sarcoma | chondroma | chordoma |
| choriocarcinoma | choroid plexus | clear-cell sarcoma of | craniopharyngioma |
| | papilloma | the kidney | |
| cutaneous T-cell | cervical cancer | colorectal cancer | Degos disease |
| lymphoma | | | |
| desmoplastic small | diffuse large B-cell | dysembryoplastic | dysgerminoma |
| round cell tumor | lymphoma | neuroepithelial tumor | |
| embryonal carcinoma | endocrine gland | endodermal sinus | enteropathy- |
| | neoplasm | tumor | associated T-cell |
| | | | lymphoma |
| esophageal cancer | fetus in fetu | fibroma | fibrosarcoma |
| follicular lymphoma | follicular thyroid | ganglioneuroma | gastrointestinal |
| | cancer | | cancer |
| germ cell tumor | gestational | giant cell | giant cell tumor of |
| | choriocarcinoma | fibroblastoma | the bone |
| glial tumor | glioblastoma | glioma | gliomatosis cerebri |
| | multiforme | | |
| glucagonoma | gonadoblastoma | granulosa cell tumor | gynandroblastoma |
| gallbladder cancer | gastric cancer | hairy cell leukemia | hemangioblastoma |
| head and neck cancer | hemangiopericytoma | hematological cancer | hepatoblastoma |
| hepatosplenic T-cell | Hodgkin's lymphoma | non-Hodgkin's | invasive lobular |
| lymphoma | | lymphoma | carcinoma |
| intestinal cancer | kidney cancer | laryngeal cancer | lentigo maligna |
| lethal midline | leukemia | leydig cell tumor | liposarcoma |
| carcinoma | | | |
| lung cancer | lymphangioma | lymphangiosarcoma | lymphoepithelioma |
| lymphoma | acute lymphocytic | acute myelogeous | chronic lymphocytic |
| | leukemia | leukemia | leukemia |
| liver cancer | small cell lung cancer | non-small cell lung | MALT lymphoma |
| | | cancer | |
| malignant fibrous | malignant peripheral | malignant triton | mantle cell |
| histiocytoma | nerve sheath tumor | tumor | lymphoma |
| marginal zone B-cell | mast cell leukemia | mediastinal germ cell | medullary carcinoma |
| lymphoma | | tumor | of the breast |
| medullary thyroid | medulloblastoma | melanoma | meningioma |
| cancer | | | |
| merkel cell cancer | mesothelioma | metastatic urothelial | mixed Mullerian |
| | | carcinoma | tumor |
| mucinous tumor | multiple myeloma | muscle tissue | mycosis fungoides |
| | | neoplasm | |
| myxoid liposarcoma | myxoma | myxosarcoma | nasopharyngeal |
| | | | carcinoma |
| neurinoma | neuroblastoma | neurofibroma | neuroma |
| nodular melanoma | ocular cancer | oligoastrocytoma | oligodendroglioma |
| oncocytoma | optic nerve sheath | optic nerve tumor | oral cancer |
| | meningioma | | |
| osteosarcoma | ovarian cancer | Pancoast tumor | papillary thyroid |
| | | | cancer |
| paraganglioma | pinealoblastoma | pineocytoma | pituicytoma |
| pituitary adenoma | pituitary tumor | plasmacytoma | polyembryoma |
| precursor T- | primary central | primary effusion | preimary peritoneal |
| lymphoblastic | nervous system | lymphoma | cancer |
| lymphoma | lymphoma | | |
| prostate cancer | pancreatic cancer | pharyngeal cancer | pseudomyxoma |
| | | | periotonei |
| renal cell carcinoma | renal medullary | retinoblastoma | rhabdomyoma |
| | carcinoma | | |
| rhabdomyosarcoma | Richter's | rectal cancer | sarcoma |
| | transformation | | |
| Schwannomatosis | seminoma | Sertoli cell tumor | sex cord-gonadal |
| | | | stromal tumor |
| signet ring cell | skin cancer | small blue round cell | small cell carcinoma |
| carcinoma | | tumors | |
| soft tissue sarcoma | somatostatinoma | soot wart | spinal tumor |
| splenic marginal zone | squamous cell | synovial sarcoma | Sezary's disease |
| lymphoma | carcinoma | | |
| small intestine cancer | squamous carcinoma | stomach cancer | T-cell lymphoma |
| testicular cancer | thecoma | thyroid cancer | transitional cell |
| | | | carcinoma |
| throat cancer | urachal cancer | urogenital cancer | urothelial carcinoma |
| uveal melanoma | uterine cancer | verrucous carcinoma | visual pathway |
| | | | glioma |
| vulvar cancer | vaginal cancer | Waldenstrom's | Warthin's tumor |
| | | macroglobulinemia | |
| Wilms' tumor | colon cancer | pancreatic ductal | |
| | | cancer | |

In another embodiment, the cancer is a solid tumor. In another embodiment, the cancer a hematological cancer. Exemplary hematological cancers include, but are not limited to, the cancers listed in Table 2. In another embodiment, the hematological cancer is acute lymphocytic leukemia, chronic lymphocytic leukemia (including B-cell chronic lymphocytic leukemia), or acute myeloid leukemia.

TABLE 2

| | |
|---|---|
| acute lymphocytic leukemia (ALL) | acute eosinophilic leukemia |
| acute myeloid leukemia (AML) | acute erythroid leukemia |
| chronic lymphocytic leukemia (CLL) | acute lymphoblastic leukemia |
| small lymphocytic lymphoma (SLL) | acute megakaryoblastic leukemia |
| multiple myeloma (MM) | acute monocytic leukemia |
| Hodgkins lymphoma (HL) | acute promyelocytic leukemia |
| non-Hodgkin's lymphoma (NHL) | acute myelogeous leukemia |
| mantle cell lymphoma (MCL) | B-cell prolymphocytic leukemia |
| marginal zone B-cell lymphoma | B-cell lymphoma |
| splenic marginal zone lymphoma | MALT lymphoma |
| follicular lymphoma (FL) | precursor T-lymphoblastic |
| Waldenstrom's | lymphoma |
| macroglobulinemia (WM) | T-cell lymphoma |
| diffuse large B-cell lymphoma | mast cell leukemia |
| (DLBCL) | adult T cell leukemia/lymphoma |
| marginal zone lymphoma (MZL) | aggressive NK-cell leukemia |
| hairy cell leukemia (HCL) | angioimmunoblastic T-cell |
| Burkitt's lymphoma (BL) | lymphoma |
| Richter's transformation | mantle cell lymphoma |

In another embodiment, the cancer is a leukemia, for example a leukemia selected from acute monocytic leukemia, acute myelogenous leukemia, chronic myelogenous leukemia, chronic lymphocytic leukemia and mixed lineage leukemia (MLL). In another embodiment the cancer is NUT-midline carcinoma. In another embodiment the cancer is multiple myeloma. In another embodiment the cancer is a lung cancer such as small cell lung cancer (SCLC). In another embodiment the cancer is a neuroblastoma. In another embodiment the cancer is Burkitt's lymphoma. In another embodiment the cancer is cervical cancer. In another embodiment the cancer is esophageal cancer. In another embodiment the cancer is ovarian cancer. In another embodiment the cancer is colorectal cancer. In another embodiment, the cancer is prostate cancer. In another embodiment, the cancer is breast cancer.

In another embodiment, the cancer is selected from the group consisting of acute monocytic leukemia, acute myelogenous leukemia, chronic myelogenous leukemia, chronic lymphocytic leukemia mixed lineage leukemia, NUT-midline carcinoma, multiple myeloma, small cell lung cancer, non-small cell lung cancer, neuroblastoma, Burkitt's lymphoma, cervical cancer, esophageal cancer, ovarian cancer, colorectal cancer, prostate cancer, breast cancer, bladder cancer, ovary cancer, glioma, sarcoma, esophageal squamous cell carcinoma, and papillary thyroid carcinoma.

In another embodiment, islatravir is administered to a subject in need thereof to treat breast, colon, lung, pancreatic ductal, prostate, ovarian, or head and neck cancer. In another embodiment, the cancer is breast cancer. In another embodiment, the cancer is colon cancer. In another embodiment, the cancer is lung cancer, e.g., small cell lung cancer or non-small cell lung cancer. In another embodiment, the cancer is pancreatic ductal cancer. In another embodiment, the cancer is prostate cancer. In another embodiment, the cancer is ovarian cancer. In another embodiment, the cancer is head and neck cancer.

In some embodiments, the patient is also administered at least one second therapeutic agent useful for the treatment of cancer. In some embodiments, the second therapeutic agent is an epigenetic drug. As used herein, the term “epigenetic drug” refers to a therapeutic agent that targets an epigenetic regulator. Examples of epigenetic regulators include the histone lysine methyltransferases, histone arginine methyl transferases, histone demethylases, histone deacetylases, histone acetylases, and DNA methyltransferases. Histone deacetylase inhibitors include, but are not limited to, vorinostat.

In another embodiment, chemotherapeutic agents or other anti-proliferative agents can be combined with islatravir to treat proliferative diseases and cancer. Examples of therapies and anticancer agents that can be used in combination with islatravir include surgery, radiotherapy (e.g., gamma-radiation, neutron beam radiotherapy, electron beam radiotherapy, proton therapy, brachytherapy, and systemic radioactive isotopes), endocrine therapy, a biologic response modifier (e.g., an interferon, an interleukin, tumor necrosis factor (TNF), hyperthermia and cryotherapy, an agent to attenuate any adverse effect (e.g., an antiemetic), and any other approved chemotherapeutic drug.

Examples of antiproliferative compounds include, but are not limited to, an aromatase inhibitor; an anti-estrogen; an anti-androgen; a gonadorelin agonist; a topoisomerase I inhibitor; a topoisomerase II inhibitor; a microtubule active agent; an alkylating agent; a retinoid, a carontenoid, or a tocopherol; a cyclooxygenase inhibitor; an MMP inhibitor; an mTOR inhibitor; an antimetabolite; a platin compound; a methionine aminopeptidase inhibitor; a bisphosphonate; an antiproliferative antibody; a heparanase inhibitor; an inhibitor of Ras oncogenic isoforms; a telomerase inhibitor; a proteasome inhibitor; a compound used in the treatment of hematologic malignancies; a Flt-3 inhibitor; an Hsp90 inhibitor; a kinesin spindle protein inhibitor; a MEK inhibitor; an antitumor antibiotic; a nitrosourea; a compound targeting/decreasing protein or lipid kinase activity, a compound targeting/decreasing protein or lipid phosphatase activity, or any further anti-angiogenic compound.

Nonlimiting exemplary aromatase inhibitors include, but are not limited to, steroids, such as atamestane, exemestane, and formestane, and non-steroids, such as aminoglutethimide, roglethimide, pyridoglutethimide, trilostane, testolactone, ketokonazole, vorozole, fadrozole, anastrozole, and letrozole.

Nonlimiting anti-estrogens include, but are not limited to, tamoxifen, fulvestrant, raloxifene, and raloxifene hydrochloride. Anti-androgens include, but are not limited to, bicalutamide. Gonadorelin agonists include, but are not limited to, abarelix, goserelin, and goserelin acetate.

Exemplary topoisomerase I inhibitors include, but are not limited to, topotecan, gimatecan, irinotecan, camptothecin and its analogues, 9-nitrocamptothecin, and the macromolecular camptothecin conjugate PNU-166148. Topoisomerase II inhibitors include, but are not limited to, anthracyclines, such as doxorubicin, daunorubicin, epirubicin, idarubicin, and nemorubicin; anthraquinones, such as mitoxantrone and losoxantrone; and podophillotoxines, such as etoposide and teniposide.

Microtubule active agents include microtubule stabilizing, microtubule destabilizing compounds, and microtubulin polymerization inhibitors including, but not limited to, taxanes, such as paclitaxel and docetaxel; vinca alkaloids, such as vinblastine, vinblastine sulfate, vincristine, and vincristine sulfate, and vinorelbine; discodermolides; cochicine and epothilones and derivatives thereof.

Exemplary nonlimiting alkylating agents include cyclophosphamide, ifosfamide, melphalan, and nitrosoureas, such as carmustine and lomustine.

Exemplary nonlimiting cyclooxygenase inhibitors include Cox-2 inhibitors, 5-alkyl substituted 2-arylaminophenylacetic acid and derivatives, such as celecoxib, rofecoxib, etoricoxib, valdecoxib, or a 5-alkyl-2-arylaminophenylacetic acid, such as lumiracoxib.

Exemplary nonlimiting matrix metalloproteinase inhibitors (“MMP inhibitors”) include collagen peptidomimetic and nonpeptidomimetic inhibitors, tetracycline derivatives, batimastat, marimastat, prinomastat, metastat, BMS-279251, BAY 12-9566, TAA211, MMI270B, and AAJ996.

Exemplary nonlimiting mTOR inhibitors include compounds that inhibit the mammalian target of rapamycin (mTOR) and possess antiproliferative activity such as sirolimus, everolimus, CCI-779, and ABT578.

Exemplary nonlimiting antimetabolites include 5-fluorouracil (5-FU), capecitabine, gemcitabine, DNA demethylating compounds, such as 5-azacytidine and decitabine, methotrexate and edatrexate, and folic acid antagonists, such as pemetrexed.

Exemplary nonlimiting platin compounds include carboplatin, cis-platin, cisplatinum, and oxaliplatin.

Exemplary nonlimiting methionine aminopeptidase inhibitors include bengamide or a derivative thereof and PPI-2458.

Exemplary nonlimiting bisphosphonates include etridonic acid, clodronic acid, tiludronic acid, pamidronic acid, alendronic acid, ibandronic acid, risedronic acid, and zoledronic acid.

Exemplary nonlimiting antiproliferative antibodies include trastuzumab, trastuzumab-DMI, cetuximab, bevacizumab, rituximab, PR064553, and 2C4. The term “antibody” is meant to include intact monoclonal antibodies, polyclonal antibodies, multispecific antibodies formed from at least two intact antibodies, and antibody fragments, so long as they exhibit the desired biological activity.

Exemplary nonlimiting heparanase inhibitors include compounds that target, decrease, or inhibit heparin sulfate degradation, such as PI-88 and OGT2115.

The term “an inhibitor of Ras oncogenic isoforms,” such as H-Ras, K-Ras, or N-Ras, as used herein refers to a compound which targets, decreases, or inhibits the oncogenic activity of Ras, for example, a farnesyl transferase inhibitor, such as L-744832, DK8G557, tipifarnib, and lonafarnib.

Exemplary nonlimiting telomerase inhibitors include compounds that target, decrease, or inhibit the activity of telomerase, such as compounds that inhibit the telomerase receptor, such as telomestatin.

Exemplary nonlimiting proteasome inhibitors include compounds that target, decrease, or inhibit the activity of the proteasome including, but not limited to, bortezomid.

The phrase “compounds used in the treatment of hematologic malignancies” as used herein includes FMS-like tyrosine kinase inhibitors, which are compounds targeting, decreasing or inhibiting the activity of FMS-like tyrosine kinase receptors (Flt-3R); interferon, I-β-D-arabinofuransylcytosine (ara-c), and bisulfan; and ALK inhibitors, which are compounds which target, decrease, or inhibit anaplastic lymphoma kinase.

Exemplary nonlimiting Flt-3 inhibitors include PKC412, midostaurin, a staurosporine derivative, SU11248, and MLN518.

Exemplary nonlimiting HSP90 inhibitors include compounds targeting, decreasing, or inhibiting the intrinsic ATPase activity of HSP90; or degrading, targeting, decreasing or inhibiting the HSP90 client proteins via the ubiquitin proteosome pathway. Compounds targeting, decreasing or inhibiting the intrinsic ATPase activity of HSP90 are especially compounds, proteins, or antibodies that inhibit the ATPase activity of HSP90, such as 17-allylamino, 17-demethoxygeldanamycin (17AAG), a geldanamycin derivative; other geldanamycin related compounds; radicicol and HDAC inhibitors.

The phrase “a compound targeting/decreasing a protein or lipid kinase activity; or a protein or lipid phosphatase activity; or any further anti-angiogenic compound” as used herein includes a protein tyrosine kinase and/or serine and/or threonine kinase inhibitor or lipid kinase inhibitor, such as a) a compound targeting, decreasing, or inhibiting the activity of the platelet-derived growth factor-receptors (PDGFR), such as a compound that targets, decreases, or inhibits the activity of PDGFR, such as an N-phenyl-2-pyrimidine-amine derivatives, such as imatinib, SUlOl, SU6668, and GFB-111; b) a compound targeting, decreasing, or inhibiting the activity of the fibroblast growth factor-receptors (FGFR); c) a compound targeting, decreasing, or inhibiting the activity of the insulin-like growth factor receptor I (IGF-IR), such as a compound that targets, decreases, or inhibits the activity of IGF-IR; d) a compound targeting, decreasing, or inhibiting the activity of the Trk receptor tyrosine kinase family, or ephrin B4 inhibitors; e) a compound targeting, decreasing, or inhibiting the activity of the Axl receptor tyrosine kinase family; f) a compound targeting, decreasing, or inhibiting the activity of the Ret receptor tyrosine kinase; g) a compound targeting, decreasing, or inhibiting the activity of the Kit/SCFR receptor tyrosine kinase, such as imatinib; h) a compound targeting, decreasing, or inhibiting the activity of the c-Kit receptor tyrosine kinases, such as imatinib; i) a compound targeting, decreasing, or inhibiting the activity of members of the c-Abl family, their gene-fusion products (e.g. Bcr-Abl kinase) and mutants, such as an N-phenyl-2-pyrimidine-amine derivative, such as imatinib or nilotinib; PD180970; AG957; NSC 680410; PD173955; or dasatinib; j) a compound targeting, decreasing, or inhibiting the activity of members of the protein kinase C (PKC) and Raf family of serine/threonine kinases, members of the MEK, SRC, JAK, FAK, PDK1, PKB/Akt, and Ras/MAPK family members, and/or members of the cyclin-dependent kinase family (CDK), such as a staurosporine derivative disclosed in U.S. Pat. No. 5,093,330, such as midostaurin; examples of further compounds include UCN-01, safingol, BAY 43-9006, bryostatin 1, perifosine; ilmofosine; RO 318220 and RO 320432; GO 6976; Isis 3521; LY333531/LY379196; a isochinoline compound; a farnesyl transferase inhibitor; PD184352 or QAN697, or AT7519; k) a compound targeting, decreasing or inhibiting the activity of a protein-tyrosine kinase, such as imatinib mesylate or a tyrphostin, such as Tyrphostin A23/RG-50810; AG 99; Tyrphostin AG 213; Tyrphostin AG 1748; Tyrphostin AG 490; Tyrphostin B44; Tyrphostin B44 (+) enantiomer; Tyrphostin AG 555; AG 494; Tyrphostin AG 556, AG957 and adaphostin (4-{[(2,5-dihydroxyphenyl)methyl]amino}-benzoic acid adamantyl ester; NSC 680410, adaphostin); 1) a compound targeting, decreasing, or inhibiting the activity of the epidermal growth factor family of receptor tyrosine kinases (EGFR, ErbB2, ErbB3, ErbB4 as homo- or heterodimers) and their mutants, such as CP 358774, ZD 1839, ZM 105180; trastuzumab, cetuximab, gefitinib, erlotinib, OSI-774, C1-1033, EKB-569, GW-2016, antibodies EL1, E2.4, E2.5, E6.2, E6.4, E2.11, E6.3 and E7.6.3, and 7H-pyrrolo-[2,3-d]pyrimidine derivatives; and m) a compound targeting, decreasing, or inhibiting the activity of the c-Met receptor.

Exemplary compounds that target, decrease, or inhibit the activity of a protein or lipid phosphatase include inhibitors of phosphatase 1, phosphatase 2A, or CDC25, such as okadaic acid or a derivative thereof.

Further anti-angiogenic compounds include compounds having another mechanism for their activity unrelated to protein or lipid kinase inhibition, e.g., thalidomide and TNP-470.

Additional, nonlimiting, exemplary chemotherapeutic compounds, one or more of which may be used in combination with islatravir, include: daunorubicin, adriamycin, Ara-C, VP-16, teniposide, mitoxantrone, idarubicin, carboplatinum, PKC412, 6-mercaptopurine (6-MP), fludarabine phosphate, octreotide, SOM230, FTY720, 6-thioguanine, cladribine, 6-mercaptopurine, pentostatin, hydroxyurea, 2-hydroxy-1H-isoindole-1,3-dione derivatives, 1-(4-chloroanilino)-4-(4-pyridylmethyl)phthalazine or a pharmaceutically acceptable salt thereof, 1-(4-chloroanilino)-4-(4-pyridylmethyl)phthalazine succinate, angiostatin, endostatin, anthranilic acid amides, ZD4190, ZD6474, SU5416, SU6668, bevacizumab, rhuMAb, rhuFab, macugon; FLT-4 inhibitors, FLT-3 inhibitors, VEGFR-2 IgGI antibody, RPI 4610, bevacizumab, porfimer sodium, anecortave, triamcinolone, hydrocortisone, 11-a-epihydrocotisol, cortex olone, 17a-hydroxyprogesterone, corticosterone, desoxycorticosterone, testosterone, estrone, dexamethasone, fluocinolone, a plant alkaloid, a hormonal compound and/or antagonist, a biological response modifier, such as a lymphokine or interferon, an antisense oligonucleotide or oligonucleotide derivative, shRNA, and siRNA.

In another embodiment, the second therapeutically active agent is an immune checkpoint inhibitor. Examples of immune checkpoint inhibitors include PD-1 inhibitors, PD-L1 inhibitors, CTLA-4 inhibitors, LAG3 inhibitors, TIM3 inhibitors, cd47 inhibitors, and B7-H1 inhibitors. Thus, in one embodiment, islatravir is administered in combination with an immune checkpoint inhibitor is selected from the group consisting of a PD-1 inhibitor, a PD-L1 inhibitor, a CTLA-4 inhibitor, a LAG3 inhibitor, a TIM3 inhibitor, and a cd47 inhibitor.

In another embodiment, the immune checkpoint inhibitor is a programmed cell death (PD-1) inhibitor. PD-1 is a T-cell coinhibitory receptor that plays a pivotal role in the ability of tumor cells to evade the host's immune system. Blockage of interactions between PD-1 and PD-L1, a ligand of PD-1, enhances immune function and mediates antitumor activity. Examples of PD-1 inhibitors include antibodies that specifically bind to PD-1. Particular anti-PD-1 antibodies include, but are not limited to nivolumab, pembrolizumab, STI-A1014, and pidilzumab. For a general discussion of the availability, methods of production, mechanism of action, and clinical studies of anti-PD-1 antibodies, see U.S. 2013/0309250, U.S. Pat. Nos. 6,808,710, 7,595,048, 8,008,449, 8,728,474, 8,779,105, 8,952,136, 8,900,587, 9,073,994, 9,084,776, and Naido et al., British Journal of Cancer 111:2214-19 (2014).

In another embodiment, the immune checkpoint inhibitor is a PD-L1 (also known as B7-H1 or CD274) inhibitor. Examples of PD-L1 inhibitors include antibodies that specifically bind to PD-L1. Particular anti-PD-L1 antibodies include, but are not limited to, avelumab, atezolizumab, durvalumab, and BMS-936559. For a general discussion of the availability, methods of production, mechanism of action, and clinical studies, see U.S. Pat. No. 8,217,149, U.S. 2014/0341917, U.S. 2013/0071403, WO 2015036499, and Naido et al., British Journal of Cancer 111:2214-19 (2014).

In another embodiment, the immune checkpoint inhibitor is a CTLA-4 inhibitor. CTLA-4, also known as cytotoxic T-lymphocyte antigen 4, is a protein receptor that downregulates the immune system. CTLA-4 is characterized as a “brake” that binds costimulatory molecules on antigen-presenting cells, which prevents interaction with CD28 on T cells and also generates an overtly inhibitory signal that constrains T cell activation. Examples of CTLA-4 inhibitors include antibodies that specifically bind to CTLA-4. Particular anti-CTLA-4 antibodies include, but are not limited to, ipilimumab and tremelimumab. For a general discussion of the availability, methods of production, mechanism of action, and clinical studies, see U.S. Pat. Nos. 6,984,720, 6,207,156, and Naido et al., British Journal of Cancer 111:2214-19 (2014).

In another embodiment, the immune checkpoint inhibitor is a LAG3 inhibitor. LAG3, Lymphocyte Activation Gene 3, is a negative co-simulatory receptor that modulates T cell homeostatis, proliferation, and activation. In addition, LAG3 has been reported to participate in regulatory T cells (Tregs) suppressive function. A large proportion of LAG3 molecules are retained in the cell close to the microtubule-organizing center, and only induced following antigen specific T cell activation. U.S. 2014/0286935. Examples of LAG3 inhibitors include antibodies that specifically bind to LAG3. Particular anti-LAG3 antibodies include, but are not limited to, GSK2831781. For a general discussion of the availability, methods of production, mechanism of action, and studies, see, U.S. 2011/0150892, U.S. 2014/0093511, U.S. 20150259420, and Huang et al., Immunity 21:503-13 (2004).

In another embodiment, the immune checkpoint inhibitor is a TIM3 inhibitor. TIM3, T-cell immunoglobulin and mucin domain 3, is an immune checkpoint receptor that functions to limit the duration and magnitude of T_{H}1 and T_{C}1 T-cell responses. The TIM3 pathway is considered a target for anticancer immunotherapy due to its expression on dysfunctional CD8+ T cells and Tregs, which are two reported immune cell populations that constitute immunosuppression in tumor tissue. Anderson, Cancer Immunology Research 2:393-98 (2014). Examples of TIM3 inhibitors include antibodies that specifically bind to TIM3. For a general discussion of the availability, methods of production, mechanism of action, and studies of TIM3 inhibitors, see U.S. 20150225457, U.S. 20130022623, U.S. Pat. No. 8,522,156, Ngiow et al., Cancer Res 71: 6567-71 (2011), Ngiow, et al., Cancer Res 71:3540-51 (2011), and Anderson, Cancer Immunology Res 2:393-98 (2014).

In another embodiment, the immune checkpoint inhibitor is a cd47 inhibitor. See Unanue, E. R., PNAS 110:10886-87 (2013).

The term “antibody” is meant to include intact monoclonal antibodies, polyclonal antibodies, multispecific antibodies formed from at least two intact antibodies, and antibody fragments, so long as they exhibit the desired biological activity. In another embodiment, “antibody” is meant to include soluble receptors that do not possess the Fc portion of the antibody. In one embodiment, the antibodies are humanized monoclonal antibodies and fragments thereof made by means of recombinant genetic engineering.

Another class of immune checkpoint inhibitors include polypeptides that bind to and block PD-1 receptors on T-cells without triggering inhibitor signal transduction. Such peptides include B7-DC polypeptides, B7-H1 polypeptides, B7-1 polypeptides and B7-2 polypeptides, and soluble fragments thereof, as disclosed in U.S. Pat. No. 8,114,845.

Another class of immune checkpoint inhibitors include compounds with peptide moieties that inhibit PD-1 signaling. Examples of such compounds are disclosed in U.S. Pat. No. 8,907,053.

Another class of immune checkpoint inhibitors include inhibitors of certain metabolic enzymes, such as indoleamine 2,3-dioxygenase (IDO), which is expressed by infiltrating myeloid cells and tumor cells. The IDO enzyme inhibits immune responses by depleting amino acids that are necessary for anabolic functions in T cells or through the synthesis of particular natural ligands for cytosolic receptors that are able to alter lymphocyte functions. Pardoll, Nature Reviews. Cancer 12:252-64 (2012); Lob, Cancer Immunol Immunother 58:153-57 (2009). Particular IDO blocking agents include, but are not limited to levo-1-methyl typtophan (L-1MT) and 1-methyl-tryptophan (1MT). Qian et al., Cancer Res 69:5498-504 (2009); and Lob et al., Cancer Immunol Immunother 58:153-7 (2009).

In one embodiment, the immune checkpoint inhibitor is nivolumab, pembrolizumab, pidilizumab, STI-A1110, avelumab, atezolizumab, durvalumab, STI-A1014, ipilimumab, tremelimumab, GSK2831781, BMS-936559 or MED14736.

When the RTI is an FDA approved drug, the RTI may be administered in therapeutically effective amounts that are approved for therapeutic use. In other embodiments, the amounts effective can be determined with no more than routine experimentation. For example, amounts effective may range from about 1 ng/kg to about 200 mg/kg, about 1 μg/kg to about 100 mg/kg, or about 1 mg/kg to about 50 mg/kg. The dosage of a composition can be at any dosage including, but not limited to, about 1 μg/kg. The dosage of a composition may be at any dosage including, but not limited to, about 1 μg/kg, about 10 μg/kg, about 25 μg/kg, about 50 μg/kg, about 75 μg/kg, about 100 μg/kg, about 125 μg/kg, about 150 μg/kg, about 175 μg/kg, about 200 μg/kg, about 225 ag/kg, about 250 μg/kg, about 275 μg/kg, about 300 μg/kg, about 325 μg/kg, about 350 ag/kg, about 375 μg/kg, about 400 μg/kg, about 425 μg/kg, about 450 μg/kg, about 475 ag/kg, about 500 μg/kg, about 525 μg/kg, about 550 μg/kg, about 575 μg/kg, about 600 μg/kg, about 625 μg/kg, about 650 μg/kg, about 675 μg/kg, about 700 μg/kg, about 725 μg/kg, about 750 μg/kg, about 775 μg/kg, about 800 μg/kg, about 825 μg/kg, about 850 μg/kg, about 875 μg/kg, about 900 μg/kg, about 925 μg/kg, about 950 μg/kg, about 975 μg/kg, about 1 mg/kg, about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 60 mg/kg, about 70 mg/kg, about 80 mg/kg, about 90 mg/kg, about 100 mg/kg, about 125 mg/kg, about 150 mg/kg, about 175 mg/kg, about 200 mg/kg, or more. In other embodiments, the dosage is 1 mg-500 mg. In some embodiments, the dosage is 10, 20, 30, 40, 50, 60, 70, 80, 90, 100, 110, 120, 130, 140, or 150 mg. These doses may be unitary or divided and may be administered one or more times per day. The above dosages are exemplary of the average case, but there can be individual instances in which higher or lower dosages are merited, and such are within the scope of this disclosure. In practice, the physician determines therapeutically effective amounts and the actual dosing regimen that is most suitable for an individual subject, which can vary with the age, weight, and response of the particular subject.

The RTI may be administered once, twice or three times per day for 1 day to the end of life, or for 1 day to 1, 2, 3, 4, 5, 6, 7, 8, 9, or 10 or more years, or until the RTI causes unacceptable side effects or is no longer useful.

In some embodiments, when the method is a method for the treatment of breast cancer, the at least one second therapeutic agent is Soltamox® (tamoxifen), Arimidex® (anastrozole), Femara® (letrozole), Aromasin® (exemestane), Herceptin® (trastuzumab), Abraxane® (paclitaxel), Cytoxan® (cyclophosphamide), Taxol® (paclitaxel), Afinitor® (everolimus), Taxotere® (docetaxel), Xeloda® (capecitabine), Trexall® (methotrexate), Faslodex (fulvestrant), Adriamycin® (doxorubicin), Perjeta® (pertuzumab), Gemzar (gemcitabine), Tykerb® (lapatinib), Adrucil® (fluorouracil), Ibrance® (palbociclib), Verzenio® (abemaciclib), Fareston® (toremifene), Halaven® (eribulin), Menest, Kadcyla® (ado-trastuzumab emtransine), Androxy® (fluoxymesterone), Avastin® (bevacizumab), esterified estrogens, Herzuma® (trastuzumab), Ixempra® (ixabepilone), Kanjinti® (trastuzumab), Kisqali® (ribociclib), Ogivri® (trastuzumab), Ontruzant® (trastuzumab), Tepadina® (thiotepa), Trazimera® (trastuzumab), Velban® (vinblastine), Piqray® (alpelisib), Tecentriq® (atezolizumab), Enhertu® (fam-trastuzumab deruxtecan), Herceptin, Hylecta™ (hyaluronidase/trastuzumab), Infugem® (gemcitabine), Kisqali® Femara® Co-Pack (ribociclib and letrozole), Talzenna® (talazoparib), Trodelvy® (sacituzumab) or Tukysa™ (tukatinib).

In some embodiments, when the method is a method for the treatment of colon cancer, the at least one second therapeutic agent is Xeloda® (capecitabine), Eloxatin® (oxaliplatin), fluorouracil, Avastin® (bevacizumab), leucovorin, Camptosar® (irinotecan), Stivarga® (regorafenib), Erbitux® (cetuximab), Vectibix® (panitumumab), Lonsurf® (tipiracil/trifluridine), Zaltrap® (ziv-aflibercept), Betaseron® (interferon beta-1b), Fusilev® (levoleucovorin), Wellcocorin® (methotrexate), Keytruda® (pembrolizumab), Mvasi® (bevacizumab-awwb), Cyramza® (ramucirumab), Yervoy® (ipilmumab), Opdivo® (nivolumab), Braftovi® (encorafenib), Khapzory® (levoleucovorin) or Zirabev® (bevacizumab-bvzr).

In some embodiments, when the method is a method for the treatment of lung cancer, the at least one second therapeutic agent is Etopophos® (etoposide), Hycamtin® (topotecan), VePesid® (etoposide), Toposar® (etoposide), Opdivo® (nivolumab), Keytruda® (pembrolizumab), Tecentriq® (atezolizumab), Imfinizi® (durvalumab), methotrexate, cyclophosphamide, Carboplatin, Cisplatin, docetaxel, Gemcitabine, Irinotecan, Paclitaxel, Pemetrexed, Vinblastine, or Vinorelbine.

In some embodiments, when the method is a method for the treatment of pancreatic ductal cancer, the at least one second therapeutic agent is Gemzar® (Gemcitabine), fluorouracil, Afinitor® (everolimus), Tarceva® (erlotinib), Abraxane® (paclitaxel), capecitabine, Sutent® (sunitinib), pancreatin, methotrexate, Zanosar® (streptozocin), Mutamycin® (mitomycin), Onivyde® (irinotecan), bevacizumab, cetuximab, Infugem® (gemcitabine) or Lynparza® (olaparib).

In some embodiments, when the method is a method for the treatment of head and neck cancer, the at least one second therapeutic agent is Erbituz® (cetuximab), Taxotere® (docetaxel), Trexall® (methotrexate), Keytruda® (pembrolizumab) or Opdivo® (nivolumab).

In some embodiments, when the method is a method for the treatment of prostate cancer the at least one second therapeutic agent is Suprefact® (buserelin), Firmagon® (degarelix), Zoladex® (goserelin), Vantas® (histrelin), Eligard® (leuprolide), Orgovyx® (relugolix), Trelstar® (triptorelin), Casodex® (bicalutamide), Eulexin® (flutamide), Nilandron® (nilutamide), Zytiga® (biraterone acetate), Erleada® (apalutamide), or Xtandi® (enzalutamide).

In some embodiments, the at least one second therapeutic agent is a STING agonist. Exemplary STING agonists include E7766, MIW815, SNX281, and TAK-676. See, e.g., Aval et al., Journal of Clinical Medicine 9:3323 (2020); Su et al., Theranostics 9:7759-7771 (2019).

The RTI and at least one second therapeutic agent may be administered separately or together as part of a unitary pharmaceutical composition.

In some embodiments, the patient is (a) not infected with the HIV virus, (b) not suspected of being infected with the HIV virus, (c) not being treated for the HIV virus, and/or (d) not being treated to prevent the HIV virus.

The terms “patient” and “subject” as used herein are synonymous terms referring to any human or animal that is in need of or might benefit from administration of a RTI for treating cancer. Foremost among such subjects are mammals, e.g., humans, although the methods and compositions provided herein are not intended to be so limited. Other subjects include veterinary animals, e.g., cows, sheep, pigs, horses, dogs, cats and the like. In one embodiment, the subject is a human. In one embodiment, the subject is an animal.

### Salts, Pharmaceutical Compositions, and Kits

The methods of the present disclosure can be accomplished by administering RTI as the neat compound or as a pharmaceutical composition. Administration of a pharmaceutical composition, or a neat RTI can be performed before, during, or after the clinical diagnosis of the cancer. Typically, the pharmaceutical compositions are sterile, and contain no toxic, carcinogenic, or mutagenic compounds that would cause an adverse reaction when administered.

Further provided are kits comprising the RTI and, optionally, at least one second therapeutic agent useful for the treatment of cancer associated, packaged separately or together, and an insert having instructions for using these active agents. In one embodiment, the RTI is packaged alone together with instructions to administered together with the at least one second therapeutic agent. The RTI and the at least one second therapeutic agent can be administered simultaneously or sequentially to achieve the desired effect. In addition, the RTI and the at least one second therapeutic agent can be administered from a single composition or two separate compositions. The second therapeutic agent is administered in an amount to provide its desired therapeutic effect. The effective dosage range for each optional therapeutic agent is known in the art, and the optional therapeutic agent is administered to an individual in need thereof within such established ranges.

The present disclosure encompasses the preparation and use of salts of a RTI. As used herein, a “pharmaceutically acceptable salt” refers to salts or zwitterionic forms of a RTI. Salts of a RTI can be prepared during the final isolation and purification of the compound or separately by reacting the compound with a suitable acid. The pharmaceutically acceptable salts of a RTI can be acid addition salts formed with pharmaceutically acceptable acids. Examples of acids which can be employed to form pharmaceutically acceptable salts include inorganic acids such as nitric, boric, hydrochloric, hydrobromic, sulfuric, and phosphoric, and organic acids such as oxalic, maleic, succinic, and citric. Non-limiting examples of salts of a RTI include, but are not limited to, the hydrochloride, hydrobromide, hydroiodide, sulfate, bisulfate, 2-hydroxyethansulfonate, phosphate, hydrogen phosphate, acetate, adipate, alginate, aspartate, benzoate, bisulfate, butyrate, camphorate, camphorsulfonate, digluconate, glycerolphsphate, hemisulfate, heptanoate, hexanoate, formate, succinate, fumarate, maleate, ascorbate, isethionate, salicylate, methanesulfonate, mesitylenesulfonate, naphthylenesulfonate, nicotinate, 2-naphthalenesulfonate, oxalate, pamoate, pectinate, persulfate, 3-phenylproprionate, picrate, pivalate, propionate, trichloroacetate, trifluoroacetate, phosphate, glutamate, bicarbonate, para-toluenesulfonate, undecanoate, lactate, citrate, tartrate, gluconate, methanesulfonate, ethanedisulfonate, benzene sulfonate, and p-toluenesulfonate salts.

The present disclosure encompasses the preparation and use of solvates of a RTI. Solvates typically do not significantly alter the physiological activity or toxicity of the compounds, and as such may function as pharmacological equivalents. The term “solvate” as used herein is a combination, physical association and/or solvation of a compound with a solvent molecule such as, e.g. a disolvate, monosolvate or hemisolvate, where the ratio of solvent molecule to compound is about 2:1, about 1:1 or about 1:2, respectively. This physical association involves varying degrees of ionic and covalent bonding, including hydrogen bonding. In certain instances, the solvate can be isolated, such as when one or more solvent molecules are incorporated into the crystal lattice of a crystalline solid. Thus, “solvate” encompasses both solution-phase and isolatable solvates. A RTI can be present as solvated forms with a pharmaceutically acceptable solvent, such as water, methanol, and ethanol. It is intended that the disclosure includes both solvated and unsolvated forms of a RTI. One type of solvate is a hydrate. A “hydrate” relates to a particular subgroup of solvates where the solvent molecule is water. Solvates typically can function as pharmacological equivalents. Preparation of solvates is known in the art. See, for example, M. Caira et al, J. Pharmaceut. Sci., 93(3):601-611 (2004), which describes the preparation of solvates of fluconazole with ethyl acetate and with water. Similar preparation of solvates, hemisolvates, hydrates, and the like are described by E. C. van Tonder et al., AAPS Pharm. Sci. Tech., 5(1):Article 12 (2004), and A. L. Bingham et al., Chem. Commun. 603-604 (2001). A typical, non-limiting, process of preparing a solvate would involve dissolving a RTI in a desired solvent (organic, water, or a mixture thereof) at temperatures above 20° C. to about 25° C., then cooling the solution at a rate sufficient to form crystals, and isolating the crystals by known methods, e.g., filtration. Analytical techniques such as infrared spectroscopy can be used to confirm the presence of the solvate in a crystal of the solvate.

The RTI is typically are administered in admixture with a pharmaceutical carrier to give a pharmaceutical composition selected with regard to the intended route of administration and standard pharmaceutical practice. Pharmaceutical compositions for use in accordance with the present disclosure are formulated in a conventional manner using one or more physiologically acceptable carriers comprising excipients and/or auxiliaries that facilitate processing of the RTI. These pharmaceutical compositions can be manufactured, for example, by conventional mixing, dissolving, granulating, dragee-making, emulsifying, encapsulating, entrapping, or lyophilizing processes. Proper formulation is dependent upon the route of administration chosen. When a therapeutically effective amount of a RTI is administered orally, the composition typically is in the form of a tablet, capsule, powder, solution, or elixir. When administered in tablet form, the composition additionally can contain a solid carrier, such as a gelatin or an adjuvant. The tablet, capsule, and powder contain about 0.01% to about 95%, and preferably from about 1% to about 50%, of a RTI, or a pharmaceutically acceptable salt or solvate thereof, or a tautomer thereof. When administered in liquid form, a liquid carrier, such as water, petroleum, or oils of animal or plant origin, can be added. The liquid form of the composition can further contain physiological saline solution, dextrose or other saccharide solutions, or glycols. When administered in liquid form, the composition contains about 0.1% to about 90%, and preferably about 1% to about 50%, by weight, of a RTI.

When a therapeutically effective amount of a RTI is administered by intravenous, cutaneous, or subcutaneous injection, the composition is in the form of a pyrogen-free, parenterally acceptable aqueous solution. The preparation of such parenterally acceptable solutions, having due regard to pH, isotonicity, stability, and the like, is within the skill in the art. A preferred composition for intravenous, cutaneous, or subcutaneous injection typically contains, an isotonic vehicle.

A RTI can be readily combined with pharmaceutically acceptable carriers well-known in the art. Standard pharmaceutical carriers are described in Remington's Pharmaceutical Sciences, Mack Publishing Co., Easton, Pa., 19th ed. 1995. Such carriers enable the active agents to be formulated as tablets, pills, dragees, capsules, liquids, gels, syrups, slurries, suspensions and the like, for oral ingestion by a subject to be treated. Pharmaceutical preparations for oral use can be obtained by adding a RTI to a solid excipient, optionally grinding the resulting mixture, and processing the mixture of granules, after adding suitable auxiliaries, if desired, to obtain tablets or dragee cores. Suitable excipients include, for example, fillers and cellulose preparations. If desired, disintegrating agents can be added.

A RTI can be formulated for parenteral administration by injection, e.g., by bolus injection or continuous infusion. Formulations for injection can be presented in unit dosage form, e.g., in ampules or in multidose containers, with an added preservative. The compositions can take such forms as suspensions, solutions, or emulsions in oily or aqueous vehicles, and can contain formulatory agents such as suspending, stabilizing, and/or dispersing agents.

Pharmaceutical compositions for parenteral administration include aqueous solutions of the RTI in water-soluble form. Additionally, suspensions of a compound of a RTI can be prepared as appropriate oily injection suspensions. Suitable lipophilic solvents or vehicles include fatty oils or synthetic fatty acid esters. Aqueous injection suspensions can contain substances which increase the viscosity of the suspension. Optionally, the suspension also can contain suitable stabilizers or agents that increase the solubility of the compounds and allow for the preparation of highly concentrated solutions. Alternatively, a present composition can be in powder form for constitution with a suitable vehicle, e.g., sterile pyrogen-free water, before use.

In particular, a RTI can be administered orally in the form of tablets containing excipients, such as starch or lactose, or in capsules or ovules, either alone or in admixture with excipients, or in the form of elixirs or suspensions containing flavoring or coloring agents. Such liquid preparations can be prepared with pharmaceutically acceptable additives, such as suspending agents. A RTI also can be injected parenterally, for example, intravenously, intramuscularly, subcutaneously, or intracoronarily. For parenteral administration, a RTI typically used in the form of a sterile aqueous solution which can contain other substances, for example, salts or monosaccharides, such as mannitol or glucose, to make the solution isotonic with blood.

All patents, patent application, and publications cited herein are fully incorporated by reference herein.

It is to be appreciated that the Detailed Description section, and not the Summary and Abstract sections, is intended to be used to interpret the claims. The Summary and Abstract sections may set forth one or more but not all exemplary embodiments of the present invention as contemplated by the inventor(s), and thus, are not intended to limit the present invention and the appended claims in any way.

The foregoing description of the specific embodiments will so fully reveal the general nature of the invention that others can, by applying knowledge within the skill of the art, readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The disclosure also provides the following particular embodiments with respect to methods for treating cancer in a patient in need thereof.

Embodiment 1. A method for treating cancer in patient in need thereof, the method comprising administering a therapeutically effective amount of a reverse transcriptase inhibitor (RTI) to the patient, wherein the RTI is administered to the patient according to an intermittent dosing schedule.

Embodiment 2. A method for increasing the expression of ORFp2 in the cancer cells of a patient, the method comprising administering a therapeutically effective amount of a RTI to the patient, wherein the RTI is administered to the patient according to an intermittent dosing schedule.

Embodiment 3. The method of Embodiments 1 or 2, wherein the RTI is a nucleoside reverse transcriptase inhibitor (NRTI).

Embodiment 4. The method of Embodiments 1 or 2, wherein the RTI is a non-nucleoside reverse transcriptase inhibitor (NNRTI).

Embodiment 5. The method of Embodiments 1 or 2, wherein the RTI is a LINE-1 inhibitor.

Embodiment 6. The method of Embodiments 1 or 2, wherein the RTI is islatravir, elvucitabine, lamivudine (3TC), zidovudine (AZT), tenofovir, tenofovir disoproxil, tenofovir alafenamide, stavudine (d4T), zalcitabine (ddC), didanosine (ddI), emtricitabine (FTC), entecavir (ETV), 2′,3′-dideoxyguanosine (ddG), 2′,3′-dideoxyadenosine (ddA), 2′-fluoro-2′,3′-dideoxyarabinosyladenine (F-ddA), efavirenz (EFV), nevirapine (NVP), or abacavir (ABC), adefovir dipivoxil, or telbivudine.

Embodiment 7. The method of Embodiment 6, wherein the RTI is lamivudine, stavudine, emtricitabine, abacavir, tenofovir alafenamide, zidovudine, zalcitabine, didanosine, tenofovir disoproxil, adefovir dipivoxil, entecavir, or telbivudine.

Embodiment 8. The method of Embodiment 6, wherein the RTI is lamivudine.

Embodiment 9. The method of Embodiment 6, wherein the RTI is stavudine.

Embodiment 10. The method of Embodiment 6, wherein the RTI is emtricitabine.

Embodiment 11. The method of Embodiment 6, wherein the RTI is abacavir.

Embodiment 12. The method of Embodiment 6, wherein the RTI is tenofovir alafenamide.

Embodiment 13. The method of Embodiment 6, wherein the RTI is zidovudine.

Embodiment 14. The method of Embodiment 6, wherein the RTI is zalcitabine.

Embodiment 15. The method of Embodiment 6, wherein the RTI is didanosine.

Embodiment 16. The method of Embodiment 6, wherein the RTI is tenofovir disoproxil.

Embodiment 17. The method of Embodiment 6, wherein the RTI is adefovir dipivoxil.

Embodiment 18. The method of Embodiment 6, wherein the RTI is entecavir.

Embodiment 19. The method of Embodiment 6, wherein the RTI is telbivudin.

Embodiment 20. The method of any one of Embodiments 1-19, wherein the cancer is any one or more of the cancers of Table 1, e.g., breast cancer, colon cancer, lung cancer, pancreatic ductal cancer, prostate cancer, ovarian cancer, or head and neck cancer.

Embodiment 21. The method of Embodiment 20, wherein the cancer is breast cancer.

Embodiment 22. The method of Embodiment 20, wherein the cancer is colon cancer.

Embodiment 23. The method of Embodiment 20, wherein the cancer is lung cancer.

Embodiment 24. The method of Embodiment 20, wherein the cancer is pancreatic ductal cancer.

Embodiment 25. The method of Embodiment 20, wherein the cancer is prostate cancer.

Embodiment 26. The method of Embodiment 20, wherein the cancer is ovarian cancer.

Embodiment 27. The method of Embodiment 20, wherein the cancer is head and neck cancer.

Embodiment 28. The method of any one of Embodiments 1-27, further comprising administering a therapeutically effective amount of at least one second therapeutic agent useful for treating the cancer.

Embodiment 29. The method of Embodiment 28 for the treatment of breast cancer, wherein the at least one second therapeutic agent is Soltamox® (tamoxifen), Arimidex® (anastrozole), Femara® (letrozole), Aromasin® (exemestane), Herceptin® (trastuzumab), Abraxane® (paclitaxel), Cytoxan® (cyclophosphamide), Taxol® (paclitaxel), Afinitor® (everolimus), Taxotere® (docetaxel), Xeloda® (capecitabine), Trexall® (methotrexate), Faslodex (fulvestrant), Adriamycin® (doxorubicin), Perjeta® (pertuzumab), Gemzar (gemcitabine), Tykerb® (lapatinib), Adrucil® (fluorouracil), Ibrance® (palbociclib), Verzenio® (abemaciclib), Fareston® (toremifene), Halaven® (eribulin), Menest, Kadcyla® (ado-trastuzumab emtransine), Androxy® (fluoxymesterone), Avastin® (bevacizumab), esterified estrogens, Herzuma® (trastuzumab), Ixempra® (ixabepilone), Kanjinti® (trastuzumab), Kisqali® (ribociclib), Ogivri® (trastuzumab), Ontruzant® (trastuzumab), Tepadina® (thiotepa), Trazimera® (trastuzumab), Velban® (vinblastine), Piqray® (alpelisib), Tecentriq® (atezolizumab), Enhertu® (fam-trastuzumab deruxtecan), Herceptin, Hylecta™ (hyaluronidase/trastuzumab), Infugem® (gemcitabine), Kisqali® Femara® Co-Pack (ribociclib and letrozole), Talzenna® (talazoparib), Trodelvy® (sacituzumab) or Tukysa™ (tukatinib).

Embodiment 30. The method of Embodiment 28 for the treatment of colon cancer, wherein the at least one second therapeutic agent is Xeloda® (capecitabine), Eloxatin® (oxaliplatin), fluorouracil, Avastin® (bevacizumab), leucovorin, Camptosar® (irinotecan), Stivarga® (regorafenib), Erbitux® (cetuximab), Vectibix® (panitumumab), Lonsurf® (tipiracil/trifluridine), Zaltrap® (ziv-aflibercept), Betaseron® (interferon beta-1b), Fusilev® (levoleucovorin), Wellcocorin® (methotrexate), Keytruda® (pembrolizumab), Mvasi® (bevacizumab-awwb), Cyramza® (ramucirumab), Yervoy® (ipilmumab), Opdivo® (nivolumab), Braftovi® (encorafenib), Khapzory® (levoleucovorin) or Zirabev® (bevacizumab-bvzr).

Embodiment 31. The method of Embodiment 28 for the treatment of lung cancer, wherein the at least one second therapeutic agent is Etopophos® (etoposide), Hycamtin® (topotecan), VePesid® (etoposide), Toposar® (etoposide), Opdivo® (nivolumab), Keytruda® (pembrolizumab), Tecentriq® (atezolizumab), Imfinizi® (durvalumab), methotrexate, cyclophosphamide, Carboplatin, Cisplatin, docetaxel, Gemcitabine, Irinotecan, Paclitaxel, Pemetrexed, Vinblastine, or Vinorelbine.

Embodiment 32. The method of Embodiment 28 for the treatment of pancreatic ductal cancer, wherein the at least one second therapeutic agent is Gemzar® (Gemcitabine), fluorouracil, Afinitor® (everolimus), Tarceva® (erlotinib), Abraxane® (paclitaxel), capecitabine, Sutent® (sunitinib), pancreatin, methotrexate, Zanosar® (streptozocin), Mutamycin® (mitomycin), Onivyde® (irinotecan), bevacizumab, cetuximab, Infugem® (gemcitabine) or Lynparza® (olaparib).

Embodiment 33. The method of Embodiment 28 for the treatment of head and neck cancer, wherein the at least one second therapeutic agent is Erbituz® (cetuximab), Taxotere® (docetaxel), Trexall® (methotrexate), Keytruda® (pembrolizumab) or Opdivo® (nivolumab).

Embodiment 34. The method of Embodiment 28 for the treatment of prostate cancer, wherein the at least one second therapeutic agent is Suprefact® (buserelin), Firmagon® (degarelix), Zoladex® (goserelin), Vantas® (histrelin), Eligard® (leuprolide), Orgovyx® (relugolix), Trelstar® (triptorelin), Casodex® (bicalutamide), Eulexin® (flutamide), Nilandron® (nilutamide), Zytiga® (biraterone acetate), Erleada® (apalutamide), or Xtandi® (enzalutamide).

Embodiment 35. The method of Embodiment 28, wherein the at least one second therapeutic agent is a STING agonist.

Embodiment 36. The method of any one of Embodiments 1-35, wherein the patient is (a) not infected with the HIV virus, (b) not suspected of being infected with the HIV virus, (c) not being treated for the HIV virus, and/or (d) not being treated to prevent the HIV virus.

Embodiment 37. A kit for carrying out the method of any one Embodiments 1-35, the kit comprising (i) a RTI; and (ii) and instructions for administering the RTI to a patient having cancer according to an intermittent dosing schedule.

Embodiment 38. The kit of Embodiment 36 further comprising at least one second therapeutic agent.

The disclosure also provides the following particular embodiments with respect to RTIs (and compositions thereof) for use to treat cancer in a subject.

Embodiment 1. A RTI for use in treating cancer in patient in need thereof, wherein the RTI is to be administered according to an intermittent dosing schedule.

Embodiment 2. A RTI for use in increasing the expression of ORFp2 in the cancer cells of a patient, wherein the RTI is to be administered to the patient according to an intermittent dosing schedule.

Embodiment 3. The RTI for use of Embodiments 1 or 2, wherein the RTI is a nucleoside reverse transcriptase inhibitor (NRTI).

Embodiment 4. The RTI for use of Embodiments 1 or 2, wherein the RTI is a non-nucleoside reverse transcriptase inhibitor (NNRTI).

Embodiment 5. The RTI for use of Embodiments 1 or 2, wherein the RTI is a LINE-1 inhibitor.

Embodiment 6. The RTI for use of Embodiments 1 or 2, wherein the RTI is islatravirelvucitabine, lamivudine (3TC), zidovudine (AZT), tenofovir, tenofovir disoproxil, tenofovir alafenamide, stavudine (d4T), zalcitabine (ddC), didanosine (ddI), emtricitabine (FTC), entecavir (ETV), 2′,3′-dideoxyguanosine (ddG), 2′,3′-dideoxyadenosine (ddA), 2′-fluoro-2′,3′-dideoxyarabinosyladenine (F-ddA), efavirenz (EFV), nevirapine (NVP), or abacavir (ABC), adefovir dipivoxil, or telbivudine.

Embodiment 7. The RTI for use of Embodiment 6, wherein the RTI is lamivudine, stavudine, emtricitabine, abacavir, tenofovir alafenamide, zidovudine, zalcitabine, didanosine, tenofovir disoproxil, adefovir dipivoxil, entecavir, or telbivudine.

Embodiment 8. The RTI for use of Embodiment 6, wherein the RTI is lamivudine.

Embodiment 9. The RTI for use of Embodiment 6, wherein the RTI is stavudine.

Embodiment 10. The RTI for use of Embodiment 6, wherein the RTI is emtricitabine.

Embodiment 11. The RTI for use of Embodiment 6, wherein the RTI is abacavir.

Embodiment 12. The RTI for use of Embodiment 6, wherein the RTI is tenofovir alafenamide.

Embodiment 13. The RTI for use of Embodiment 6, wherein the RTI is zidovudine.

Embodiment 14. The RTI for use of Embodiment 6, wherein the RTI is zalcitabine.

Embodiment 15. The RTI for use of Embodiment 6, wherein the RTI is didanosine.

Embodiment 16. The RTI for use of Embodiment 6, wherein the RTI is tenofovir disoproxil.

Embodiment 17. The RTI for use of Embodiment 6, wherein the RTI is adefovir dipivoxil.

Embodiment 18. The RTI for use of Embodiment 6, wherein the RTI is entecavir.

Embodiment 19. The RTI for use of Embodiment 6, wherein the RTI is telbivudine.

Embodiment 20. The RTI for use of any one of Embodiments 1-19, wherein the cancer is any one or more of the cancers of Table 1, e.g., breast cancer, colon cancer, lung cancer, pancreatic ductal cancer, prostate cancer, ovarian cancer, or head and neck cancer.

Embodiment 21. The RTI for use of Embodiment 20, wherein the cancer is breast cancer.

Embodiment 22. The RTI for use of Embodiment 20, wherein the cancer is colon cancer.

Embodiment 23. The RTI for use of Embodiment 20, wherein the cancer is lung cancer.

Embodiment 24. The RTI for use of Embodiment 20, wherein the cancer is pancreatic ductal cancer.

Embodiment 25. The RTI for use of Embodiment 20, wherein the cancer is prostate cancer.

Embodiment 26. The RTI for use of Embodiment 20, wherein the cancer is ovarian cancer.

Embodiment 27. The RTI for use of Embodiment 20, wherein the cancer is head and neck cancer.

Embodiment 28. The RTI for use of any one of Embodiments 1-27, further comprising administering a therapeutically effective amount of at least one second therapeutic agent useful for treating the cancer.

Embodiment 29. The RTI for use of Embodiment 28 for the treatment of breast cancer, wherein the at least one second therapeutic agent is Soltamox® (tamoxifen), Arimidex® (anastrozole), Femara® (letrozole), Aromasin® (exemestane), Herceptin® (trastuzumab), Abraxane® (paclitaxel), Cytoxan® (cyclophosphamide), Taxol® (paclitaxel), Afinitor® (everolimus), Taxotere® (docetaxel), Xeloda® (capecitabine), Trexall® (methotrexate), Faslodex (fulvestrant), Adriamycin® (doxorubicin), Perjeta® (pertuzumab), Gemzar (gemcitabine), Tykerb® (lapatinib), Adrucil® (fluorouracil), Ibrance® (palbociclib), Verzenio® (abemaciclib), Fareston® (toremifene), Halaven® (eribulin), Menest, Kadcyla® (ado-trastuzumab emtransine), Androxy® (fluoxymesterone), Avastin® (bevacizumab), esterified estrogens, Herzuma® (trastuzumab), Ixempra® (ixabepilone), Kanjinti® (trastuzumab), Kisqali® (ribociclib), Ogivri® (trastuzumab), Ontruzant® (trastuzumab), Tepadina® (thiotepa), Trazimera® (trastuzumab), Velban® (vinblastine), Piqray® (alpelisib), Tecentriq® (atezolizumab), Enhertu® (fam-trastuzumab deruxtecan), Herceptin, Hylecta™ (hyaluronidase/trastuzumab), Infugem® (gemcitabine), Kisqali® Femara® Co-Pack (ribociclib and letrozole), Talzenna® (talazoparib), Trodelvy® (sacituzumab) or Tukysa™ (tukatinib).

Embodiment 30. The RTI for use of Embodiment 28 for the treatment of colon cancer, wherein the at least one second therapeutic agent is Xeloda® (capecitabine), Eloxatin® (oxaliplatin), fluorouracil, Avastin® (bevacizumab), leucovorin, Camptosar® (irinotecan), Stivarga® (regorafenib), Erbitux® (cetuximab), Vectibix® (panitumumab), Lonsurf® (tipiracil/trifluridine), Zaltrap® (ziv-aflibercept), Betaseron® (interferon beta-1b), Fusilev® (levoleucovorin), Wellcocorin® (methotrexate), Keytruda® (pembrolizumab), Mvasi® (bevacizumab-awwb), Cyramza® (ramucirumab), Yervoy® (ipilmumab), Opdivo® (nivolumab), Braftovi® (encorafenib), Khapzory® (levoleucovorin) or Zirabev® (bevacizumab-bvzr).

Embodiment 31. The RTI for use of Embodiment 28 for the treatment of lung cancer, wherein the at least one second therapeutic agent is Etopophos® (etoposide), Hycamtin® (topotecan), VePesid® (etoposide), Toposar® (etoposide), Opdivo® (nivolumab), Keytruda® (pembrolizumab), Tecentriq® (atezolizumab), Imfinizi® (durvalumab), methotrexate, cyclophosphamide, Carboplatin, Cisplatin, docetaxel, Gemcitabine, Irinotecan, Paclitaxel, Pemetrexed, Vinblastine, or Vinorelbine.

Embodiment 32. The RTI for use of Embodiment 28 for the treatment of pancreatic ductal cancer, wherein the at least one second therapeutic agent is Gemzar® (Gemcitabine), fluorouracil, Afinitor® (everolimus), Tarceva® (erlotinib), Abraxane® (paclitaxel), capecitabine, Sutent® (sunitinib), pancreatin, methotrexate, Zanosar® (streptozocin), Mutamycin® (mitomycin), Onivyde® (irinotecan), bevacizumab, cetuximab, Infugem® (gemcitabine) or Lynparza® (olaparib).

Embodiment 33. The RTI for use of Embodiment 28 for the treatment of head and neck cancer, wherein the at least one second therapeutic agent is Erbituz® (cetuximab), Taxotere® (docetaxel), Trexall® (methotrexate), Keytruda® (pembrolizumab) or Opdivo® (nivolumab).

Embodiment 34. The RTI for use of Embodiment 28 for the treatment of prostate cancer, wherein the at least one second therapeutic agent is Suprefact® (buserelin), Firmagon® (degarelix), Zoladex® (goserelin), Vantas® (histrelin), Eligard® (leuprolide), Orgovyx® (relugolix), Trelstar® (triptorelin), Casodex® (bicalutamide), Eulexin® (flutamide), Nilandron® (nilutamide), Zytiga® (biraterone acetate), Erleada® (apalutamide), or Xtandi® (enzalutamide).

Embodiment 35. The RTI for use of Embodiment 28, wherein the at least one second therapeutic agent is a STING agonist.

Embodiment 36. The RTI for use of any one of Embodiments 1-35, wherein the patient is (a) not infected with the HIV virus, (b) not suspected of being infected with the HIV virus, (c) not being treated for the HIV virus, and/or (d) not being treated to prevent the HIV virus.

The disclosure also provides the following particular embodiments with respect to uses of a RTI in the manufacture of a medicament for treating cancer.

Embodiment 1. Use of a RTI in the manufacture of a medicament for treating cancer in patient in need thereof, wherein the RTI is to be administered according to an intermittent dosing schedule.

Embodiment 2. Use of a RTI in the manufacture of a medicament for increasing the expression of ORFp2 in the cancer cells of a patient, wherein the RTI is to be administered to the patient according to an intermittent dosing schedule.

Embodiment 3. The use of Embodiments 1 or 2, wherein the RTI is a nucleoside reverse transcriptase inhibitor (NRTI).

Embodiment 4. The use of Embodiments 1 or 2, wherein the RTI is a non-nucleoside reverse transcriptase inhibitor (NNRTI).

Embodiment 5. The use of Embodiments 1 or 2, wherein the RTI is a LINE-1 inhibitor.

Embodiment 6. The use of Embodiments 1 or 2, wherein the RTI is islatravir, elvucitabine, lamivudine (3TC), zidovudine (AZT), tenofovir, tenofovir disoproxil, tenofovir alafenamide, stavudine (d4T), zalcitabine (ddC), didanosine (ddI), emtricitabine (FTC), entecavir (ETV), 2′,3′-dideoxyguanosine (ddG), 2′,3′-dideoxyadenosine (ddA), 2′-fluoro-2′,3′-dideoxyarabinosyladenine (F-ddA), efavirenz (EFV), nevirapine (NVP), or abacavir (ABC), adefovir dipivoxil, or telbivudine.

Embodiment 7. The use of Embodiment 6, wherein the RTI is lamivudine, stavudine, emtricitabine, abacavir, tenofovir alafenamide, zidovudine, zalcitabine, didanosine, tenofovir disoproxil, adefovir dipivoxil, entecavir, or telbivudine.

Embodiment 8. The use of Embodiment 6, wherein the RTI is lamivudine.

Embodiment 9. The use of Embodiment 6, wherein the RTI is stavudine.

Embodiment 10. The use of Embodiment 6, wherein the RTI is emtricitabine.

Embodiment 11. The use of Embodiment 6, wherein the RTI is abacavir.

Embodiment 12. The use of Embodiment 6, wherein the RTI is tenofovir alafenamide.

Embodiment 13. The use of Embodiment 6, wherein the RTI is zidovudine.

Embodiment 14. The use of Embodiment 6, wherein the RTI is zalcitabine.

Embodiment 15. The use of Embodiment 6, wherein the RTI is didanosine.

Embodiment 16. The RTI for use of Embodiment 6, wherein the RTI is tenofovir disoproxil.

Embodiment 17. The use of Embodiment 6, wherein the RTI is adefovir dipivoxil.

Embodiment 18. The use of Embodiment 6, wherein the RTI is entecavir.

Embodiment 19. The use of Embodiment 6, wherein the RTI is telbivudine.

Embodiment 20. The use of any one of Embodiments 1-19, wherein the cancer is any one or more of the cancers of Table 1, e.g., breast cancer, colon cancer, lung cancer, pancreatic ductal cancer, prostate cancer, ovarian cancer, or head and neck cancer.

Embodiment 21. The use of Embodiment 20, wherein the cancer is breast cancer.

Embodiment 22. The use of Embodiment 20, wherein the cancer is colon cancer.

Embodiment 23. The use of Embodiment 20, wherein the cancer is lung cancer.

Embodiment 24. The use of Embodiment 20, wherein the cancer is pancreatic ductal cancer.

Embodiment 25. The use of Embodiment 20, wherein the cancer is prostate cancer.

Embodiment 26. The use of Embodiment 20, wherein the cancer is ovarian cancer.

Embodiment 27. The use of Embodiment 20, wherein the cancer is head and neck cancer.

Embodiment 28. The RTI for use of any one of Embodiments 1-27, further comprising administering a therapeutically effective amount of at least one second therapeutic agent useful for treating the cancer.

Embodiment 29. The use of Embodiment 28 for the treatment of breast cancer, wherein the at least one second therapeutic agent is Soltamox® (tamoxifen), Arimidex® (anastrozole), Femara® (letrozole), Aromasin® (exemestane), Herceptin® (trastuzumab), Abraxane® (paclitaxel), Cytoxan® (cyclophosphamide), Taxol® (paclitaxel), Afinitor® (everolimus), Taxotere® (docetaxel), Xeloda® (capecitabine), Trexall® (methotrexate), Faslodex (fulvestrant), Adriamycin® (doxorubicin), Perjeta® (pertuzumab), Gemzar (gemcitabine), Tykerb® (lapatinib), Adrucil® (fluorouracil), Ibrance® (palbociclib), Verzenio® (abemaciclib), Fareston® (toremifene), Halaven® (eribulin), Menest, Kadcyla® (ado-trastuzumab emtransine), Androxy® (fluoxymesterone), Avastin® (bevacizumab), esterified estrogens, Herzuma® (trastuzumab), Ixempra® (ixabepilone), Kanjinti® (trastuzumab), Kisqali® (ribociclib), Ogivri® (trastuzumab), Ontruzant® (trastuzumab), Tepadina® (thiotepa), Trazimera® (trastuzumab), Velban® (vinblastine), Piqray® (alpelisib), Tecentriq® (atezolizumab), Enhertu® (fam-trastuzumab deruxtecan), Herceptin, Hylecta™ (hyaluronidase/trastuzumab), Infugem® (gemcitabine), Kisqali® Femara® Co-Pack (ribociclib and letrozole), Talzenna® (talazoparib), Trodelvy® (sacituzumab) or Tukysa™ (tukatinib).

Embodiment 30. The use of Embodiment 28 for the treatment of colon cancer, wherein the at least one second therapeutic agent is Xeloda® (capecitabine), Eloxatin® (oxaliplatin), fluorouracil, Avastin® (bevacizumab), leucovorin, Camptosar® (irinotecan), Stivarga® (regorafenib), Erbitux® (cetuximab), Vectibix® (panitumumab), Lonsurf® (tipiracil/trifluridine), Zaltrap® (ziv-aflibercept), Betaseron® (interferon beta-1b), Fusilev® (levoleucovorin), Wellcocorin® (methotrexate), Keytruda® (pembrolizumab), Mvasi® (bevacizumab-awwb), Cyramza® (ramucirumab), Yervoy® (ipilmumab), Opdivo® (nivolumab), Braftovi® (encorafenib), Khapzory® (levoleucovorin) or Zirabev® (bevacizumab-bvzr).

Embodiment 31. The use of Embodiment 28 for the treatment of lung cancer, wherein the at least one second therapeutic agent is Etopophos® (etoposide), Hycamtin® (topotecan), VePesid® (etoposide), Toposar® (etoposide), Opdivo® (nivolumab), Keytruda® (pembrolizumab), Tecentriq® (atezolizumab), Imfinizi® (durvalumab), methotrexate, cyclophosphamide, Carboplatin, Cisplatin, docetaxel, Gemcitabine, Irinotecan, Paclitaxel, Pemetrexed, Vinblastine, or Vinorelbine.

Embodiment 32. The use of Embodiment 28 for the treatment of pancreatic ductal cancer, wherein the at least one second therapeutic agent is Gemzar® (Gemcitabine), fluorouracil, Afinitor® (everolimus), Tarceva® (erlotinib), Abraxane® (paclitaxel), capecitabine, Sutent® (sunitinib), pancreatin, methotrexate, Zanosar® (streptozocin), Mutamycin® (mitomycin), Onivyde® (irinotecan), bevacizumab, cetuximab, Infugem® (gemcitabine) or Lynparza® (olaparib).

Embodiment 33. The use of Embodiment 28 for the treatment of head and neck cancer, wherein the at least one second therapeutic agent is Erbituz® (cetuximab), Taxotere® (docetaxel), Trexall® (methotrexate), Keytruda® (pembrolizumab) or Opdivo® (nivolumab).

Embodiment 34. The use of Embodiment 28 for the treatment of prostate cancer, wherein the at least one second therapeutic agent is Suprefact® (buserelin), Firmagon® (degarelix), Zoladex® (goserelin), Vantas® (histrelin), Eligard® (leuprolide), Orgovyx® (relugolix), Trelstar® (triptorelin), Casodex® (bicalutamide), Eulexin® (flutamide), Nilandron® (nilutamide), Zytiga® (biraterone acetate), Erleada® (apalutamide), or Xtandi® (enzalutamide).

Embodiment 35. The use of Embodiment 28, wherein the at least one second therapeutic agent is a STING agonist.

Embodiment 36. The use of any one of Embodiments 1-35, wherein the patient is (a) not infected with the HIV virus, (b) not suspected of being infected with the HIV virus, (c) not being treated for the HIV virus, and/or (d) not being treated to prevent the HIV virus.

The disclosure also provides the following particular embodiments with respect to methods for treating cancer in a patient in need thereof.

Embodiment 1′. A method for treating cancer in patient in need thereof, the method comprising administering a therapeutically effective amount of a reverse transcriptase inhibitor (RTI) to the patient, wherein the RTI is administered according to a continuous or an intermittent dosing schedule.

Embodiment 2′. The method of Embodiment 1′, wherein the RTI is a nucleoside reverse transcriptase inhibitor (NRTI).

Embodiment 3′. The method of Embodiment 1′, wherein the RTI is a non-nucleoside reverse transcriptase inhibitor (NNRTI).

Embodiment 4′. The method of Embodiment 1′, wherein the RTI is a LINE-1 inhibitor.

Embodiment 5′. The method of Embodiment 1′, wherein the RTI is elvucitabine, lamivudine (3TC), zidovudine (AZT), tenofovir, tenofovir disoproxil, tenofovir alafenamide, stavudine (d4T), zalcitabine (ddC), didanosine (ddI), emtricitabine (FTC), entecavir (ETV), 2′,3′-dideoxyguanosine (ddG), 2′,3′-dideoxyadenosine (ddA), 2′-fluoro-2′,3′-dideoxyarabinosyladenine (F-ddA), efavirenz (EFV), nevirapine (NVP), or abacavir (ABC), adefovir dipivoxil, telbivudine, or islatravir.

Embodiment 6′. The method of Embodiment 5′, wherein the RTI is lamivudine, stavudine, emtricitabine, abacavir, tenofovir alafenamide, zidovudine, zalcitabine, didanosine, tenofovir disoproxil, adefovir dipivoxil, entecavir, telbivudine, or islatravir.

Embodiment 7′. The method of Embodiment 5′, wherein the RTI is lamivudine.

Embodiment 8′. The method of Embodiment 5′, wherein the RTI is stavudine.

Embodiment 9′. The method of Embodiment 5′, wherein the RTI is emtricitabine.

Embodiment 10′. The method of Embodiment 5′, wherein the RTI is abacavir.

Embodiment 11′. The method of Embodiment 5′, wherein the RTI is tenofovir alafenamide.

Embodiment 12′. The method of Embodiment 5′, wherein the RTI is zidovudine.

Embodiment 13′. The method of Embodiment 5′, wherein the RTI is zalcitabine.

Embodiment 14′. The method of Embodiment 5′, wherein the RTI is didanosine.

Embodiment 15′. The method of Embodiment 5′, wherein the RTI is tenofovir disoproxil.

Embodiment 16′. The method of Embodiment 5′, wherein the RTI is adefovir dipivoxil.

Embodiment 17′. The method of Embodiment 5′, wherein the RTI is entecavir.

Embodiment 18′. The method of Embodiment 5′, wherein the RTI is telbivudine.

Embodiment 19′. The method of Embodiment 5′, wherein the RTI is 2′-fluoro-2′,3′-dideoxyarabinosyladenine.

Embodiment 20′. The method of Embodiment 5′, wherein the RTI is islatravir.

Embodiment 21′. The method of Embodiment 1′, wherein the RTI is a compound of Formula I, see above, or a pharmaceutically acceptable salt or solvate thereof, or a tautomer thereof, wherein:

B is selected from the group consisting of B-1 and B-2, see above;

R¹ is selected from the group consisting of hydrogen and —OH;

R² is selected from the group consisting of methyl, ethynyl, and —CN;

R³ is selected from the group consisting of hydrogen, fluoro, chloro, bromo, iodo and methyl;

R⁴ is selected from the group consisting of —NH₂ and —OH;

R⁵ is selected from the group consisting of —NH₂ and —OH; and

R⁶ is selected from the group consisting of hydrogen, fluoro, chloro, and —NH₂.

Embodiment 22′. The method of Embodiment 21′, wherein the RTI is a compound of Formula II, see above, or a pharmaceutically acceptable salt or solvate thereof, or a tautomer thereof.

Embodiment 23′. The method of Embodiment 22′, wherein R³ is hydrogen.

Embodiment 24′. The method of Embodiment 22′, wherein R³ is selected from the group consisting of fluoro and chloro.

Embodiment 25′. The method of Embodiment 22′, wherein R³ is methyl.

Embodiment 26′. The method of any one of Embodiments 22′-25′, wherein R⁴ is —NH₂.

Embodiment 27′. The method of any one of Embodiments 22′-25′, wherein R⁴ is —OH.

Embodiment 28′. The method of Embodiment 21′, wherein the RTI is a compound is a compound of Formula III, see above, or a pharmaceutically acceptable salt or solvate thereof, or a tautomer thereof.

Embodiment 29′. The method of Embodiment 28′, wherein R⁵ is —NH₂.

Embodiment 30′. The method of Embodiment 28′, wherein R⁵ is —OH.

Embodiment 31′. The method of any one of Embodiments 28′-30′, wherein R⁶ is hydrogen.

Embodiment 32′. The method of any one of Embodiments 28′-30′, wherein R⁶ is chloro.

Embodiment 33′. The method of any one of Embodiments 28′-30′, wherein R⁶ is fluoro.

Embodiment 34′. The method of any one of Embodiments 28′-30′, wherein R⁶ is —NH₂.

Embodiment 35′. The method of any one of Embodiments 21′-34′, wherein R¹ is hydrogen.

Embodiment 36′. The method of any one of Embodiments 21′-34′, wherein R¹ is —OH.

Embodiment 37′. The method of any one of Embodiments 21′-36′, wherein R² is methyl.

Embodiment 38′. The method of any one of Embodiments 21′-36′, wherein R² is ethynyl.

Embodiment 39′. The method of any one of Embodiments 21′-36′, wherein R² is —CN.

Embodiment 40′. The method of Embodiment 1′, wherein the RTI is a compound of Table 3, or a pharmaceutically acceptable salt or solvate thereof, or a tautomer thereof.

Embodiment 41′. The method of Embodiment 1′, wherein the RTI is a compound of Table 4, or a pharmaceutically acceptable salt or solvate thereof, or a tautomer thereof.

Embodiment 42. The method of any one of Embodiments 1′-41′, wherein the cancer is breast cancer, colon cancer, lung cancer, pancreatic ductal cancer, prostate cancer, ovarian cancer, or head and neck cancer.

Embodiment 43′. The method of Embodiment 42′, wherein the cancer is breast cancer.

Embodiment 44′. The method of Embodiment 42′, wherein the cancer is colon cancer.

Embodiment 45′. The method of Embodiment 42′, wherein the cancer is lung cancer.

Embodiment 46′. The method of Embodiment 42′, wherein the cancer is pancreatic ductal cancer.

Embodiment 47′. The method of Embodiment 42′, wherein the cancer is prostate cancer.

Embodiment 48′. The method of Embodiment 47′, wherein the prostate cancer is high-risk localized prostate cancer.

Embodiment 49′. The method of Embodiment 42′, wherein the cancer is ovarian cancer.

Embodiment 50′. The method of Embodiment 42′, wherein the cancer is head and neck cancer.

Embodiment 51′. The method of any one of Embodiments 1′-41′, wherein the patient has prostate cancer and the RTI is administered as an adjuvant therapy.

Embodiment 52′. The method of any one of Embodiments 1′-41′, wherein the patient has prostate cancer and the RTI is administered as a neoadjuvant therapy.

Embodiment 53′. The method of any one of Embodiment 1′-52′, further comprising administering a therapeutically effective amount of at least one second therapeutic agent useful for treating the cancer.

Embodiment 54′. The method of Embodiment 53 for the treatment of breast cancer, wherein the at least one second therapeutic agent is Soltamox® (tamoxifen), Arimidex® (anastrozole), Femara® (letrozole), Aromasin® (exemestane), Herceptin® (trastuzumab), Abraxane® (paclitaxel), Cytoxan® (cyclophosphamide), Taxol® (paclitaxel), Afinitor® (everolimus), Taxotere® (docetaxel), Xeloda® (capecitabine), Trexall® (methotrexate), Faslodex (fulvestrant), Adriamycin® (doxorubicin), Perjeta® (pertuzumab), Gemzar (gemcitabine), Tykerb® (lapatinib), Adrucil® (fluorouracil), Ibrance® (palbociclib), Verzenio® (abemaciclib), Fareston® (toremifene), Halaven® (eribulin), Menest, Kadcyla® (ado-trastuzumab emtransine), Androxy® (fluoxymesterone), Avastin® (bevacizumab), esterified estrogens, Herzuma® (trastuzumab), Ixempra® (ixabepilone), Kanjinti® (trastuzumab), Kisqali® (ribociclib), Ogivri® (trastuzumab), Ontruzant® (trastuzumab), Tepadina® (thiotepa), Trazimera® (trastuzumab), Velban® (vinblastine), Piqray® (alpelisib), Tecentriq® (atezolizumab), Enhertu® (fam-trastuzumab deruxtecan), Herceptin, Hylecta™ (hyaluronidase/trastuzumab), Infugem® (gemcitabine), Kisqali® Femara® Co-Pack (ribociclib and letrozole), Talzenna® (talazoparib), Trodelvy® (sacituzumab) or Tukysa™ (tukatinib).

Embodiment 55′. The method of Embodiment 53 for the treatment of colon cancer, wherein the at least one second therapeutic agent is Xeloda® (capecitabine), Eloxatin® (oxaliplatin), fluorouracil, Avastin® (bevacizumab), leucovorin, Camptosar® (irinotecan), Stivarga® (regorafenib), Erbitux® (cetuximab), Vectibix® (panitumumab), Lonsurf® (tipiracil/trifluridine), Zaltrap® (ziv-aflibercept), Betaseron® (interferon beta-1b), Fusilev® (levoleucovorin), Wellcocorin® (methotrexate), Keytruda® (pembrolizumab), Mvasi® (bevacizumab-awwb), Cyramza® (ramucirumab), Yervoy® (ipilmumab), Opdivo® (nivolumab), Braftovi® (encorafenib), Khapzory® (levoleucovorin) or Zirabev® (bevacizumab-bvzr).

Embodiment 56′. The method of Embodiment 53 for the treatment of lung cancer, wherein the at least one second therapeutic agent is Etopophos® (etoposide), Hycamtin® (topotecan), VePesid® (etoposide), Toposar® (etoposide), Opdivo® (nivolumab), Keytruda® (pembrolizumab), Tecentriq® (atezolizumab), Imfinizi® (durvalumab), methotrexate, cyclophosphamide, Carboplatin, Cisplatin, docetaxel, Gemcitabine, Irinotecan, Paclitaxel, Pemetrexed, Vinblastine, or Vinorelbine.

Embodiment 57′. The method of Embodiment 53 for the treatment of pancreatic ductal cancer, wherein the at least one second therapeutic agent is Gemzar® (Gemcitabine), fluorouracil, Afinitor® (everolimus), Tarceva® (erlotinib), Abraxane® (paclitaxel), capecitabine, Sutent® (sunitinib), pancreatin, methotrexate, Zanosar® (streptozocin), Mutamycin® (mitomycin), Onivyde® (irinotecan), bevacizumab, cetuximab, Infugem® (gemcitabine) or Lynparza® (olaparib).

Embodiment 58′. The method of Embodiment 53 for the treatment of head and neck cancer, wherein the at least one second therapeutic agent is Erbituz® (cetuximab), Taxotere® (docetaxel), Trexall® (methotrexate), Keytruda® (pembrolizumab) or Opdivo® (nivolumab).

Embodiment 59′. The method of Embodiment 53 for the treatment of prostate cancer, wherein the at least one second therapeutic agent is Suprefact® (buserelin), Firmagon® (degarelix), Zoladex® (goserelin), Vantas® (histrelin), Eligard® (leuprolide), Orgovyx® (relugolix), Trelstar® (triptorelin), Casodex® (bicalutamide), Eulexin® (flutamide), Nilandron® (nilutamide), Zytiga® (biraterone acetate), Erleada® (apalutamide), or Xtandi® (enzalutamide).

Embodiment 60′. The method of Embodiment 53′, wherein the at least one second therapeutic agent is a STING agonist.

Embodiment 61′. The method of any one of Embodiments 1′-60′, wherein the patient is (a) not infected with the HIV virus, (b) not suspected of being infected with the HIV virus, (c) not being treated for the HIV virus, and/or (d) not being treated to prevent the HIV virus.

Embodiment 62′ The method of any one of Embodiments 1′-61′, wherein the RTI is administered according to a continuous dosing schedule.

Embodiment 63′ The method of any one of Embodiments 1′-61′, wherein the RTI is administered according to an intermittent dosing schedule.

Embodiment 64′. A kit for carrying out the method of any one Embodiments 1′-63′, the kit comprising (i) a RTI; and (ii) and instructions for administering the RTI to a patient having cancer.

Embodiment 65′. The kit of Embodiment 64′ further comprising at least one second therapeutic agent.

The disclosure also provides the following particular embodiments with respect to RTIs (and compositions thereof) for use to treat cancer in a subject.

Embodiment 1′. A RTI for use in treating cancer in patient in need thereof, wherein the RTI is to be administered according to a continuous or an intermittent dosing schedule.

Embodiment 2′. The RTI for use of Embodiment 1′, wherein the RTI is a nucleoside reverse transcriptase inhibitor (NRTI).

Embodiment 3′. The RTI for use of Embodiment 1′, wherein the RTI is a non-nucleoside reverse transcriptase inhibitor (NNRTI).

Embodiment 4′. The RTI for use of Embodiment 1′, wherein the RTI is a LINE-1 inhibitor.

Embodiment 5′. The RTI for use of Embodiment 1′, wherein the RTI is elvucitabine, lamivudine (3TC), zidovudine (AZT), tenofovir, tenofovir disoproxil, tenofovir alafenamide, stavudine (d4T), zalcitabine (ddC), didanosine (ddI), emtricitabine (FTC), entecavir (ETV), 2′,3′-dideoxyguanosine (ddG), 2′,3′-dideoxyadenosine (ddA), 2′-fluoro-2′,3′-dideoxyarabinosyladenine (F-ddA), efavirenz (EFV), nevirapine (NVP), or abacavir (ABC), adefovir dipivoxil, telbivudine, or islatravir.

Embodiment 6′. The RTI for use of Embodiment 5′, wherein the RTI is lamivudine, stavudine, emtricitabine, abacavir, tenofovir alafenamide, zidovudine, zalcitabine, didanosine, tenofovir disoproxil, adefovir dipivoxil, entecavir, telbivudine, or islatravir.

Embodiment 7′. The RTI for use of Embodiment 5′, wherein the RTI is lamivudine.

Embodiment 8′. The RTI for use of Embodiment 5′, wherein the RTI is stavudine.

Embodiment 9′. The RTI for use of Embodiment 5′, wherein the RTI is emtricitabine.

Embodiment 10′. The RTI for use of Embodiment 5′, wherein the RTI is abacavir.

Embodiment 11′. The RTI for use of Embodiment 5′, wherein the RTI is tenofovir alafenamide.

Embodiment 12′. The RTI for use of Embodiment 5′, wherein the RTI is zidovudine.

Embodiment 13′. The RTI for use of Embodiment 5′, wherein the RTI is zalcitabine.

Embodiment 14′. The RTI for use of Embodiment 5′, wherein the RTI is didanosine.

Embodiment 15′. The RTI for use of Embodiment 5′, wherein the RTI is tenofovir disoproxil.

Embodiment 16′. The RTI for use of Embodiment 5′, wherein the RTI is adefovir dipivoxil.

Embodiment 17′. The RTI for use of Embodiment 5′, wherein the RTI is entecavir.

Embodiment 18′. The RTI for use of Embodiment 5′, wherein the RTI is telbivudine.

Embodiment 19′. The RTI for use of Embodiment 5′, wherein the RTI is 2′-fluoro-2′,3′-dideoxyarabinosyladenine.

Embodiment 20′. The RTI for use of Embodiment 5′, wherein the RTI is islatravir.

Embodiment 21′. The RTI for use of Embodiment 1′, wherein the RTI is a compound of Formula, see above, or a pharmaceutically acceptable salt or solvate thereof, or a tautomer thereof, wherein:
B is selected from the group consisting of B-1 and B-2, see above;
R¹ is selected from the group consisting of hydrogen and —OH;
R² is selected from the group consisting of methyl, ethynyl, and —CN;
R³ is selected from the group consisting of hydrogen, fluoro, chloro, bromo, iodo and methyl;
R⁴ is selected from the group consisting of —NH₂ and —OH;
R⁵ is selected from the group consisting of —NH₂ and —OH; and
R⁶ is selected from the group consisting of hydrogen, fluoro, chloro, and —NH₂.

Embodiment 22′. The method of Embodiment 21′, wherein the RTI is a compound of Formula II see above, or a pharmaceutically acceptable salt or solvate thereof, or a tautomer thereof.

Embodiment 23′. The RTI for use of Embodiment 22′, wherein R³ is hydrogen.

Embodiment 24′. The RTI for use of f Embodiment 22′, wherein R³ is selected from the group consisting of fluoro and chloro.

Embodiment 25′. The RTI for use of Embodiment 22′, wherein R³ is methyl.

Embodiment 26′. The RTI for use of any one of Embodiments 22′-25′, wherein R⁴ is —NH₂.

Embodiment 27′. The RTI for use of any one of Embodiments 22′-25′, wherein R⁴ is —OH.

Embodiment 28′. The RTI for use of Embodiment 21′, wherein the RTI is a compound is a compound of Formula III see above, or a pharmaceutically acceptable salt or solvate thereof, or a tautomer thereof.

Embodiment 29′. The RTI for use of Embodiment 28′, wherein R⁵ is —NH₂.

Embodiment 30′. The RTI for use of Embodiment 28′, wherein R⁵ is —OH.

Embodiment 31′. The RTI for use of any one of Embodiments 28′-30′, wherein R⁶ is hydrogen.

Embodiment 32′. The RTI for use of any one of Embodiments 28′-30′, wherein R⁶ is chloro.

Embodiment 33′. The RTI for use of any one of Embodiments 28′-30′, wherein R⁶ is fluoro.

Embodiment 34′. The RTI for use of any one of Embodiments 28′-30′, wherein R⁶ is —NH₂.

Embodiment 35′. The RTI for use of any one of Embodiments 21′-34′, wherein R¹ is hydrogen.

Embodiment 36′. The RTI for use of any one of Embodiments 21′-34′, wherein R¹ is —OH.

Embodiment 37′. The RTI for use of any one of Embodiments 21′-36′, wherein R² is methyl.

Embodiment 38′. The RTI for use of any one of Embodiments 21′-36′, wherein R² is ethynyl.

Embodiment 39′. The RTI for use of any one of Embodiments 21′-36′, wherein R² is —CN.

Embodiment 40′. The RTI for use of Embodiment 1′, wherein the RTI is a compound of Table 3, or a pharmaceutically acceptable salt or solvate thereof, or a tautomer thereof.

Embodiment 41′. The RTI for use of Embodiment 1′, wherein the RTI is a compound of Table 4, or a pharmaceutically acceptable salt or solvate thereof, or a tautomer thereof.

Embodiment 42. The RTI for use of any one of Embodiments 1′-41′, wherein the cancer is breast cancer, colon cancer, lung cancer, pancreatic ductal cancer, prostate cancer, ovarian cancer, or head and neck cancer.

Embodiment 43′. The RTI for use of Embodiment 42′, wherein the cancer is breast cancer.

Embodiment 44′. The RTI for use of Embodiment 42′, wherein the cancer is colon cancer.

Embodiment 45′. The RTI for use of Embodiment 42′, wherein the cancer is lung cancer.

Embodiment 46′. The RTI for use of Embodiment 42′, wherein the cancer is pancreatic ductal cancer.

Embodiment 47′. The RTI for use of Embodiment 42′, wherein the cancer is prostate cancer.

Embodiment 48′. The RTI for use of Embodiment 47′, wherein the prostate cancer is high-risk localized prostate cancer.

Embodiment 49′. The RTI for use of Embodiment 42′, wherein the cancer is ovarian cancer.

Embodiment 50′. The RTI for use of Embodiment 42′, wherein the cancer is head and neck cancer.

Embodiment 51′. The RTI for use of any one of Embodiments 1′-41′, wherein the patient has prostate cancer and the RTI is administered as an adjuvant therapy.

Embodiment 52′. The RTI for use of any one of Embodiments 1′-41′, wherein the patient has prostate cancer and the RTI is administered as a neoadjuvant therapy.

Embodiment 53′. The RTI for use of any one of Embodiment 1′-52′, further comprising administering a therapeutically effective amount of at least one second therapeutic agent useful for treating the cancer.

Embodiment 54′. The RTI for use of Embodiment 53′ for the treatment of breast cancer, wherein the at least one second therapeutic agent is Soltamox® (tamoxifen), Arimidex® (anastrozole), Femara® (letrozole), Aromasin® (exemestane), Herceptin® (trastuzumab), Abraxane® (paclitaxel), Cytoxan® (cyclophosphamide), Taxol® (paclitaxel), Afinitor® (everolimus), Taxotere® (docetaxel), Xeloda® (capecitabine), Trexall® (methotrexate), Faslodex (fulvestrant), Adriamycin® (doxorubicin), Perjeta® (pertuzumab), Gemzar (gemcitabine), Tykerb® (lapatinib), Adrucil® (fluorouracil), Ibrance® (palbociclib), Verzenio® (abemaciclib), Fareston® (toremifene), Halaven® (eribulin), Menest, Kadcyla® (ado-trastuzumab emtransine), Androxy® (fluoxymesterone), Avastin® (bevacizumab), esterified estrogens, Herzuma® (trastuzumab), Ixempra® (ixabepilone), Kanjinti® (trastuzumab), Kisqali® (ribociclib), Ogivri® (trastuzumab), Ontruzant® (trastuzumab), Tepadina® (thiotepa), Trazimera® (trastuzumab), Velban® (vinblastine), Piqray® (alpelisib), Tecentriq® (atezolizumab), Enhertu® (fam-trastuzumab deruxtecan), Herceptin, Hylecta™ (hyaluronidase/trastuzumab), Infugem® (gemcitabine), Kisqali® Femara® Co-Pack (ribociclib and letrozole), Talzenna® (talazoparib), Trodelvy® (sacituzumab) or Tukysa™ (tukatinib).

Embodiment 55′. The RTI for use of Embodiment 53′ for the treatment of colon cancer, wherein the at least one second therapeutic agent is Xeloda® (capecitabine), Eloxatin® (oxaliplatin), fluorouracil, Avastin® (bevacizumab), leucovorin, Camptosar® (irinotecan), Stivarga® (regorafenib), Erbitux® (cetuximab), Vectibix® (panitumumab), Lonsurf® (tipiracil/trifluridine), Zaltrap® (ziv-aflibercept), Betaseron® (interferon beta-1b), Fusilev® (levoleucovorin), Wellcocorin® (methotrexate), Keytruda® (pembrolizumab), Mvasi® (bevacizumab-awwb), Cyramza® (ramucirumab), Yervoy® (ipilmumab), Opdivo® (nivolumab), Braftovi® (encorafenib), Khapzory® (levoleucovorin) or Zirabev® (bevacizumab-bvzr).

Embodiment 56′. The method of Embodiment 53′ for the treatment of lung cancer, wherein the at least one second therapeutic agent is Etopophos® (etoposide), Hycamtin® (topotecan), VePesid® (etoposide), Toposar® (etoposide), Opdivo® (nivolumab), Keytruda® (pembrolizumab), Tecentriq® (atezolizumab), Imfinizi® (durvalumab), methotrexate, cyclophosphamide, Carboplatin, Cisplatin, docetaxel, Gemcitabine, Irinotecan, Paclitaxel, Pemetrexed, Vinblastine, or Vinorelbine.

Embodiment 57′. The RTI for use of Embodiment 53 for the treatment of pancreatic ductal cancer, wherein the at least one second therapeutic agent is Gemzar® (Gemcitabine), fluorouracil, Afinitor® (everolimus), Tarceva® (erlotinib), Abraxane® (paclitaxel), capecitabine, Sutent® (sunitinib), pancreatin, methotrexate, Zanosar® (streptozocin), Mutamycin® (mitomycin), Onivyde® (irinotecan), bevacizumab, cetuximab, Infugem® (gemcitabine) or Lynparza® (olaparib).

Embodiment 58′. The method of Embodiment 53′ for the treatment of head and neck cancer, wherein the at least one second therapeutic agent is Erbituz® (cetuximab), Taxotere® (docetaxel), Trexall® (methotrexate), Keytruda® (pembrolizumab) or Opdivo® (nivolumab).

Embodiment 59′. The RTI for use of Embodiment 53′ for the treatment of prostate cancer, wherein the at least one second therapeutic agent is Suprefact® (buserelin), Firmagon® (degarelix), Zoladex® (goserelin), Vantas® (histrelin), Eligard® (leuprolide), Orgovyx® (relugolix), Trelstar® (triptorelin), Casodex® (bicalutamide), Eulexin® (flutamide), Nilandron® (nilutamide), Zytiga® (biraterone acetate), Erleada® (apalutamide), or Xtandi® (enzalutamide).

Embodiment 60′. The RTI for use of Embodiment 53′, wherein the at least one second therapeutic agent is a STING agonist.

Embodiment 61′. The RTI for use of any one of Embodiments 1′-60′, wherein the patient is (a) not infected with the HIV virus, (b) not suspected of being infected with the HIV virus, (c) not being treated for the HIV virus, and/or (d) not being treated to prevent the HIV virus.

Embodiment 62′ The RTI for use of any one of Embodiments 1′-61′, wherein the RTI is to be administered according to a continuous dosing schedule.

Embodiment 63′ The RTI for use of any one of Embodiments 1′-61′, wherein the RTI is to be administered according to an intermittent dosing schedule.

The disclosure also provides the following particular embodiments with respect to uses of a RTI in the manufacture of a medicament for treating cancer.

Embodiment 1′. Use of a RTI in the manufacture of a medicament for treating cancer in patient in need thereof, wherein the RTI is to be administered according to a continuous or an intermittent dosing schedule.

Embodiment 2′. The use of Embodiment 1′, wherein the RTI is a nucleoside reverse transcriptase inhibitor (NRTI).

Embodiment 3′. The use of Embodiment 1′, wherein the RTI is a non-nucleoside reverse transcriptase inhibitor (NNRTI).

Embodiment 4′. The use of Embodiment 1′, wherein the RTI is a LINE-1 inhibitor.

Embodiment 5′. The use of Embodiment 1′, wherein the RTI is elvucitabine, lamivudine (3TC), zidovudine (AZT), tenofovir, tenofovir disoproxil, tenofovir alafenamide, stavudine (d4T), zalcitabine (ddC), didanosine (ddI), emtricitabine (FTC), entecavir (ETV), 2′,3′-dideoxyguanosine (ddG), 2′,3′-dideoxyadenosine (ddA), 2′-fluoro-2′,3′-dideoxyarabinosyladenine (F-ddA), efavirenz (EFV), nevirapine (NVP), or abacavir (ABC), adefovir dipivoxil, telbivudine, or islatravir.

Embodiment 6′. The use of Embodiment 5′, wherein the RTI is lamivudine, stavudine, emtricitabine, abacavir, tenofovir alafenamide, zidovudine, zalcitabine, didanosine, tenofovir disoproxil, adefovir dipivoxil, entecavir, telbivudine, or islatravir.

Embodiment 7′. The use of Embodiment 5′, wherein the RTI is lamivudine.

Embodiment 8′. The use of Embodiment 5′, wherein the RTI is stavudine.

Embodiment 9′. The use of Embodiment 5′, wherein the RTI is emtricitabine.

Embodiment 10′. The use of Embodiment 5′, wherein the RTI is abacavir.

Embodiment 11′. The use of Embodiment 5′, wherein the RTI is tenofovir alafenamide.

Embodiment 12′. The use of Embodiment 5′, wherein the RTI is zidovudine.

Embodiment 13′. The use of Embodiment 5′, wherein the RTI is zalcitabine.

Embodiment 14′. The use of Embodiment 5′, wherein the RTI is didanosine.

Embodiment 15′. The use of Embodiment 5′, wherein the RTI is tenofovir disoproxil.

Embodiment 16′. The use of Embodiment 5′, wherein the RTI is adefovir dipivoxil.

Embodiment 17′. The use of Embodiment 5′, wherein the RTI is entecavir.

Embodiment 18′. The use of Embodiment 5′, wherein the RTI is telbivudine.

Embodiment 19′. The use of Embodiment 5′, wherein the RTI is 2′-fluoro-2′,3′-dideoxyarabinosyladenine.

Embodiment 20′. The use of Embodiment 5′, wherein the RTI is islatravir.

Embodiment 21′. The use of Embodiment 1′, wherein the RTI is a compound of Formula I, see above, or a pharmaceutically acceptable salt or solvate thereof, or a tautomer thereof, wherein:
B is selected from the group consisting of B-1 and B-2, see above;
R¹ is selected from the group consisting of hydrogen and —OH;
R² is selected from the group consisting of methyl, ethynyl, and —CN;
R³ is selected from the group consisting of hydrogen, fluoro, chloro, bromo, iodo and methyl;
R⁴ is selected from the group consisting of —NH₂ and —OH;
R⁵ is selected from the group consisting of —NH₂ and —OH; and
R⁶ is selected from the group consisting of hydrogen, fluoro, chloro, and —NH₂.

Embodiment 22′. The method of Embodiment 21′, wherein the RTI is a compound of Formula II, see above, or a pharmaceutically acceptable salt or solvate thereof, or a tautomer thereof.

Embodiment 23′. The use of Embodiment 22′, wherein R³ is hydrogen.

Embodiment 24′. The use of Embodiment 22′, wherein R³ is selected from the group consisting of fluoro and chloro.

Embodiment 25′. The use of Embodiment 22′, wherein R³ is methyl.

Embodiment 26′. The use of any one of Embodiments 22′-25′, wherein R⁴ is —NH₂.

Embodiment 27′. The use of any one of Embodiments 22′-25′, wherein R⁴ is —OH.

Embodiment 28′. The use of Embodiment 21′, wherein the RTI is a compound is a compound of Formula III, see above, or a pharmaceutically acceptable salt or solvate thereof, or a tautomer thereof.

Embodiment 29′. The use of Embodiment 28′, wherein R⁵ is —NH₂.

Embodiment 30′. The use of Embodiment 28′, wherein R⁵ is —OH.

Embodiment 31′. The use of any one of Embodiments 28′-30′, wherein R⁶ is hydrogen.

Embodiment 32′. The use of any one of Embodiments 28′-30′, wherein R⁶ is chloro.

Embodiment 33′. The use of any one of Embodiments 28′-30′, wherein R⁶ is fluoro.

Embodiment 34′. The use of any one of Embodiments 28′-30′, wherein R⁶ is —NH₂.

Embodiment 35′. The use of any one of Embodiments 21′-34′, wherein R¹ is hydrogen.

Embodiment 36′. The use of any one of Embodiments 21′-34′, wherein R¹ is —OH.

Embodiment 37′. The use of any one of Embodiments 21′-36′, wherein R² is methyl.

Embodiment 38′. The use of any one of Embodiments 21′-36′, wherein R² is ethynyl.

Embodiment 39′. The use of any one of Embodiments 21′-36′, wherein R² is —CN.

Embodiment 40′. The use of Embodiment 1′, wherein the RTI is a compound of Table 3, or a pharmaceutically acceptable salt or solvate thereof, or a tautomer thereof.

Embodiment 41′. The use of Embodiment 1′, wherein the RTI is a compound of Table 4, or a pharmaceutically acceptable salt or solvate thereof, or a tautomer thereof.

Embodiment 42′. The use of any one of Embodiments 1′-41′, wherein the cancer is breast cancer, colon cancer, lung cancer, pancreatic ductal cancer, prostate cancer, ovarian cancer, or head and neck cancer.

Embodiment 43′. The use of Embodiment 42′, wherein the cancer is breast cancer.

Embodiment 44′. The use of Embodiment 42′, wherein the cancer is colon cancer.

Embodiment 45′. The use of Embodiment 42′, wherein the cancer is lung cancer.

Embodiment 46′. The use of Embodiment 42′, wherein the cancer is pancreatic ductal cancer.

Embodiment 47′. The use of Embodiment 42′, wherein the cancer is prostate cancer.

Embodiment 48′. The use of Embodiment 47′, wherein the prostate cancer is high-risk localized prostate cancer.

Embodiment 49′. The use of Embodiment 42′, wherein the cancer is ovarian cancer.

Embodiment 50′. The use of Embodiment 42′, wherein the cancer is head and neck cancer.

Embodiment 51′. The use of any one of Embodiments 1′-41′, wherein the patient has prostate cancer and the RTI is administered as an adjuvant therapy.

Embodiment 52′. The use of any one of Embodiments 1′-41′, wherein the patient has prostate cancer and the RTI is administered as a neoadjuvant therapy.

Embodiment 53′. The use of any one of Embodiment 1′-52′, further comprising administering a therapeutically effective amount of at least one second therapeutic agent useful for treating the cancer.

Embodiment 54′. The use of Embodiment 53′ for the treatment of breast cancer, wherein the at least one second therapeutic agent is Soltamox® (tamoxifen), Arimidex® (anastrozole), Femara® (letrozole), Aromasin® (exemestane), Herceptin® (trastuzumab), Abraxane® (paclitaxel), Cytoxan® (cyclophosphamide), Taxol® (paclitaxel), Afinitor® (everolimus), Taxotere® (docetaxel), Xeloda® (capecitabine), Trexall® (methotrexate), Faslodex (fulvestrant), Adriamycin® (doxorubicin), Perjeta® (pertuzumab), Gemzar (gemcitabine), Tykerb® (lapatinib), Adrucil® (fluorouracil), Ibrance® (palbociclib), Verzenio® (abemaciclib), Fareston® (toremifene), Halaven® (eribulin), Menest, Kadcyla® (ado-trastuzumab emtransine), Androxy® (fluoxymesterone), Avastin® (bevacizumab), esterified estrogens, Herzuma® (trastuzumab), Ixempra® (ixabepilone), Kanjinti® (trastuzumab), Kisqali® (ribociclib), Ogivri® (trastuzumab), Ontruzant® (trastuzumab), Tepadina® (thiotepa), Trazimera® (trastuzumab), Velban® (vinblastine), Piqray® (alpelisib), Tecentriq® (atezolizumab), Enhertu® (fam-trastuzumab deruxtecan), Herceptin, Hylecta™ (hyaluronidase/trastuzumab), Infugem® (gemcitabine), Kisqali® Femara® Co-Pack (ribociclib and letrozole), Talzenna® (talazoparib), Trodelvy® (sacituzumab) or Tukysa™ (tukatinib).

Embodiment 55′. The use of Embodiment 53′ for the treatment of colon cancer, wherein the at least one second therapeutic agent is Xeloda® (capecitabine), Eloxatin® (oxaliplatin), fluorouracil, Avastin® (bevacizumab), leucovorin, Camptosar® (irinotecan), Stivarga® (regorafenib), Erbitux® (cetuximab), Vectibix® (panitumumab), Lonsurf® (tipiracil/trifluridine), Zaltrap® (ziv-aflibercept), Betaseron® (interferon beta-1b), Fusilev® (levoleucovorin), Wellcocorin® (methotrexate), Keytruda® (pembrolizumab), Mvasi® (bevacizumab-awwb), Cyramza® (ramucirumab), Yervoy® (ipilmumab), Opdivo® (nivolumab), Braftovi® (encorafenib), Khapzory® (levoleucovorin) or Zirabev® (bevacizumab-bvzr).

Embodiment 56′. The method of Embodiment 53′ for the treatment of lung cancer, wherein the at least one second therapeutic agent is Etopophos® (etoposide), Hycamtin® (topotecan), VePesid® (etoposide), Toposar® (etoposide), Opdivo® (nivolumab), Keytruda® (pembrolizumab), Tecentriq® (atezolizumab), Imfinizi® (durvalumab), methotrexate, cyclophosphamide, Carboplatin, Cisplatin, docetaxel, Gemcitabine, Irinotecan, Paclitaxel, Pemetrexed, Vinblastine, or Vinorelbine.

Embodiment 57′. The use of Embodiment 53 for the treatment of pancreatic ductal cancer, wherein the at least one second therapeutic agent is Gemzar® (Gemcitabine), fluorouracil, Afinitor® (everolimus), Tarceva® (erlotinib), Abraxane® (paclitaxel), capecitabine, Sutent® (sunitinib), pancreatin, methotrexate, Zanosar® (streptozocin), Mutamycin® (mitomycin), Onivyde® (irinotecan), bevacizumab, cetuximab, Infugem® (gemcitabine) or Lynparza® (olaparib).

Embodiment 58′. The method of Embodiment 53′ for the treatment of head and neck cancer, wherein the at least one second therapeutic agent is Erbituz® (cetuximab), Taxotere® (docetaxel), Trexall® (methotrexate), Keytruda® (pembrolizumab) or Opdivo® (nivolumab).

Embodiment 59′. The use of Embodiment 53′ for the treatment of prostate cancer, wherein the at least one second therapeutic agent is Suprefact® (buserelin), Firmagon® (degarelix), Zoladex® (goserelin), Vantas® (histrelin), Eligard® (leuprolide), Orgovyx® (relugolix), Trelstar® (triptorelin), Casodex® (bicalutamide), Eulexin® (flutamide), Nilandron® (nilutamide), Zytiga® (biraterone acetate), Erleada® (apalutamide), or Xtandi® (enzalutamide).

Embodiment 60′. The use of Embodiment 53′, wherein the at least one second therapeutic agent is a STING agonist.

Embodiment 61′. The use of any one of Embodiments 1′-60′, wherein the patient is (a) not infected with the HIV virus, (b) not suspected of being infected with the HIV virus, (c) not being treated for the HIV virus, and/or (d) not being treated to prevent the HIV virus.

Embodiment 62′ The use of any one of Embodiments 1′-61′, wherein the RTI is to be administered according to a continuous dosing schedule.

Embodiment 63′ The use of any one of Embodiments 1′-61′, wherein the RTI is to be administered according to an intermittent dosing schedule.

The disclosure also provides the following particular embodiments with respect to methods of treating cancer in a patient with representative compounds, e.g., LINE-1 inhibitors, of the disclosure.

Embodiment I. A method for treating cancer in patient in need thereof, the method comprising administering a therapeutically effective amount of Cpd. No. 7, Cpd. No. 9, Cpd. No. 13, Cpd. No. 15, Cpd. No. 20, Cpd. No. 21, Cpd. No. 25, Cpd. No. 25, Cpd. No. 30, Cpd. No. 31, Cpd. No. 32, islatravir, or elvucitabine, to the patient.

Embodiment II. The method of Embodiment I comprising administering a therapeutically effective amount of Cpd. No. 29 to the patient.

Embodiment III. The method of Embodiment I comprising administering a therapeutically effective amount of Cpd. No. 32 to the patient

Embodiment IV. The method of any one of Embodiments I-III, wherein the cancer is breast cancer, colon cancer, lung cancer, pancreatic ductal cancer, prostate cancer, ovarian cancer, or head and neck cancer.

Embodiment V. The method of Embodiment IV, wherein the cancer is breast cancer.

Embodiment VI. The method of Embodiment IV, wherein the cancer is colon cancer.

Embodiment VII. The method of Embodiment IV, wherein the cancer is lung cancer.

Embodiment VIII. The method of Embodiment IV, wherein the cancer is pancreatic ductal cancer.

Embodiment IX. The method of Embodiment IV, wherein the cancer is prostate cancer.

Embodiment X. The method of Embodiment IV, wherein the cancer is ovarian cancer.

Embodiment XI. The method of Embodiment IV, wherein the cancer is head and neck cancer.

Embodiment XII. The method of any one Embodiments I-XI, further comprising administering a therapeutically effective amount of at least one second therapeutic agent useful for treating the cancer.

Embodiment XIII. The method of Embodiment XII for treating breast cancer, wherein the at least one second therapeutic agent is tamoxifen, anastrozole, letrozole, exemestane, trastuzumab, paclitaxel, cyclophosphamide, everolimus, docetaxel, capecitabine, methotrexate, fulvestrant, doxorubicin, pertuzumab, gemcitabine, lapatinib, fluorouracil, palbociclib, abemaciclib, toremifene, eribulin, ado-trastuzumab emtransine, fluoxymesterone, bevacizumab, esterified estrogens, ixabepilone, ribociclib, thiotepa, vinblastine, alpelisib, atezolizumab, fam-trastuzumab deruxtecan, hyaluronidase/trastuzumab, ribociclib/letrozole, talazoparib, sacituzumab or tukatinib.

Embodiment XIV. The method of Embodiment XII for treating colon cancer, wherein the at least one second therapeutic agent is capecitabine, oxaliplatin, fluorouracil, bevacizumab, leucovorin, irinotecan, regorafenib, cetuximab, panitumumab, tipiracil/trifluridine, ziv-aflibercept, interferon beta-1b, methotrexate, pembrolizumab, bevacizumab-awwb, ramucirumab, ipilmumab, nivolumab, encorafenib, levoleucovorin or bevacizumab-bvzr.

Embodiment XV. The method of Embodiment XII for treating lung cancer, wherein the at least one second therapeutic agent is etoposide, topotecan, nivolumab, pembrolizumab, atezolizumab, durvalumab, methotrexate, cyclophosphamide, carboplatin, cisplatin, docetaxel, gemcitabine, irinotecan, paclitaxel, pemetrexed, vinblastine, or vinorelbine.

Embodiment XVI. The method of Embodiment XII for treating pancreatic ductal cancer, wherein the at least one second therapeutic agent is gemcitabine, fluorouracil, everolimus, erlotinib, paclitaxel, capecitabine, sunitinib, pancreatin, methotrexate, streptozocin, mitomycin, irinotecan, bevacizumab, cetuximab or olaparib.

Embodiment XVII. The method of Embodiment XII for treating head and neck cancer, wherein the at least one second therapeutic agent is cetuximab, docetaxel, methotrexate, pembrolizumab or nivolumab.

Embodiment XVIII. The method of Embodiment XII for treating prostate cancer, wherein the at least one second therapeutic agent is buserelin, degarelix, goserelin, histrelin, leuprolide, relugolix, triptorelin, bicalutamide, flutamide, nilutamide, biraterone acetate, apalutamide, or enzalutamide.

Embodiment XIX. The method of any one of Embodiments I-XVIII, wherein the patient is (a) not infected with the HIV virus, (b) not suspected of being infected with the HIV virus, (c) not being treated for the HIV virus, and/or (d) not being treated to prevent the HIV virus.

Embodiment XX. A kit for carrying out the method of any one of Embodiments I-XIX, the kit comprising (i) the compound; and (ii) and instructions for administering the compound to a patient having cancer.

### Example 1

Representative compounds were tested for inhibition of retrotransposition activity of human LINE-1 (L1) retrotransposition reporter assay in HeLa cells according to the following procedure.

Human Cervical Adenocarcinoma Cells (HeLa) were cultured in Dulbecco's Modified Eagle's Medium (DMEM) containing 4500 mg/L glucose, L-glutamine, sodium pyruvate and sodium bicarbonate, supplemented with 10% of heat inactivated fetal bovine serum (FBS). Cells were grown at 37° C. in an atmosphere of 5% CO₂. The dual luciferase-encoding plasmid pYX017 was provided by Prof Wenfeng An (Xie, et al., 2011, Nucleic Acids Res).

The reporter assay was performed in 96-well white, optical bottom plates (Thermo Fisher, 165306). 24 hours prior to transfection, HeLa cells were seeded in a density of 2,500 cells per well, in 50 μL of DMEM, 10% FBS media volume. Cells were incubated at 37° C. in an atmosphere of 5% CO2. Seeding was optimized to achieve approximately 30% cell confluency on the day of transfection. A transfection mix was prepared by combining transfection reagent FuGENE® HD (Promega, E2311) and plasmid DNA (100 ng per well) in a 3:1 ratio, in OpiMEM media (Thermo Fisher, 31985062). Next, 5 μL of the transfection mix was combined with 20 μL of DMEM, 10% FBS media, and 25 μL of this mix was added into each well of an assay plate. In each assay plate one column remained untransfected to serve as a blank luminescence signal.

Serial dilution of test compounds was performed in DMSO and transferred to medium. Next, 25 μL of media-diluted compounds series were transferred to an assay plate containing cells and transfection media. The final concentration of DMSO in treated wells was 0.2%. Serial dilutions of compounds were added to an assay plate immediately after transfection. Compounds were tested in triplicates. The assay plate was then incubated with transfection reagent and compounds at 37° C. in an atmosphere of 5% CO₂ for 72 hours.

Luciferase reporter activity was quantified with the Dual-Luciferase® Reporter Assay System (Promega) following the manufacturer's manual, with a passive lysis buffer volume increased to 30 μL and lysis incubation time increased to 20 min at room temperature with gentle shaking, to ensure complete cell lysis. Luminescence was measured using a SpectraMax i3×Multi-Mode Microplate Reader, with integration times of 100 ms and 10 ms applied to measure FLuc and RLuc signals, respectively. Relative LNE-1 activity was calculated as Firefly signal/Renilla signal*10,000. In order to determine IC₅₀ values for each compound, dose response data were fit to a logistic using non-linear regression.

The results for representative compounds of the disclosure are provided in Table 5.

TABLE 5
Human L1 activity inhibition

| | Human LINE-1 | | Human LINE-1 |
|---|---|---|---|
| Cpd. | IC₅₀ (μM) | Name | IC₅₀ (μM) |
| 2 | 0.39 | Islatravir (EFdA) | 0.0011 |
| 4 | 0.49 | Zalcitabine | 0.066 |
| 6 | 18.56 | Censavudine | 0.070 |
| 7 | 0.0097 | Elvucitabine | 0.092 |
| 9 | 0.021 | Emtricitabine (FTC) | 0.48 |
| 12 | 0.0062 | Tenofovir Disoproxil | 0.19 |
| 13 | 0.00051 | Didanosine | 0.53 |
| 15 | 0.8333 | AZT | 0.63 |
| 16 | >25 | Lamivudine | 0.66 |
| 17 | 0.91 | Stavudine | 0.75 |
| 18 | 12.5 | Entecavir | 1.45 |
| 19 | >12.5 | Tenofovir | 2.7 |
| 20 | 0.011 | Adefovir | >6.25 |
| 21 | 0.043 | Apricitabine | 6.34 |
| 22 | 23.4 | Abacavir sulfate | 17.1 |
| 23 | >50 | Efavirenz | >50 |
| 24 | 0.010 | Nevirapine | >50 |
| 25 | 0.0036 | Tenofovir Alafenamide | 0.01 |
| | | (TAF) | |
| 26 | 2.05 | | |
| 27 | 0.0026 | | |

### Example 2

### Cancer Cell Proliferation Activity of Compound 13

A dose response relationship on cell proliferation was assessed for Compound 13 on 140 tumor cell lines. Briefly, compound treatment of cells started one day after seeding with a final DMSO concentration of 0.100, and was performed by nanodrop-dispensing using a Tecan Dispenser. 0.10% DMSO (solvent) and Staurosporine (10 μM) served as high control (10000 viability) and low control (000 viability), respectively. Compound 13 was tested at 10, 3, 1, 0.3, 0.1, 0.03, 0.01, and 0.003 μM.

Cells were cultured in the appropriate media. For the assays, cells were seeded in white cell culture-treated flat and clear bottom multiwell plates and incubated at 37° C. overnight before compound was added. After incubation for 72 h at 37° C. at 5% or 10% CO₂ dependent on the medium, cell plates were equilibrated to room temperature for one hour, CellTiterGlo reagent (Promega) was added and luminescence was measured approximately an hour later using a luminometer.

Raw data were converted into percent cell viability relative to the high and low control, which were set to 10000 and 000, respectively. IC₅₀ calculation was performed using GraphPad Prism software with a variable slope sigmoidal response fitting model using 3% viability as bottom constraint and 100 S viability as top constraint.

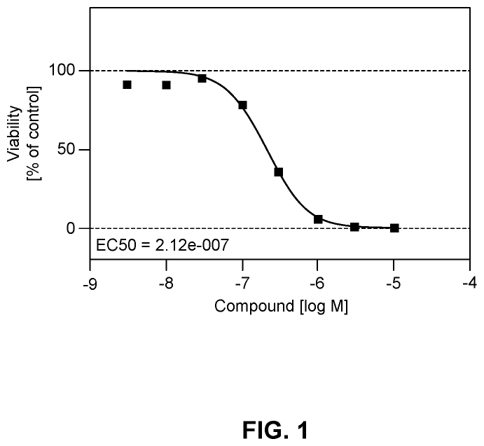

The IC₅₀ values are summarized in Table 6. A representative dose response curve in MV4-11 cells is provided in FIG. 1.

TABLE 6

| | | Cancer Cell | IC₅₀ |
|---|---|---|---|
| No. | Entity | Line | (μM) |
| 1 | Kidney | 786-O | 5.81 |
| 2 | Brain | A172 | >10 |
| 3 | Skin | A2058 | >10 |
| 4 | Ovary | A2780 | 4.58 |
| 5 | Skin | A375 | 2.37 |
| 6 | Lung | A427 | 0.55 |
| 7 | Skin | A431 | 5.06 |
| 8 | Kidney | A498 | >10 |
| 9 | Lung | A549 | >10 |
| 10 | Pancreas | AsPC-1 | >10 |
| 11 | Lung | BEN | >10 |
| 12 | Breast | BT-20 | >10 |
| 13 | Pancreas | BxPC-3 | 5.43 |
| 14 | Kidney | Caki-1 | 9.76 |
| 15 | Kidney | Caki-2 | >10 |
| 16 | Lung | Calu-6 | >10 |
| 17 | Colon | Colo 205 | 1.50 |
| 18 | Lung | COR-L279 | >10 |
| 19 | Ovary | COV434 | 4.41 |
| 20 | Head/Neck | Detroit562 | 4.79 |
| 21 | Colon | DLD-1 | >10 |
| 22 | Prostate | DU-145 | 5.73 |
| 23 | Lung | DV90 | 1.59 |
| 24 | Breast | EFM-19 | 2.65 |
| 25 | Breast | EFM-192A | >10 |
| 26 | Ovary | EFO-27 | 9.90 |
| 27 | Lung | EPLC-272H | 6.63 |
| 28 | Lung | H1299 | 7.68 |
| 29 | Lung | H2228 | >10 |
| 30 | Brain | H4 | >10 |
| 31 | Lung | H460 | 5.68 |
| 32 | Breast | HCC 1569 | >10 |
| 33 | Breast | HCC202 | >10 |
| 34 | Breast | HCC38 | 6.37 |
| 35 | Lung | HCC827 | 4.47 |
| 36 | Colon | HCT116 | 5.84 |
| 37 | Colon | HCT-15 | >10 |
| 38 | Endometrium | HEC-1-A | >10 |
| 39 | Endometrium | HEC-1-B | >10 |
| 40 | Ovary | HeLa | 1.30 |
| 41 | Liver | Hep3B2.1-7 | 0.82 |
| 42 | Blood | HL-60 | 1.13 |
| 43 | Stomach | Hs 746T | 5.39 |
| 44 | Fibrosarcoma | HT-1080 | 7.16 |
| 45 | Colon | HT-29 | >10 |
| 46 | Liver | HuH7 | 1.24 |
| 47 | Duodenum | Hutu 80 | 1.44 |
| 48 | Endometrium | Ishikawa | 6.07 |
| 49 | Bladder | J82 | 5.73 |
| 50 | Breast | JIMT-1 | 7.00 |
| 51 | Blood | Jurkat | 1.17 |
| 52 | Blood | JVM-3 | 2.45 |
| 53 | Blood | K562 | >10 |
| 54 | Blood | KARPAS | 4.07 |
| | | 299 | |
| 55 | Stomach | Kato III | 4.86 |
| 56 | Blood | KG-1 | >10 |
| 57 | Blood | KG-1 a | 5.86 |
| 58 | Blood | KMS-12-BM | 0.96 |
| 59 | Brain | LN229 | 2.23 |
| 60 | Prostate | LnCap | >10 |
| 61 | Lung | LOU-NH91 | >10 |
| 62 | Colon | LOVO | 8.21 |
| 63 | Blood | LP-1 | 3.15 |
| 64 | Blood | M07e | 4.07 |
| 65 | Ovary | MCAS | >10 |
| 66 | Breast | MCF-7 | >10 |
| 67 | Breast | MDA MB | 3.85 |
| | | 231 | |
| 68 | Skin | MDA MB | >10 |
| | | 435 | |
| 69 | Breast | MDA-MB- | >10 |
| | | 468 | |
| 70 | Blood | MEC-1 | 9.85 |
| 71 | Pancreas | Mia PaCA 2 | >10 |
| 72 | Stomach | MKN-1 | 8.76 |
| 73 | Stomach | MKN-45 | >10 |
| 74 | Blood | MOLM-13 | 0.46 |
| 75 | Blood | MOLT-4 | 0.51 |
| 76 | Blood | MV4-11 | 0.21 |
| 77 | Blood | NALM-6 | 0.25 |
| 78 | Lung | NCI-H1048 | 2.49 |
| 79 | Lung | NCI-H1437 | >10 |
| 80 | Lung | NCI-H1563 | >10 |
| 81 | Lung | NCI-H1573 | 8.59 |
| 82 | Lung | NCI-H1581 | 2.97 |
| 83 | Lung | NCI-H1703 | 9.16 |
| 84 | Lung | NCI-H1838 | 7.53 |
| 85 | Lung | NCI-H2009 | 1.23 |
| 86 | Lung | NCI-H2110 | 3.41 |
| 87 | Lung | NCI-H2286 | 5.69 |
| 88 | Lung | NCI-H292 | 8.56 |
| 89 | Lung | NCI-H441 | 3.64 |
| 90 | Lung | NCI-H82 | 6.46 |
| 91 | Lung | NCI-H838 | >10 |
| 92 | Stomach | NCI-N87 | 3.60 |
| 93 | Blood | OCI-AML3 | 7.15 |
| 94 | Blood | OCI-AML5 | 1.37 |
| 95 | Blood | OCI-LY19 | 0.56 |
| 96 | Blood | OPM-2 | >10 |
| 97 | Ovary | OV56 | 1.99 |
| 98 | Ovary | OVCAR-3 | 1.16 |
| 99 | Ovary | OVK18 | 2.21 |
| 100 | Blood | P31/FUJ | 0.40 |
| 101 | Pancreas | PANC-1 | >10 |
| 102 | Prostate | PC3 | 4.01 |
| 103 | Lung | PC-9 | >10 |
| 104 | Lung | RERF-LC- | 2.58 |
| | | Ad2 | |
| 105 | Lung | RERF-LC- | >10 |
| | | MS | |
| 106 | Colon | RKO | 4.51 |
| 107 | Endometrium | RL95-2 | 5.38 |
| 108 | Blood | RPMI 8226 | >10 |
| 109 | Bone | Saos-2 EC | 3.99 |
| 110 | Stomach | SCH | 0.29 |
| 111 | Lung | SCLC-21H | 6.78 |
| 112 | Ovary | SiHa | 2.45 |
| 113 | Bone | SJSA-1 | 6.44 |
| 114 | Breast | SK-BR-3 | >10 |
| 115 | Bone | SK-ES-1 | 4.99 |
| 116 | Lung | SK-LU-1 | >10 |
| 117 | Skin | SK-MEL-3 | >10 |
| 118 | Brain | SK-N-FI | >10 |
| 119 | Brain | SK-N-MC | 3.19 |
| 120 | Brain | SK-N-SH | 6.47 |
| 121 | Ovary | SK-OV3 | 4.00 |
| 122 | Stomach | SNU-1 | 2.89 |
| 123 | Stomach | SNU-16 | 1.95 |
| 124 | Stomach | SNU-216 | 1.74 |
| 125 | Ovary | SNU840 | 4.23 |
| 126 | Blood | SU-DHL-5 | 3.74 |
| 127 | Brain | SW-1783 | 2.25 |
| 128 | Colon | SW480 | >10 |
| 129 | Colon | SW620 | 7.21 |
| 130 | Colon | SW948 | 5.02 |
| 131 | Breast | T-47D | >10 |
| 132 | Colon | T84 | >10 |
| 133 | Brain | T98G | >10 |
| 134 | Brain | U118MG | 5.74 |
| 135 | Brain | U251MG | >10 |
| 136 | Blood | U-266 | >10 |
| 137 | Bone | U2OS | 2.18 |
| 138 | Brain | U87MG | 6.39 |
| 139 | Blood | U-937 | 1.12 |
| 140 | Breast | ZR-75-1 | >10 |

### Example 3

### Cancer Cell Proliferation Activity of Compounds

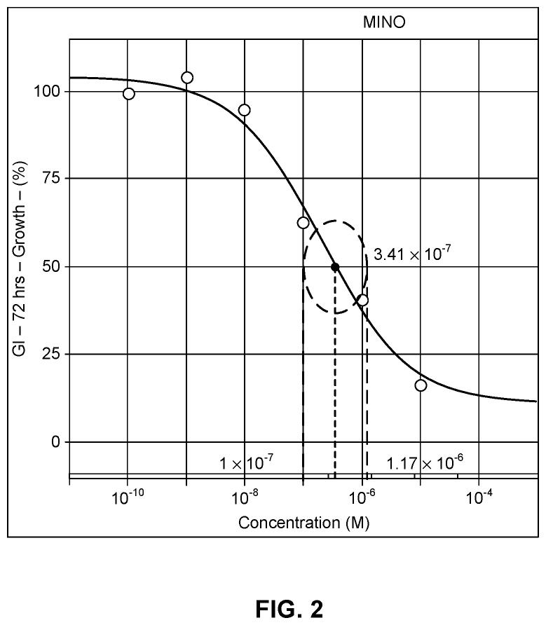

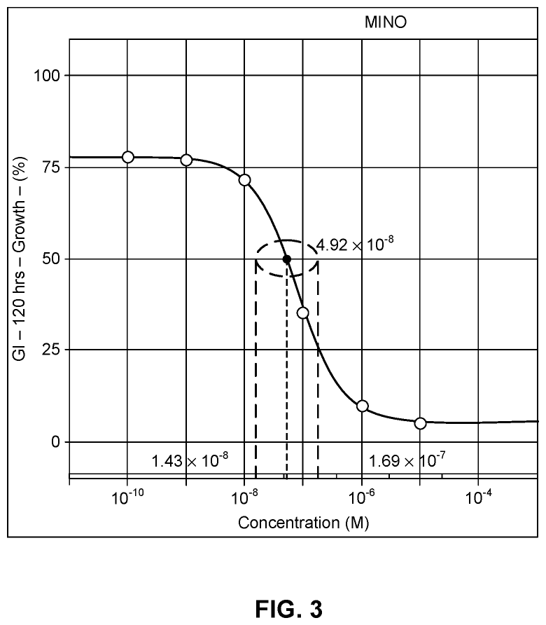

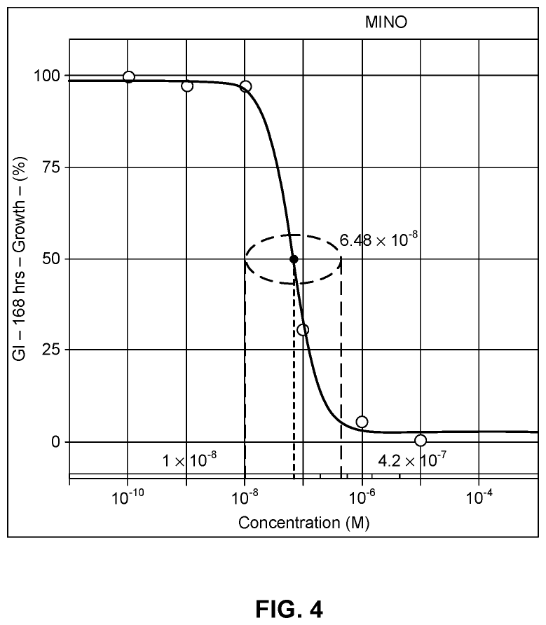

A dose response relationship on cell proliferation is assessed for Compounds 2, 6, 7, 9, 12, 15, 20, 21, 24, 28-30, zalcitabine, tenofovir alafenamide, and islatravir on 95 tumor cell lines after incubation for 168 h using the same basic protocol as described in EXAMPLE 2. The IC₅₀ values for Compound 9 are summarized in Table 7. Representative dose response curves in MINO cells at 72 h, 120 h, and 168 h for Compound 9 are provided in FIGS. 2-4.

TABLE 7

| | | IC₅₀ (μM) | | |
|---|---|---|---|---|
| Number | Cancer Cell Line | 72 h | 120 h | 168 h |
| 1 | 22RV1 | 3.80 | 3.28 | 3.69 |
| 2 | 5637 | >10 | >10 | >10 |
| 3 | 786O | >10 | >10 | >10 |
| 4 | A204 | >10 | >10 | >10 |
| 5 | A2780 | >10 | >10 | >10 |
| 6 | A375 | >10 | >10 | >10 |
| 7 | A431 | >10 | 3.18 | 1.42 |
| 8 | A549 | >10 | >10 | >10 |
| 9 | A673 | >10 | >10 | >10 |
| 10 | ACHN | >10 | 0.91 | 0.94 |
| 11 | ASPC1 | >10 | 0.72 | 0.49 |
| 12 | BT20 | >10 | >10 | >10 |
| 13 | BXPC3 | >10 | 1.29 | 0.59 |
| 14 | C33A | >10 | 2.93 | 3.07 |
| 15 | CACO2 | >10 | >10 | >10 |
| 16 | CAKI1 | >10 | >10 | >10 |
| 17 | CALU6 | >10 | >10 | >10 |
| 18 | CASKI | >10 | >10 | >10 |
| 19 | CLS439 | >10 | >10 | >10 |
| 20 | COLO205 | >10 | 1.15 | 0.87 |
| 21 | COLO678 | >10 | >10 | >10 |
| 22 | DLD1 | >10 | >10 | >10 |
| 23 | DU145 | >10 | >10 | >10 |
| 24 | EFO21 | >10 | >10 | >10 |
| 25 | EJ28 | >10 | >10 | >10 |
| 26 | GRANTA-519 | 4.98 | 0.24 | 0.35 |
| 27 | HCT116 | >10 | >10 | >10 |
| 28 | HCT15 | >10 | >10 | >10 |
| 29 | HEK293 | >10 | >10 | >10 |
| 30 | HELA | >10 | >10 | >10 |
| 31 | HEPG2 | >10 | >10 | >10 |
| 32 | HL-60 | >10 | >10 | >10 |
| 33 | HS578T | >10 | >10 | >10 |
| 34 | HS729 | >10 | >10 | >10 |
| 35 | HT1080 | >10 | >10 | >10 |
| 36 | HT29 | >10 | >10 | >10 |
| 37 | IGROV1 | >10 | >10 | >10 |
| 38 | IMR90 | >10 | >10 | >10 |
| 39 | J82 | >10 | >10 | >10 |
| 40 | JAR | >10 | >10 | >10 |
| 41 | JEG3 | >10 | >10 | >10 |
| 42 | JIMT1 | >10 | >10 | >10 |
| 43 | K-562 | >10 | >10 | >10 |
| 44 | KASUMI | >10 | >10 | >10 |
| 45 | L363 | >10 | >10 | >10 |
| 46 | LOVO | >10 | 3.28 | 2.06 |
| 47 | MCF7 | >10 | >10 | >10 |
| 48 | MDAMB231 | >10 | >10 | >10 |
| 49 | MDAMB435 | >10 | >10 | >10 |
| 50 | MDAMB436 | >10 | >10 | >10 |
| 51 | MDAMB468 | >10 | >10 | >10 |
| 52 | MG63 | >10 | >10 | >10 |
| 53 | MHHES1 | >10 | >10 | >10 |
| 54 | MIAPACA2 | >10 | >10 | >10 |
| 55 | MINO | 0.34 | 0.049 | 0.065 |
| 56 | MT3 | >10 | 3.04 | >10 |
| 57 | MV4-11 | 2.48 | 0.251 | 1.72 |
| 58 | NCI-H23 | >10 | >10 | >10 |
| 59 | NCI-H292 | >10 | >10 | >10 |
| 60 | NCIH358M | >10 | >10 | 0.67 |
| 61 | NCIH460 | >10 | >10 | >10 |
| 62 | NCIH82 | >10 | 9.1 | >10 |
| 63 | OVCAR3 | >10 | >10 | >10 |
| 64 | OVCAR4 | >10 | >10 | >10 |
| 65 | PANC1 | >10 | >10 | >10 |
| 66 | PANC1005 | >10 | >10 | >10 |
| 67 | PBMC | >10 | >10 | >10 |
| 68 | PC3 | >10 | >10 | >10 |
| 69 | PLCPRF5 | >10 | >10 | >10 |
| 70 | RAMOS | 4.04 | 0.004 | 1.18 |
| 71 | RD | >10 | >10 | >10 |
| 72 | RDES | >10 | >10 | >10 |
| 73 | SAOS2 | >10 | >10 | >10 |
| 74 | SF268 | >10 | >10 | >10 |
| 75 | SF295 | >10 | >10 | >10 |
| 76 | SKBR3 | 1.45 | 0.96 | 0.37 |
| 77 | SKHEP1 | >10 | >10 | >10 |
| 78 | SKLMS1 | >10 | >10 | >10 |
| 79 | SKMEL28 | >10 | >10 | >10 |
| 80 | SKMEL5 | >10 | >10 | >10 |
| 81 | SKNAS | >10 | >10 | >10 |
| 82 | SKNSH | >10 | >10 | >10 |
| 83 | SKOV3 | >10 | >10 | >10 |
| 84 | SNB75 | >10 | >10 | >10 |
| 85 | SU-DHL-10 | 0.14 | 61.5 | 3.86 |
| 86 | SU-DHL-6 | >10 | 4.99 | 1.3 |
| 87 | SW620 | >10 | >10 | >10 |
| 88 | T24 | >10 | >10 | >10 |
| 89 | TE671 | >10 | >10 | >10 |
| 90 | THP-1 | >10 | >10 | >10 |
| 91 | U2OS | >10 | >10 | >10 |
| 92 | U87MG | >10 | >10 | >10 |
| 93 | UMUC3 | >10 | >10 | >10 |
| 94 | UO31 | >10 | >10 | >10 |
| 95 | WSU-NHL | 3.01 | 0.42 | 0.87 |

### Example 4

### Synthesis of 4-amino-1-((2R,4S,5R)-5-ethynyl-4-hydroxy-5 (hydroxymethyl)tetrahydrofuran-2-yl)pyrimidin-2(1H)-one (Compound 7)

### Step 1: Synthesis of (2R,3S,5R)-5-(4-benzamido-2-oxopyrimidin-1(2H)-yl)-2-ethynyl-2-(((4-methylbenzoyl)oxy)methyl)tetrahydrofuran-3-yl 4-methylbenzoate and (2R,3S,5S)-5-(4-benzamido-2-oxopyrimidin-1(2H)-yl)-2-ethynyl-2-(((4-methylbenzoyl) oxy)methyl)tetrahydrofuran-3-yl 4-methylbenzoate

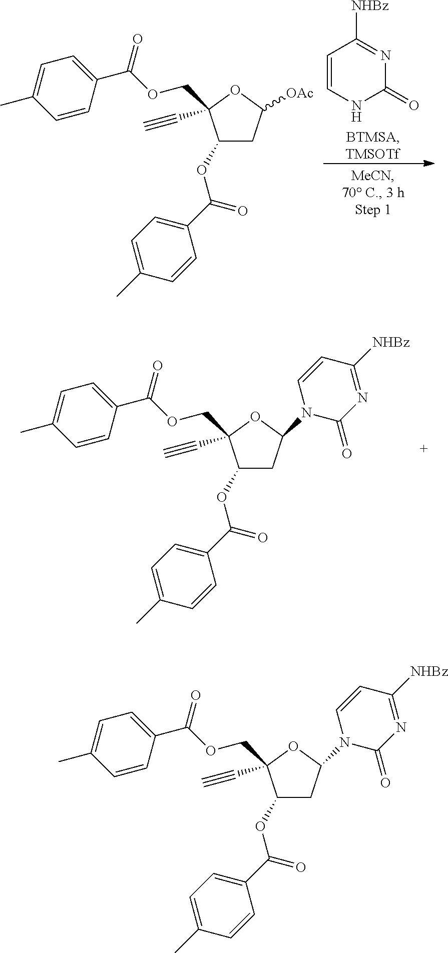

To a solution of N-(2-oxo-1H-pyrimidin-4-yl)benzamide (118 mg, 0.55 mmol) in MeCN (20 mL) was added BTMSA (234 mg, 1.37 mmol) at room temperature. The resulting mixture was heated at 70° C. for 1 h. After cooling to room temperature, TMSOTf (122 mg, 0.55 mmol) was added and the mixture was reheated to 70° C., then a solution of [(2R,3S)-5-acetoxy-2-ethynyl-3-(4-methylbenzoyl)oxy-tetrahydrofuran-2-yl]methyl4-methylbenzoate (200 mg, 0.46 mmol) in MeCN (5 mL) was added dropwise. After stirring at 70° C. for 2 h, the reaction mixture was poured into water (50 mL) and extracted with EtOAc (50 mL×2). The layers were separated, and the organic layer was concentrated. The residue was purified by prep-TLC eluting with 50% EtOAc in petroleum ether to give [(2R,3S,5R)-5-(4-benzamido-2-oxo-pyrimidin-1-yl)-2-ethynyl-3-(4-methylbenzoyl)oxy-tetrahydrofuran-2-yl]methyl 4-methylbenzoate (R_{f}=0.5) (60 mg, 22% yield) as a white solid and [(2R,3S,5S)-5-(4-benzamido-2-oxo-pyrimidin-1-yl)-2-ethynyl-3-(4-methyl benzoyl)oxy-tetrahydrofuran-2-yl]methyl 4-methylbenzoate (R_{f}=0.4) (60 mg, 22% yield) as a white solid.

### Step 2: Synthesis of 4-amino-1-((2R,4S,5R)-5-ethynyl-4-hydroxy-5 (hydroxymethyl)tetrahydrofuran-2-yl)pyrimidin-2(1H)-one

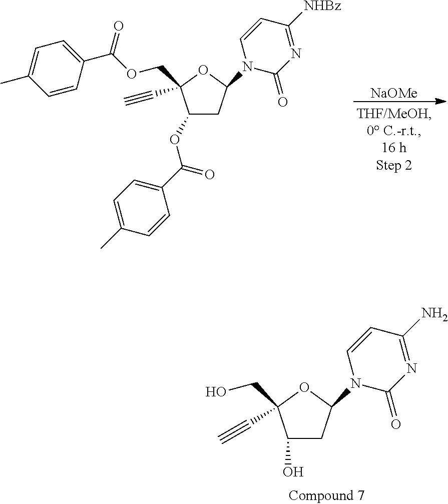

To a mixture of [(2R,3S,5R)-5-(4-benzamido-2-oxo-pyrimidin-1-yl)-2-ethynyl-3-(4-methylbenzoyl)oxy-tetrahydrofuran-2-yl]methyl4-methylbenzoate (60 mg, 0.1 mmol) in THE (5 mL) was added dropwise a solution of NaOMe (7 mg, 0.13 mmol) in MeOH (2 mL) at 0° C., then the resulting mixture was stirred at room temperature for 16 h. After that, the reaction mixture was concentrated under reduced pressure. The residue was purified by prep-HPLC to afford Compound 7 (8.8 mg, 34% yield) as a white solid. ¹H NMR (400 MHz, DMSO-d6): δ 7.77 (d, J=7.2 Hz, 1H), 7.17-7.11 (m, 2H), 6.15-6.12 (m, 1H), 5.71 (d, J=7.2 Hz, 1H), 5.46 (s, 1H), 5.39 (s, 1H), 4.30-4.29 (m, 1H), 3.60-3.50 (m, 2H), 3.48 (s, 1H), 2.26-2.20 (m, 1H), 2.10-2.01 (m, 1H). LCMS (ESI): m/z 252.2 (M+H)⁺.

### Example 5

### Synthesis of (2R,3S,5R)-5-(6-amino-2-fluoro-9H-purin-9-yl)-2-(hydroxymethyl)-2-vinyltetrahydrofuran-3-ol (Compound 20)

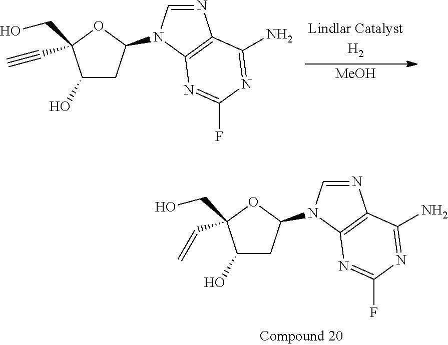 [SMILES: "MeOH" ; C#C[C@]1(CO)O[C@@H](n2cnc3c(N)nc(F)nc32)C[C@@H]1O ; C=C[C@]1(CO)O[C@@H](n2cnc3c(N)nc(F)nc32)C[C@@H]1O]

(2R,3S,5R)-5-(6-amino-2-fluoro-9H-purin-9-yl)-2-ethynyl-2-(hydroxymethyl)tetrahydrofuran-3-ol (79.3 mg, 270 μmol) and Lindlar Catalyst (10.0 mg, 270 μmol) were solubilized in MeOH (5.00 mL) at room temperature. Nitrogen atmosphere was bubbled through the solution for 10 min and then hydrogen was bubbled through the solution for 1 h using a balloon. The reaction was sealed and stirred for 18 h at room temperature. Then, nitrogen atmosphere was bubbled through the solution for 5 min, then the resulting mixture was filtered over a Celite® pad and it was rinsed with MeOH (15 mL). The filtrate was concentrated. The desired product was purified by prep-HPLC using a XBridge Prep C18, 5 μm 19×10 mm pre-column, CSH Prep C18 OBD, 5 μm, 30×75 mm column with MeOH (Eluent B) and AmF pH 3.8 (Eluent A) using an isocratic at 5% B for 1 min pre-run and a gradient of 5% B isocratic for 1 min, 5% B to 25% B for 11 minutes, 25% B to 100% B for 0.1 minute, hold 100% B for 2.9 minutes with a 45 mL/min flowrate and a 15 min runtime, affording (2R,3S,5R)-5-(6-amino-2-fluoro-9H-purin-9-yl)-2-(hydroxymethyl)-2-vinyltetrahydrofuran-3-ol (42.4 mg, 59%). LC-MS (ESI) m/z calcd for C₁₂H₁₄FN₅O₃: 295.1. Found 296.2 [M+H]⁺. ¹H NMR (400 MHz, DMSO-d6): δ 8.36 (s, 1H), 7.82 (br s, 1H), 6.24-6.22 (m, 1H), 5.99-5.92 (m, 1H), 5.41-5.36 (m, 1H), 5.31-5.30 (m, 1H), 5.23-5.20 (m, 1H), 5.11 (m, 1H), 4.64 (q, J=6.0 Hz, 1H), 3.52-3.48 (m, 2H), 2.60-2.57 (m, 1H), 2.29-2.22 (m, 1H).

### Example 6

### Synthesis of 4-amino-1-((2R,4S,5R)-5-ethyl-4-hydroxy-5-(hydroxymethyl)tetrahydrofuran-2-yl)pyrimidin-2(1H)-one (Compound 21)

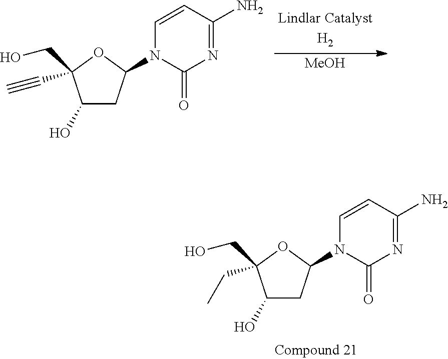 [SMILES: "MeOH" ; C#C[C@]1(CO)O[C@@H](n2ccc(N)nc2=O)C[C@@H]1O ; CC[C@]1(CO)O[C@@H](n2ccc(N)nc2=O)C[C@@H]1O]

4-amino-1-((2R,4S,5R)-5-ethynyl-4-hydroxy-5-(hydroxymethyl)tetrahydrofuran-2-yl)pyrimidin-2(1H)-one (7.20 mg, 28.7 μmol) was dissolved in MeOH in a 5-mL vial with a rubber septum. Then solid Lindlar Catalyst (7.20 mg, 28.7 μmol) was added and the reaction mixture was flushed with H2 balloon for 30 min. This reaction was stirred till full conversion to the title compound was observed by LC-MS. Then the reaction mixture was filtered through Celite® and washed with MeOH and the solvent was removed under reduced pressure, affording 4-amino-1-((2R,4S,5R)-5-ethyl-4-hydroxy-5-(hydroxymethyl)tetrahydrofuran-2-yl)pyrimidin-2(1H)-one (2.85 mg, 39%) as an off-white powder. LC-MS (ESI) m/z calcd for C₁₁H₁₆N₃O₄: 254.12. Found 254.4 [M−H]−. 1H-NMR (400 MHz, CD3OD) δ 8.05 (d, J=7.6 Hz, 1H), 6.21-6.06 (m, 1H), 5.95-5.77 (d, J=5.8 Hz, 1H), 4.41-4.33 (m, 1H), 3.73-3.60 (m, 1H), 3.60-3.47 (m, 1H), 2.52-2.11 (m, 1H), 1.85-1.47 (m, 1H), 1.04-0.84 (m, 5H).

### Example 7

### Cancer Cell Proliferation Activity

Dose response relationships on cell proliferation was assessed for representative nucleosides on cancer cell lines. Briefly, compound treatment of cells started one day after seeding with a final DMSO concentration of 0.1%, and was performed by nanodrop-dispensing using a Tecan Dispenser. 0.1% DMSO (solvent) and Staurosporine (10 μM) served as high control (100% viability) and low control (0% viability), respectively. Cells were cultured in the appropriate media. For the assays, cells were seeded in white cell culture-treated flat and clear bottom multiwell plates and incubated at 37° C. overnight before compound was added. After incubation for 72 h at 37° C. at 5% or 10% CO2 dependent on the medium, cell plates were equilibrated to room temperature for one hour, CellTiterGlo reagent (Promega) was added and luminescence was measured approximately an hour later using a luminometer.

Raw data were converted into percent cell viability relative to the high and low control, which were set to 100% and 0%, respectively. IC₅₀ calculation was performed using GraphPad Prism software with a variable slope sigmoidal response fitting model using 0% viability as bottom constraint and 100% viability as top constraint.

The IC₅₀ values are summarized in Table 8 and Table 9.

TABLE 8

| Cpd. No. | | 13 | 15 | 7 | 29 | 30 | 21 | 25 | 9 |
|---|---|---|---|---|---|---|---|---|---|
| Cell Line | Cancer Type | IC₅₀ (μM) | | | | | | | |
| 5637 | bladder | 2.27 | 0.37 | 1.24 | 0.23 | 0.18 | >50 | 3.52 | 0.81 |
| 22RV1 | prostate | 2.22 | 3.69 | 4.80 | 1.04 | 1.35 | >50 | >50 | 0.55 |
| 786O | kidney | >50 | >50 | 4.28 | 1.61 | 0.38 | >50 | >50 | 3.54 |
| A204 | muscle | 0.27 | >50 | 0.18 | 0.03 | 0.02 | 0.27 | 1.58 | 9.82 |
| A2780 | ovary | 15.61 | >50 | 2.72 | 0.64 | 0.25 | >50 | >50 | 4.18 |
| A375 | skin | 2.35 | >50 | 3.54 | 0.57 | 0.33 | >50 | 2.34 | >50 |
| A431 | skin | 3.03 | 1.42 | 2.01 | 0.30 | 0.28 | >50 | >50 | 0.11 |
| A549 | lung | 2.06 | >50 | 2.19 | 0.88 | 0.32 | 5.54 | 3.25 | 1.24 |
| A673 | bone | >50 | >50 | 1.97 | 0.20 | 0.50 | >50 | >50 | 1.96 |
| ACHN | kidney | 1.44 | 0.94 | 2.76 | 0.71 | 0.34 | 2.74 | 5.89 | 1.05 |
| ASPC1 | pancreas | 1.33 | 0.49 | 1.23 | 0.28 | 0.13 | >50 | 5.31 | 2.58 |
| BT20 | breast | >50 | >50 | >50 | 1.47 | 0.67 | >50 | >50 | >50 |
| BXPC3 | pancreas | 1.43 | 0.59 | 4.12 | 0.35 | 0.13 | >50 | 3.54 | 0.23 |
| C33A | endometrial | 2.03 | 3.07 | 1.03 | 0.24 | 0.19 | 6.53 | 3.70 | 4.56 |
| CACO2 | colon | >50 | >50 | >50 | >50 | 2.96 | >50 | >50 | >50 |
| CAKI1 | kidney | 1.84 | >50 | 0.45 | 0.11 | 0.07 | 1.49 | 5.33 | >50 |
| CALU6 | lung | >50 | >50 | 6.33 | 2.54 | 0.50 | >50 | 6.13 | >50 |
| CASKI | endometrial | >50 | >50 | >50 | 5.22 | 1.28 | >50 | >50 | 11.56 |
| CLS439 | bladder | >50 | >50 | >50 | 3.00 | 2.65 | >50 | >50 | >50 |
| COLO205 | colon | 1.47 | 0.87 | 0.29 | 0.16 | 0.16 | 3.44 | 1.80 | 0.91 |
| COLO678 | colon | 4.09 | >50 | >50 | 10.79 | 1.71 | >50 | >50 | >50 |
| DLD1 | colon | >50 | >50 | 4.63 | 0.77 | 0.58 | >50 | >50 | 4.35 |
| DU145 | prostate | 4.07 | >50 | 3.40 | 0.33 | 0.28 | >50 | 6.33 | 2.42 |
| EFO21 | ovary | 4.00 | >50 | >50 | 1.19 | 0.45 | >50 | >50 | 2.41 |
| EJ28 | bladder | 6.82 | >50 | 3.05 | 2.34 | 1.02 | >50 | >50 | 2.21 |
| GRANTA-519 | hematological | 2.44 | 0.35 | 0.33 | 0.08 | 0.08 | 1.61 | 0.89 | 3.24 |
| HCT116 | colon | 3.37 | >50 | 2.44 | 0.55 | 0.31 | 3.01 | 3.20 | >50 |
| HCT15 | colon | >50 | >50 | 3.28 | 0.81 | 0.54 | 9.11 | >50 | 4.08 |
| HEK293 | kidney | 7.69 | >50 | >50 | 12.70 | 10.90 | >50 | 6.09 | 7.63 |
| HELA | endometrial | 1.20 | >50 | 2.11 | 0.75 | 0.33 | >50 | 3.10 | >50 |
| HEPG2 | liver | 1.24 | >50 | 3.08 | 2.67 | 1.30 | >50 | >50 | 1.60 |
| HL-60 | hematological | 2.06 | >50 | 2.03 | 0.54 | 0.36 | >50 | 3.46 | 4.04 |
| HS578T | breast | >50 | >50 | 4.73 | 1.79 | 0.92 | >50 | >50 | >50 |
| HS729 | muscle | 1.51 | >50 | 2.10 | 0.55 | 0.37 | >50 | 3.42 | >50 |
| HT1080 | connective tissue | 3.88 | >50 | 1.58 | 0.35 | 0.27 | >50 | 3.72 | 2.88 |
| HT29 | colon | 2.28 | >50 | 4.06 | 1.06 | 0.41 | >50 | >50 | 1.47 |
| IGROV1 | ovary | 2.76 | >50 | 3.42 | 1.37 | 0.52 | >50 | >50 | 5.01 |
| IMR90 | lung | >50 | >50 | >50 | 2.73 | 2.12 | >50 | 3.47 | >50 |
| J82 | bladder | 1.97 | >50 | 2.77 | 0.84 | 0.64 | >50 | >50 | 1.16 |
| JAR | placenta | >50 | >50 | >50 | >50 | 5.24 | >50 | >50 | 7.25 |
| JEG3 | placenta | >50 | >50 | >50 | 1.90 | 1.25 | >50 | >50 | 0.99 |
| JIMT1 | breast | 2.04 | >50 | 0.82 | 0.21 | 0.08 | >50 | 5.55 | 4.47 |
| K-562 | hematological | >50 | >50 | 3.52 | 1.61 | 1.44 | >50 | >50 | 4.38 |
| KASUMI-1 | hematological | 0.10 | >50 | 0.32 | 0.22 | 0.08 | >50 | 0.18 | >50 |
| L-363 | hematological | >50 | >50 | >50 | >50 | >50 | >50 | 3.29 | 1.96 |
| LOVO | colon | 3.10 | 2.06 | 1.90 | 0.44 | 0.34 | >50 | >50 | 1.62 |
| MCF7 | breast | 5.95 | >50 | >50 | >50 | 1.96 | >50 | 5.68 | 1.19 |
| MDAMB231 | breast | 1.32 | >50 | 0.48 | 0.13 | 0.05 | 2.85 | 2.85 | >50 |
| MDAMB435 | skin | >50 | >50 | 3.49 | 2.12 | 0.82 | >50 | >50 | 4.39 |
| MDAMB436 | breast | 1.50 | >50 | 3.80 | 0.36 | 0.31 | >50 | 5.23 | 1.37 |
| MDAMB468 | breast | >50 | >50 | >50 | 1.20 | 0.29 | >50 | >50 | >50 |
| MG63 | bone | >50 | >50 | 2.93 | 0.57 | 0.38 | >50 | 4.11 | 2.44 |
| MHHES1 | bone | >50 | >50 | 2.06 | 0.21 | 0.72 | >50 | 3.92 | 1.53 |
| MIAPACA2 | pancreas | 6.24 | >50 | 5.54 | 1.93 | 0.54 | >50 | >50 | 3.75 |
| MINO | hematological | 0.46 | 0.06 | 0.20 | 0.07 | 0.08 | 1.30 | 1.51 | 3.74 |
| MT3 | breast | 0.37 | >50 | 0.57 | 0.12 | 0.05 | 3.29 | 1.62 | 1.46 |
| MV4-11 | hematological | 1.02 | 1.72 | 4.32 | 0.60 | 0.57 | 4.39 | 2.25 | 0.39 |
| NCI-H23 | lung | 4.67 | >50 | 2.79 | 1.34 | 0.33 | >50 | 1.68 | 2.42 |
| NCIH292 | lung | 1.07 | >50 | 1.94 | 0.79 | 0.28 | 4.62 | 5.22 | >50 |
| NCIH358M | lung | 0.33 | 0.67 | 0.29 | 0.04 | 0.05 | 1.49 | 1.63 | >50 |
| NCIH460 | ung | 1.41 | >50 | 2.99 | 2.05 | 0.42 | 4.01 | >50 | 1.06 |
| NCIH82 | lung | 15.40 | >50 | 17.05 | >50 | 2.53 | >50 | >50 | 2.35 |
| OVCAR3 | ovary | 0.76 | >50 | 2.39 | 0.58 | 0.38 | >50 | 1.82 | 5.60 |
| OVCAR4 | ovary | >50 | >50 | >50 | 4.18 | 1.05 | >50 | 3.27 | >50 |
| PANC1 | pancreas | >50 | >50 | >50 | 3.43 | 1.15 | >50 | >50 | 6.40 |
| PANC1005 | pancreas | 3.08 | >50 | 2.57 | 0.65 | 0.43 | >50 | 6.31 | 16.40 |
| PBMC | hematological | 0.19 | >50 | >50 | 0.35 | 1.03 | 11.78 | >50 | >50 |
| PC3 | prostate | 1.80 | >50 | 1.09 | 0.20 | 0.15 | >50 | 3.02 | 6.33 |
| PLCPRF5 | liver | 2.10 | >50 | >50 | >50 | 2.16 | >50 | >50 | 3.24 |
| RAMOS | hematological | 1.17 | 1.18 | 0.37 | 0.17 | 0.11 | 1.72 | 1.85 | 5.30 |
| RD | muscle | >50 | >50 | 3.86 | 1.40 | 0.79 | >50 | 3.39 | 4.22 |
| RDES | bone | 2.48 | >50 | >50 | 1.62 | 2.20 | >50 | >50 | 3.93 |
| SAOS2 | bone | 1.66 | >50 | 0.64 | 0.11 | 0.08 | >50 | 1.73 | 4.72 |
| SF268 | brain | >50 | >50 | 1.71 | 0.35 | 0.15 | >50 | >50 | 1.65 |
| SF295 | brain | 7.16 | >50 | 3.90 | 1.13 | 0.73 | >50 | >50 | 4.49 |
| SKBR3 | breast | 4.61 | 0.37 | 2.11 | 0.65 | 0.19 | >50 | 3.13 | 0.12 |
| SKHEP1 | liver | 2.29 | >50 | 1.29 | 0.24 | 0.12 | >50 | 4.99 | 1.29 |
| SKLMS1 | uterus | 2.38 | >50 | 4.20 | 3.74 | 1.35 | >50 | >50 | 5.61 |
| SKMEL28 | skin | 6.06 | >50 | >50 | 2.63 | 1.18 | >50 | 3.10 | >50 |
| SKMEL5 | skin | >50 | >50 | >50 | >50 | 3.65 | >50 | >50 | 5.52 |
| SKNAS | brain | 2.44 | >50 | >50 | 4.41 | 1.41 | >50 | 0.75 | >50 |
| SKNSH | brain | 2.29 | >50 | >50 | >50 | >50 | >50 | 0.60 | 2.44 |
| SKOV3 | ovary | 2.02 | >50 | 3.11 | 0.89 | 0.28 | >50 | 2.37 | 1.64 |
| SNB75 | brain | 12.84 | >50 | 9.75 | 0.84 | 0.83 | >50 | >50 | >50 |
| SU-DHL-10 | hematological | 5.60 | 3.86 | 0.69 | 0.50 | 0.36 | >50 | 4.83 | 3.15 |
| SU-DHL-6 | hematological | 4.19 | 1.30 | 1.06 | 0.80 | 0.33 | 3.80 | 6.01 | 4.69 |
| SW620 | colon | 1.82 | >50 | 2.93 | 2.64 | 0.65 | >50 | >50 | 0.91 |
| T24 | bladder | 4.54 | >50 | 1.03 | 0.18 | 0.08 | 3.46 | >50 | 3.76 |
| TE671 | muscle | >50 | >50 | 3.49 | 1.75 | 0.44 | >50 | 3.05 | 5.90 |
| THP-1 | hematological | 1.97 | >50 | 3.43 | 0.97 | 1.23 | >50 | 3.23 | 1.12 |
| U2OS | bone | >50 | >50 | >50 | 5.32 | 0.67 | >50 | >50 | >50 |
| U87MG | brain | 2.38 | >50 | 1.30 | 0.50 | 0.33 | >50 | 5.24 | 11.88 |
| UMUC3 | bladder | >50 | >50 | 3.07 | 1.16 | 0.56 | >50 | >50 | 3.39 |
| UO31 | kidney | 3.29 | >50 | 1.53 | 0.66 | 0.15 | 9.45 | >50 | >50 |
| WSU-NHL | hematological | 2.77 | 0.87 | 0.34 | 0.22 | 0.21 | 2.82 | 2.55 | 3.53 |

TABLE 9

| | Cpd. No. | | | |
|---|---|---|---|---|
| Cell Line | 31 | 32 | islatravir | elvucitabine |
| 5637 | >50 | 0.45 | 2.57 | >50 |
| 22RV1 | 1.06 | 1.33 | >50 | >50 |
| 786O | >50 | 1.07 | 3.31 | >50 |
| A204 | 2.55 | 0.02 | 1.46 | 0.41 |
| A2780 | >50 | 0.72 | 4.93 | >50 |
| A375 | >50 | 0.71 | 2.69 | >50 |
| A431 | 2.60 | 0.47 | 2.87 | 17.08 |
| A549 | >50 | 0.44 | 2.98 | >50 |
| A673 | 5.25 | 1.74 | 4.57 | >50 |
| ACHN | 4.02 | 0.39 | 2.48 | 1.87 |
| ASPC1 | >50 | 0.58 | 4.97 | 2.46 |
| BT20 | >50 | 0.58 | 2.79 | >50 |
| BXPC3 | 6.34 | 0.54 | 2.77 | >50 |
| C33A | 1.75 | 0.24 | 2.65 | >50 |
| CACO2 | >50 | >50 | >50 | >50 |
| CAKI1 | >50 | 0.11 | 0.69 | 2.95 |
| CALU6 | >50 | 0.72 | 5.30 | >50 |
| CASKI | >50 | 3.96 | 5.31 | >50 |
| CLS439 | >50 | 2.27 | >50 | >50 |
| COLO205 | >50 | 0.21 | 4.98 | >50 |
| COLO678 | >50 | 3.76 | 3.11 | >50 |
| DLD1 | 4.59 | 2.65 | >50 | >50 |
| DU145 | 5.46 | 0.31 | 2.53 | 1.41 |
| EFO21 | >50 | 0.79 | 4.62 | >50 |
| EJ28 | >50 | 1.53 | 5.71 | >50 |
| GRANTA- | 3.15 | 0.07 | 1.68 | 1.35 |
| 519 | | | | |
| HCT116 | >50 | 0.47 | 4.69 | >50 |
| HCT15 | >50 | 0.46 | 5.90 | >50 |
| HEK293 | >50 | >50 | 4.16 | >50 |
| HELA | >50 | 1.00 | 2.98 | >50 |
| HEPG2 | 5.08 | 1.00 | 3.14 | 6.29 |
| HL-60 | >50 | 1.01 | 3.49 | 3.36 |
| HS578T | >50 | 0.97 | 2.94 | >50 |
| HS729 | >50 | 0.58 | 4.24 | >50 |
| HT1080 | >50 | 0.38 | 3.19 | >50 |
| HT29 | >50 | 1.27 | 4.63 | >50 |
| IGROV1 | >50 | 0.51 | 3.03 | >50 |
| IMR90 | >50 | 1.58 | 2.46 | >50 |
| J82 | 4.88 | 0.76 | 5.43 | >50 |
| JAR | >50 | >50 | 5.41 | >50 |
| JEG3 | >50 | 3.34 | 3.95 | >50 |
| JIMT1 | >50 | 0.19 | 1.58 | 8.56 |
| K-562 | >50 | 4.45 | 4.46 | >50 |
| KASUMI-1 | >50 | 0.27 | 0.96 | 18.70 |
| L-363 | >50 | >50 | 3.21 | >50 |
| LOVO | 3.55 | 0.29 | 2.79 | >50 |
| MCF7 | >50 | 3.17 | >50 | >50 |
| MDAMB231 | >50 | 0.10 | 2.56 | 5.38 |
| MDAMB435 | >50 | 1.30 | >50 | >50 |
| MDAMB436 | 5.24 | 0.95 | 3.59 | >50 |
| MDAMB468 | >50 | 3.11 | 4.00 | >50 |
| MG63 | >50 | 0.48 | >50 | >50 |
| MHHES1 | 3.07 | 1.66 | 3.51 | 1.96 |
| MIAPACA2 | >50 | 1.15 | >50 | >50 |
| MINO | 3.91 | 0.06 | 1.34 | 4.93 |
| MT3 | 3.77 | 0.08 | 2.64 | >50 |
| MV4-11 | 0.38 | 0.84 | 2.39 | >50 |
| NCI-H23 | 4.05 | 0.72 | 2.96 | >50 |
| NCIH292 | >50 | 0.92 | 3.68 | 4.68 |
| NCIH358M | >50 | 0.12 | 0.73 | 4.54 |
| NCIH460 | 2.80 | 0.77 | 3.31 | >50 |
| NCIH82 | 5.25 | 1.15 | 2.73 | >50 |
| OVCAR3 | >50 | 0.64 | 4.15 | >50 |
| OVCAR4 | >50 | 8.05 | 3.10 | >50 |
| PANC1 | >50 | 0.62 | >50 | >50 |
| PANC1005 | >50 | >50 | 3.87 | >50 |
| PBMC | >50 | 0.71 | >50 | >50 |
| PC3 | >50 | 0.18 | 3.12 | >50 |
| PLCPRF5 | 5.05 | >50 | >50 | >50 |
| RAMOS | >50 | 0.24 | 1.88 | 1.96 |
| RD | >50 | 1.03 | 3.30 | >50 |
| RDES | 3.65 | 1.90 | 3.01 | >50 |
| SAOS2 | >50 | 0.11 | 1.65 | >50 |
| SF268 | 14.59 | 0.26 | >50 | >50 |
| SF295 | >50 | 3.25 | 2.91 | >50 |
| SKBR3 | 3.44 | 0.26 | 1.76 | 3.47 |
| SKHEP1 | 3.15 | 0.32 | 2.66 | >50 |
| SKLMS1 | 2.88 | 0.86 | 3.35 | >50 |
| SKMEL28 | >50 | 1.32 | 3.84 | >50 |
| SKMEL5 | >50 | 6.18 | 6.02 | >50 |
| SKNAS | >50 | 1.22 | 2.98 | >50 |
| SKNSH | >50 | >50 | 3.37 | 6.99 |
| SKOV3 | >50 | 1.46 | 3.25 | >50 |
| SNB75 | 4.52 | 0.55 | 3.27 | >50 |
| SU-DHL-10 | >50 | 0.56 | 2.60 | >50 |
| SU-DHL-6 | 3.92 | 0.31 | 1.68 | 4.20 |
| SW620 | >50 | 1.14 | 3.81 | >50 |
| T24 | 3.01 | 0.29 | 2.68 | >50 |
| TE671 | >50 | 1.02 | >50 | >50 |
| THP-1 | 2.49 | 0.65 | 2.93 | 2.04 |
| U2OS | >50 | 2.45 | 2.53 | >50 |
| U87MG | 5.23 | 0.40 | 2.75 | 18.51 |
| UMUC3 | >50 | 1.21 | 3.53 | >50 |
| UO31 | >50 | 0.55 | 4.71 | 11.25 |
| WSU-NHL | 4.26 | 0.15 | 3.17 | 2.46 |

The breadth and scope of the present disclosure should not be limited by any of the above-described exemplary embodiments, but should be defined only in accordance with the following claims and their equivalents.

What is claimed is:

1. A method for treating cancer in patient in need thereof, the method comprising administering a therapeutically effective amount of: 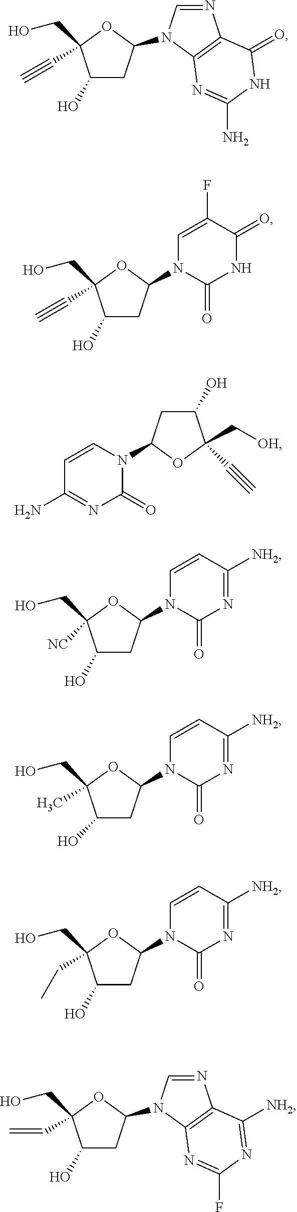 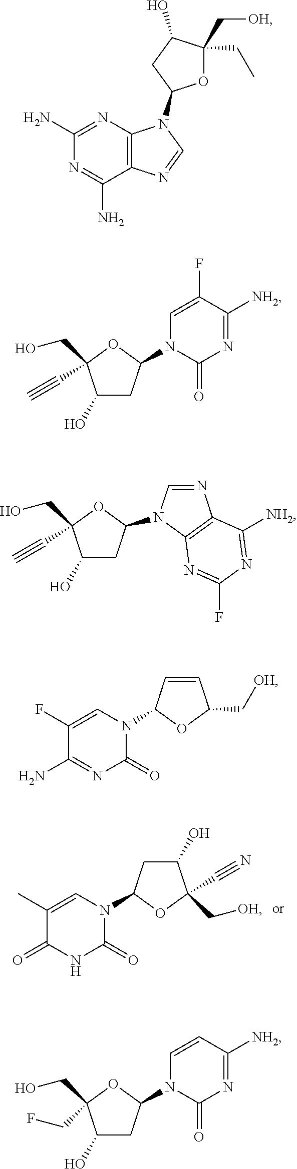
to the patient.

2. The method of claim 1 comprising administering a therapeutically effective amount of: 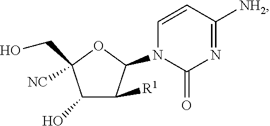
to the patient.

3. The method of claim 1 comprising administering a therapeutically effective amount of: 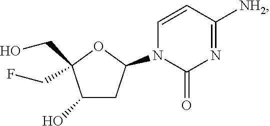 [SMILES: Nc1ccn([C@H]2C[C@H](O)[C@](CO)(CF)O2)c(=O)n1]
to the patient.

4. The method of claim 1, wherein the cancer is breast cancer, colon cancer, lung cancer, pancreatic ductal cancer, prostate cancer, ovarian cancer, or head and neck cancer.

5. The method of claim 4, wherein the cancer is breast cancer.

6. The method of claim 4, wherein the cancer is colon cancer.

7. The method of claim 4, wherein the cancer is lung cancer.

8. The method of claim 4, wherein the cancer is pancreatic ductal cancer.

9. The method of claim 4, wherein the cancer is prostate cancer.

10. The method of claim 4, wherein the cancer is ovarian cancer.

11. The method of claim 4, wherein the cancer is head and neck cancer.

12. The method of claim 1, further comprising administering a therapeutically effective amount of at least one second therapeutic agent useful for treating the cancer.

13. The method of claim 12 for treating breast cancer, wherein the at least one second therapeutic agent is tamoxifen, anastrozole, letrozole, exemestane, trastuzumab, paclitaxel, cyclophosphamide, everolimus, docetaxel, capecitabine, methotrexate, fulvestrant, doxorubicin, pertuzumab, gemcitabine, lapatinib, fluorouracil, palbociclib, abemaciclib, toremifene, eribulin, ado-trastuzumab emtransine, fluoxymesterone, bevacizumab, esterified estrogens, ixabepilone, ribociclib, thiotepa, vinblastine, alpelisib, atezolizumab, fam-trastuzumab deruxtecan, hyaluronidase/trastuzumab, ribociclib/letrozole, talazoparib, sacituzumab or tukatinib.

14. The method of claim 12 for treating colon cancer, wherein the at least one second therapeutic agent is capecitabine, oxaliplatin, fluorouracil, bevacizumab, leucovorin, irinotecan, regorafenib, cetuximab, panitumumab, tipiracil/trifluridine, ziv-aflibercept, interferon beta-1b, methotrexate, pembrolizumab, bevacizumab-awwb, ramucirumab, ipilmumab, nivolumab, encorafenib, levoleucovorin or bevacizumab-bvzr.

15. The method of claim 12 for treating lung cancer, wherein the at least one second therapeutic agent is etoposide, topotecan, nivolumab, pembrolizumab, atezolizumab, durvalumab, methotrexate, cyclophosphamide, carboplatin, cisplatin, docetaxel, gemcitabine, irinotecan, paclitaxel, pemetrexed, vinblastine, or vinorelbine.

16. The method of claim 12 for treating pancreatic ductal cancer, wherein the at least one second therapeutic agent is gemcitabine, fluorouracil, everolimus, erlotinib, paclitaxel, capecitabine, sunitinib, pancreatin, methotrexate, streptozocin, mitomycin, irinotecan, bevacizumab, cetuximab or olaparib.

17. The method of claim 12 for treating head and neck cancer, wherein the at least one second therapeutic agent is cetuximab, docetaxel, methotrexate, pembrolizumab or nivolumab.

18. The method of claim 12 for treating prostate cancer, wherein the at least one second therapeutic agent is buserelin, degarelix, goserelin, histrelin, leuprolide, relugolix, triptorelin, bicalutamide, flutamide, nilutamide, biraterone acetate, apalutamide, or enzalutamide.

19. The method of claim 1, wherein the patient is (a) not infected with the HIV virus, (b) not suspected of being infected with the HIV virus, (c) not being treated for the HIV virus, and/or (d) not being treated to prevent the HIV virus.

20. A kit for carrying out the method of claim 1, the kit comprising (i) the compound; and (ii) and instructions for administering the compound to a patient having cancer.
